SEQUENCE LISTING (9 sequences)

SEQ ID NO: 1 - protein, 10 aa, synthetic construct
RGDSPGERCG

SEQ ID NO: 2 - protein, 11 aa, synthetic construct
ACDCRGDCFCG

SEQ ID NO: 3 - protein, 8 aa, synthetic construct
  MOD_RES 1: note = acetyl modified phenylalanine (modification at N-terminus)
  MOD_RES 8: note = amide-modified glycine (at C-terminus)
FKGGERCG

SEQ ID NO: 4 - protein, 8 aa, synthetic construct
FKGGERCG

SEQ ID NO: 5 - protein, 14 aa, synthetic construct
NQEQVSPLGGERCG

SEQ ID NO: 6 - protein, 6 aa, synthetic construct
GRGDSP

SEQ ID NO: 7 - protein, 5 aa, synthetic construct
  SITE 4: note = D-phenylalanine
  REGION 1..5: note = circular
RGDXV

SEQ ID NO: 8 - protein, 10 aa, synthetic construct
  SITE 4: note = D-phenylalanine
  SITE 9: note = D-phenylalanine
  REGION 1..5: note = circular
  REGION 6..10: note = circular
RGDXKRGDXC

SEQ ID NO: 9 - protein, 12 aa, synthetic construct
KACDCRGDCFCG

(19) United States
(12) Patent Application Publication
(10) Pub. No.: US 2026/0007802 A1
(43) Pub. Date: Jan. 8, 2026

(54) ANNEALED MICROGEL PARTICLE SYSTEMS AND METHODS

(71) Applicant: Tempo Therapeutics, Inc., San Diego, CA (US)

(72) Inventors: Stephanie Deshayes, Santee, CA (US); Westbrook Weaver, San Diego, CA (US)

(21) Appl. No.: 19/279,443

(22) Filed: Jul. 24, 2025

Related U.S. Application Data
(63) Continuation of application No. PCT/US2024/013182, filed on Jan. 26, 2024.
(60) Provisional application No. 63/481,968, filed on Jan. 27, 2023.
(60) Provisional application No. 63/484,439, filed on Feb. 10, 2023.

(51) Int. Cl. A61L 26/00; A61F 2/00

(52) U.S. Cl. CPC A61L 26/008; A61F 2/0059; A61L 26/0019; A61L 26/0023; A61L 26/0095; A61F 2250/0067; A61L 2400/16

(57) ABSTRACT

Provided herein are systems and methods for delivering to the tissue site a dermal filler formulation comprising a hydrogel that anneals in vivo to form a porous covalently stabilized scaffold under conditions sufficient to form a cell matrix within the porous covalently stabilized scaffold effective to permanently fill at least part of a tissue site of a subject with the cell matrix while minimizing a foreign body response in the subject.

### CROSS REFERENCE

This application is a continuation application of International Application No. PCT/US2024/013182, filed Jan. 26, 2024, which claims the benefit of U.S. Provisional Patent Application No. 63/481,968, filed Jan. 27, 2023, and U.S. Provisional Patent Application No. 63/484,439, filed Feb. 10, 2023, each of which is incorporated herein by reference in its entirety for all purposes.

### INCORPORATION BY REFERENCE OF SEQUENCE LISTING

The present application is being filed along with a Sequence Listing in electronic format. The Sequence Listing is provided as a file entitled 48469-707.301_SL.xml, created Jul. 11, 2025, which is 11,871 bytes in size. The information in the electronic format of the Sequence Listing is incorporated by reference in its entirety.

### SUMMARY

In some aspects, the present disclosure provides a method for a method of delivering a dermal filler formulation to a tissue site of a subject, the method comprising: delivering to the tissue site the dermal filler formulation comprising a hydrogel that anneals in vivo to form a porous covalently stabilized scaffold under conditions sufficient to form a cell matrix within the porous covalently stabilized, wherein the cell matrix forms new tissue at the tissue site while minimizing a foreign body response in the subject. In some embodiments, the delivering comprises performing subdermal administration. In some embodiments, the delivering comprises performing dermal administration. In some embodiments, the delivering comprises performing intradermal administration. In some embodiments, the delivering comprises performing subcutaneous administration. In some embodiments, the delivering comprises releasing the dermal filler formulation from a syringe or needle. In some embodiments, the needle has a gauge comprising about 25 gauge to about 35 gauge. In some embodiments, the needle has a gauge comprising about a 27 gauge. In some embodiments, the needle has a gauge comprising about a 30 gauge. In some embodiments, the delivering comprises exerting an extrusion force of up to 40 Newtons (N) on the dermal filler formulation. In some embodiments, the cell matrix comprises cells endogenous to the subject. In some embodiments, at least part of the tissue site is permanently filled by the cell matrix following degradation of the porous covalently stabilized scaffold at the tissue site. In some embodiments, the cell matrix comprises at least 10% of the tissue site following degradation of the porous covalently stabilized scaffold at the tissue site. In some embodiments, the cell matrix comprises at least 25% of the tissue site following degradation of the porous covalently stabilized scaffold at the tissue site. In some embodiments, the cell matrix is formed in less than or equal to about 30 days following the delivering. In some embodiments, the cell matrix begins to form within the scaffold within 7 days after administration. In some embodiments, the cell matrix forms new tissue at the tissue site of the subject before complete degradation of the porous covalently stabilized scaffold. In some embodiments, the new tissue is characterized by having (i) mature vascularization, (ii) a characteristic of surrounding tissue at the tissue site, (iii) or a combination thereof. In some embodiments, the characteristic of the surrounding tissue at the tissue site comprises functionally differentiated cell types from the surrounding tissue. In some embodiments, the new tissue has a persistence time in vivo at the tissue site similar to endogenous tissue surrounding the tissue site. In some embodiments, the tissue site is soft tissue. In some embodiments, the foreign body response is characterized by causing harm to the subject. In some embodiments, the harm is characterized by causing: chronic inflammation, granuloma formation, scar tissue formation, nodule formation, swelling, pain, or any combination thereof. In some embodiments, the harm is caused at the tissue site. In some embodiments, the cell matrix forms while minimizing the foreign body response in the subject when the foreign body response is measured by detecting an amount of granulomas at the tissue site with histological analysis and comparing the amount of granulomas at the tissue site with a reference tissue that does not contain the dermal filler formulation. In some embodiments, the cell matrix forms while minimizing the foreign body response in the subject when the foreign body response is measured by detecting an amount of scar tissue at the tissue site with histological analysis and comparing the amount of scar tissue at the tissue site with a reference tissue that does not contain the dermal filler formulation. In some embodiments, the cell matrix forms while minimizing the foreign body response in the subject when the foreign body response is measured by detecting an amount of nodules at the tissue site with histological analysis and comparing the amount of nodules at the tissue site with a reference tissue that does not contain the dermal filler formulation. In some embodiments, the cell matrix forms while minimizing the foreign body response in the subject when the foreign body response is measured by detecting chronic inflammation at the tissue site with histological analysis. In some embodiments, the cell matrix comprises an amount or a type of collagen mimicking endogenous tissue at the tissue site. In some embodiments, the type of collagen comprises Type I collagen, Type III collagen, or a combination thereof. In some embodiments, Type I collagen is present with Type III collagen in a ratio of less than or equal to about 10:1. In some embodiments, Type I collagen is present with Type III collagen in a ratio of less than or equal to about 6:1. In some embodiments, Type I collagen is present with Type III collagen in a ratio of about 5:1 or less. In some embodiments, at least part of the tissue site comprises elastin following degradation of the porous covalently stabilized scaffold at the tissue site. In some embodiments, the dermal filler formulation is biocompatible with tissue at the tissue site as determined by one or more techniques described by ISO standard 10993. In some embodiments, the porous covalently stabilized scaffold remains at the tissue site in an amount sufficient to fill at least part of the tissue site for an amount of time that is greater than or equal to 9 months following the delivery. In some embodiments, the method further comprises delivering lidocaine to the tissue site. In some embodiments, the lidocaine is delivered at a concentration comprising about 1.0 milligrams per microliter (mg/mL) to about 5.0 mg/mL. In some embodiments, the lidocaine is delivered at a concentration comprising about 3.0 mg/mL. In some embodiments, the hydrogel comprises a polymer comprising hyaluronic acid (HA), poly(ethylene glycol) (PEG), polylactic acid (PLA), collagen, polymethylmethacrylate, or any combination thereof. In some embodiments, the polymer is a co-polymer comprising the HA and the PEG. In some embodiments, the polymer is HA. In some embodiments, the polymer is PEG. In some embodiments, the dermal filler formulation further comprises a vinyl or a derivative thereof. In some embodiments, the vinyl comprises vinyl sulfone (VS), acrylate, methacrylate, acrylamide, maleimide, norbomene, or any combination thereof. In some embodiments, the dermal filler formulation further comprises a thiol or a derivative thereof. In some embodiments, the thiol or the derivative thereof comprises thiolated-HA. In some embodiments, the thiol or the derivative thereof comprises two or more thiols. In some embodiments, the thiol or the derivative thereof comprises a polyethylene glycol (PEG)-dithiol or a derivative thereof. In some embodiments, the hydrogel and the PEG-dithiol or the derivative thereof are delivered to the subject separately. In some embodiments, the hydrogel and the PEG-dithiol or the derivative thereof are delivered to the subject together. In some embodiments, the hydrogel and the PEG-dithiol or the derivative thereof have a shelf life of at least about 18 months when the hydrogel and the PEG-dithiol or the derivative thereof are stored in a single container as a mixture. In some embodiments, the hydrogel and the PEG-dithiol or the derivative thereof have a shelf life of at least about 36 months when the hydrogel and the PEG-dithiol or the derivative thereof are stored in a single container as a mixture at room temperature. In some embodiments, the dermal filler formulation is lyophilized. In some embodiments, the method further comprises reconstituting the dermal filler formulation prior to the delivering the dermal filler formulation to the tissue site. In some embodiments, either of the thiol or the derivative thereof of and the vinyl sulfone or the derivative thereof is present in the dermal filler formulation in excess of the other. In some embodiments, the thiol or the derivative thereof of and the vinyl sulfone or the derivative thereof are present in the dermal filler formulation at a 1:1 molar ratio. In some embodiments, the tissue site comprises: (1) a midface or malar region of the subject, (2) a cheek of the subject, (3) a jawline of the subject, or (4) the lips of the subject, or (5) any combination thereof. In some embodiments, the method further comprises treating the tissue site of the subject by the delivering the dermal filler formulation to the tissue site. In some embodiments, treating the tissue site comprises: tissue filling, dermal filling, removing wrinkles, improving an aesthetic quality of skin surrounding the tissue site, repairing tissue, correcting skin irregularities, treating one or more dermatological conditions, or any combination thereof. In some embodiments, tissue filling comprises: building new tissue formation, generating new tissue formation, or stimulating new tissue formation, or any combination thereof. In some embodiments, the one or more dermatological conditions comprises: acne scars, basal cell carcinoma, cellulitis, epidermolysis bullosa, melanoma, merkel cell carcinoma, scars, skin biopsy, skin cancer, squamous cell carcinoma, stretch marks, or any combination thereof. In some embodiments, treating the tissue site is accomplished by delivering the dermal filler formulation to the tissue site of the subject once. In some embodiments, treating the tissue site is accomplished by delivering the dermal filler formulation to the tissue site of the subject twice. In some embodiments, treating the tissue site is accomplished by delivering the dermal filler formulation to the tissue site of the subject three times. In some embodiments, the porous covalently stabilized scaffold comprises an elastic compressive modulus of about 1,000 Pascals (Pa) to about 100,000 Pa, when the elastic modulus is measured using a compressive test (e.g., on an Instron). In some embodiments, the porous covalently stabilized scaffold comprises a storage modulus of about 50 Pascals (Pa) to about 10,000 Pa, when the storage modulus is measured using a rheometer. In some embodiments, the porous covalently stabilized scaffold comprises a plurality of pores having a median diameter comprising about 5 micrometer (μm) to about 1000 μm. In some embodiments, the dermal filler formulation further comprises a buffer. In some embodiments, the buffer comprises: a phosphate buffer, a 4-(2-hydroxyethyl)-1-piperazineethanesulfonic acid (HEPES) buffer, or an acetate buffer, or any combination thereof.

In some aspects, the present disclosure provides a dermal filler system comprising: (a) microgel particles comprising a hydrogel polymer and a thiol or a derivative thereof, wherein the hydrogel polymer comprises hyaluronic acid (HA), poly(ethylene glycol) (PEG), polylactic acid (PLA), or a combination thereof; and (b) vinyl sulfone (VS) or a derivative thereof, wherein the microgel particles undergo an annealing reaction to form a porous covalently stabilized scaffold, wherein either of the thiol or the derivative thereof of and the vinyl sulfone or the derivative thereof is present in the dermal filler system in excess of the other. In some aspects, the present disclosure provides a dermal filler system comprising: (a) a dermal filler formulation comprising microgel particles, wherein the microgel particles comprise a hydrogel polymer and a thiol or a derivative thereof, wherein the hydrogel polymer comprises hyaluronic acid (HA), poly(ethylene glycol) (PEG), polylactic acid (PLA), or a combination thereof; and (b) vinyl sulfone (VS) or a derivative thereof, wherein the microgel particles undergo an annealing reaction to form a porous covalently stabilized scaffold comprising an elastic modulus of about 1,000 Pascals (Pa) to about 100,000 Pa. In some embodiments, the microgel particles are spherical. In some embodiments, the microgel particles comprise microspheres. In some embodiments, the microgel particles comprise diameters comprising 5 μm to 1000 μm. In some embodiments, the diameters comprise between 50 μm to 1000 μm. In some embodiments, the diameters comprise between 80 μm to 140 μm. In some embodiments, the porous covalently stabilized scaffold comprises pores comprise a median pore diameter of about 5 μm and above. In some embodiments, the pores comprise a median pore diameter of about 10 μm to about 35 μm. In some embodiments, the microgel particles further comprise one or more cell adhesive peptides. In some embodiments, the one or more cell adhesive peptides comprises an RGD peptide. In some embodiments, the RGD peptide comprises an amino acid sequence provided in any one of SEQ ID NOS: 1-2 or 6-9. In some embodiments, the RGD peptide comprises an amino acid sequence that is about 75% identical to an amino acid sequence provided in any one of SEQ ID NOS: 1-3. In some embodiments, the microgel particles further comprise one or more K peptides. In some embodiments, the one or more K peptides comprises an amino acid sequence provided in any one of Ac-FKGGERCG-NH2 (SEQ ID NO: 3). In some embodiments, the microgel particles further comprise one or more Q peptides. In some embodiments, the one or more Q peptides comprises an amino acid sequence provided in any one of SEQ ID NO: 4. In some embodiments, the hydrogel polymer comprises a polydispersity of no more than 0.1. In some embodiments, the polydispersity is calculated based on a standard deviation and mean size of the particles (e.g., PDI=(SD/mean)\hat{ }2). In some embodiments, the dermal filler further comprises a buffer, wherein the buffer comprises: a phosphate buffer, a 4-(2-hydroxyethyl)-1-piperazineethanesulfonic acid (HEPES) buffer, or an acetate buffer, or any combination thereof. In some embodiments, the dermal filler system further comprises lidocaine. In some embodiments, the lidocaine is present in the dermal filler system at a concentration of about 1.0 mg/mL to about 5.0 mg/mL. In some embodiments, the lidocaine is present in the dermal filler system at a concentration of about 3.0 mg/mL. In some embodiments, the hydrogel polymer comprises the HA and the PEG. In some embodiments, the hydrogel is a copolymer of the HA and the PEG having approximately identical molecular weights of each of the HA and the PEG. In some embodiments, the HA comprises a molecular weight of 1 kilodalton (kDa) to 1 megadalton (1 MDa). In some embodiments, the HA comprises a molecular weight of 10 kDa to 250 kDa (e.g., 10, 40, 50, 150, and 250 kDa). In some embodiments, the PEG comprises a molecular weight of 1 kilodalton (kDa) to 5 kDa. In some embodiments, the hydrogel polymer comprises the thiol or the derivative thereof or the VS or the derivative thereof, or the combination thereof. In some embodiments, the HA is modified to comprise the thiol or the derivative thereof to form thiolated-HA. In some embodiments, the PEG is modified to comprise the VS or the derivative thereof to form PEG-VS. In some embodiments, the PEG-VS comprises a multi-arm PEG-VS. In some embodiments, the multi-arm PEG-VS comprises, 4-arm or 8-arm PEG-VS. In some embodiments, the VS comprises divinyl sulfone. In some embodiments, the thiol or the derivative thereof and the VS or the derivative thereof are configured to interact with each other in a reaction to synthesize the microgel particles. In some embodiments, the reaction comprises a covalent synthesizing reaction. In some embodiments, the covalent synthesizing reaction comprises a Michael addition (e.g., thiol-ene Michael addition) or a pseudo-Michael addition reaction. In some embodiments, the thiol or the derivative thereof is a Michael donor in the Michael addition or pseudo-Michael addition reaction. In some embodiments, the VS or derivative thereof is a Michael acceptor in the Michael addition or pseudo-Michael addition reaction. In some embodiments, the thiol or the derivative thereof and the VS or the derivative thereof are present in the dermal filler system at a molar ratio of about 1:1. In some embodiments, there is an excess of either of the thiol or the derivative thereof and the VS or the derivative thereof in the dermal filler system such that the excess of either of the thiol or the derivative thereof or the VS or the derivative thereof participates in the annealing reaction to form the porous covalently stabilized scaffold. In some embodiments, the dermal filler system further comprises two or more acrylates, methacrylates, acrylamides, maleimides, norbornenes, or any combination thereof. In some embodiments, the dermal filler system further comprises a molecule comprising two or more thiols or derivatives thereof. In some embodiments, the molecule comprises PEG. In some embodiments, the molecule comprises PEG-dithiol. In some embodiments, the PEG-dithiol comprises a molecular weight of about 1.0 kDa to about 5.0 kDa. In some embodiments, the PEG-dithiol comprises a molecular weight of about 3.4 kDa. In some embodiments, the PEG-dithiol comprises linear PEG-dithiol, multi-arm PEG-dithiol, or a combination thereof. In some embodiments, the multi-arm PEG-dithiol comprises 4-arm or 8-arm PEG-dithiol. In some embodiments, the PEG-dithiol is configured to interact with the excess VS or derivative thereof in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the thiol or derivative thereof of the PEG-dithiol is a Michael donor in the Michael addition or pseudo-Michael addition reaction. In some embodiments, the excess VS is a Michael acceptor in the Michael addition or pseudo-Michael addition reaction. In some embodiments, the dermal filler system further comprises PEG-divinyls sulfone or a derivative thereof. In some embodiments, the PEG-divinyl sulfone or derivative thereof is configured to interact with the excess thiol or derivative thereof in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the divinyl sulfone or derivative thereof of the PEG-divinyl sulfone is a Michael acceptor in the Michael addition or pseudo-Michael addition reaction. In some embodiments, the excess thiol is a Michael donor in the Michael addition or pseudo-Michael addition reaction. In some embodiments, the hydrogel polymer comprises the HA. In some embodiments, the HA comprises a molecular weight of 1 kilodalton (kDa) to 1000 kDa. In some embodiments, the HA comprises a molecular weight of about 10 kDa to about 250 kDa. In some embodiments, the molecular weight comprises about 10, 40, 50, 150, or 250 kDa. In some embodiments, the dermal filler system further comprises glutaraldehyde or a derivative thereof, divinyl sulfone or a derivative thereof, 1,4-butanediol diglycidyl ether (BDDE) or a derivative thereof, or any combination thereof configured to interact in a crosslinking reaction to synthesize the microgel particles. In some embodiments, the HA is modified to comprise the thiol or the derivative thereof to form thiolated-HA. In some embodiments, the HA is modified to comprise the VS or the derivative thereof to form HA-VS. In some embodiments, the thiol or the derivative thereof and the VS or derivative thereof are configured to interact in a crosslinking reaction to synthesize the microgel particles. In some embodiments, the crosslinking reaction comprises a covalent synthesizing reaction. In some embodiments, the covalent synthesizing reaction comprises a Michael addition or pseudo-Michael addition reaction. In some embodiments, the thiol or the derivative thereof is a Michael donor in the Michael addition or pseudo-Michael addition reaction. In some embodiments, the VS or the derivative thereof is a Michael acceptor in the Michael addition or pseudo-Michael addition reaction. In some embodiments, the thiol or the derivative thereof and the VS or derivative thereof are present in the dermal filler system at a molar ratio of about 1:1. In some embodiments, the thiol or the derivative thereof and the VS or the derivative thereof are in excess of each other in the dermal filler system such that the excess of the thiol or the derivative thereof or the VS or the derivative thereof participates in the annealing reaction to form the porous covalently stabilized scaffold. In some embodiments, the dermal filler system further comprises two or more acrylates, methacrylates, acrylamides, maleimides, norbomenes, or any combination thereof. In some embodiments, the dermal filler system further comprises a molecule comprising two or more thiols or derivatives thereof. In some embodiments, the molecule comprises PEG. In some embodiments, the molecule comprises PEG-dithiol. In some embodiments, the PEG-dithiol comprises a molecular weight of about 1.0 kDa to about 5.0 kDa. In some embodiments, the PEG-dithiol comprises a molecular weight of about 3.4 kDa. In some embodiments, the PEG-dithiol comprises linear PEG-dithiol, multi-arm PEG-dithiol, or a combination thereof. In some embodiments, the multi-arm PEG-dithiol comprises 4-arm or 8-arm PEG-dithiol. In some embodiments, the PEG-dithiol is configured to interact with the excess VS or derivative thereof in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the thiol or derivative thereof of the PEG-dithiol is a Michael donor in the Michael addition or pseudo-Michael addition reaction. In some embodiments, the excess VS is a Michael acceptor in the Michael addition or pseudo-Michael addition reaction. In some embodiments, the dermal filler system further comprises PEG-divinyls sulfone or a derivative thereof. In some embodiments, the PEG-divinyl sulfone or derivative thereof is configured to interact with the excess thiol or derivative thereof in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the divinyl sulfone or derivative thereof of the PEG-divinyl sulfone is a Michael acceptor in the Michael addition or pseudo-Michael addition reaction. In some embodiments, the excess thiol is a Michael donor in the Michael addition or pseudo-Michael addition reaction. In some embodiments, he hydrogel polymer comprises the PEG. In some embodiments, the PEG comprises a molecular weight of 1 kilodalton (kDa) to 1000 kDa. In some embodiments, the hydrogel polymer further comprises the thiol or the derivative thereof, the VS or the derivative thereof, or a combination thereof. In some embodiments, the PEG comprises the thiol or the derivative thereof to form PEG-dithiol. In some embodiments, the PEG comprises the VS or the derivative thereof to form PEG-VS. In some embodiments, the PEG-VS groups comprises multi-arm PEG-VS. In some embodiments, the multi-arm PEG-VS comprises, 4-arm or 8-arm PEG-VS. In some embodiments, the VS comprises divinyl sulfone. In some embodiments, the thiol or the derivative thereof and the VS or the derivative thereof are configured to interact with each other in a reaction to synthesize the microgel particles. In some embodiments, the reaction comprises a covalent synthesizing reaction. In some embodiments, the covalent synthesizing reaction comprises a Michael addition (e.g., thiol-ene Michael addition) or a pseudo-Michael addition reaction. In some embodiments, the thiol or the derivative thereof is a Michael donor in the Michael addition or pseudo-Michael addition reaction. In some embodiments, the VS or derivative thereof is a Michael acceptor in the Michael addition or pseudo-Michael addition reaction. In some embodiments, the thiol or the derivative thereof and the VS or the derivative thereof are present in the dermal filler system at a molar ratio of about 1:1. In some embodiments, there is an excess of either of the thiol or the derivative thereof and the VS or the derivative thereof in the dermal filler system such that the excess of either of the thiol or the derivative thereof or the VS or the derivative thereof participates in the annealing reaction to form the porous covalently stabilized scaffold. In some embodiments, the dermal filler system further comprises two or more acrylates, methacrylates, acrylamides, maleimides, norbomenes, or any combination thereof. In some embodiments, the dermal filler system further comprises a molecule comprising two or more thiols or derivatives thereof. In some embodiments, the molecule comprises PEG. In some embodiments, the molecule comprises PEG-dithiol. In some embodiments, the PEG-dithiol comprises a molecular weight of about 1.0 kDa to about 5.0 kDa. In some embodiments, the PEG-dithiol comprises a molecular weight of about 3.4 kDa. In some embodiments, the PEG-dithiol comprises linear PEG-dithiol, multi-arm PEG-dithiol, or a combination thereof. In some embodiments, the multi-arm PEG-dithiol comprises 4-arm or 8-arm PEG-dithiol. In some embodiments, the PEG-dithiol is configured to interact with the excess VS or derivative thereof in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the thiol or derivative thereof of the PEG-dithiol is a Michael donor in the Michael addition or pseudo-Michael addition reaction. In some embodiments, the excess VS is a Michael acceptor in the Michael addition or pseudo-Michael addition reaction. In some embodiments, the dermal filler system further comprises PEG-divinyls sulfone or a derivative thereof. In some embodiments, the PEG-divinyl sulfone or derivative thereof is configured to interact with the excess thiol or derivative thereof in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the divinyl sulfone or derivative thereof of the PEG-divinyl sulfone is a Michael acceptor in the Michael addition or pseudo-Michael addition reaction. In some embodiments, the excess thiol is a Michael donor in the Michael addition or pseudo-Michael addition reaction. In some embodiments, the porous covalently stabilized scaffold is degradable in vivo by one or more degradation pathways. In some embodiments, the one or more degradation pathways comprises oxidative degradation, enzymatic degradation, photodegradation, or hydrolytic degradation. In some embodiments, the porous covalently stabilized scaffold is present in the tissue site for at least 18 months before complete degradation. In some embodiments, the porous covalently stabilized scaffold is present in the tissue site for at least 24 months before complete degradation. In some embodiments, the microgel particles are present in a suspension comprising the microgel particles and water. In some embodiments, a 50% to 100% volume fraction of the suspension comprises the microgel particles. In some embodiments, the volume fraction of the microgel particles is greater than or equal to about 50% when the dermal filler is formulated for administration with a needle. In some embodiments, the porous covalently stabilized scaffold comprises an elastic compressive modulus of 1,000 Pascals (Pa) to 50,000 Pa. In some embodiments, the porous covalently stabilized scaffold comprises an elastic compressive modulus of 5,000 Pascals (Pa) to 100,000 Pa in an unswollen state. In some embodiments, the porous covalently stabilized scaffold comprises an elastic compressive modulus of 1,000 Pascals (Pa) to 50,000 Pa in a swollen state. In some embodiments, the porous covalently stabilized scaffold comprises a storage modulus of 50 Pascals (Pa) to 10,000 Pa. In some embodiments, the porous covalently stabilized scaffold comprises a storage modulus of 60 Pa to 1,000 Pa. In some embodiments, the porous covalently stabilized scaffold comprises a loss modulus of about 10 Pascals (Pa) to 10,000 Pa. In some embodiments, the porous covalently stabilized scaffold comprises an elastic compressive modulus of greater than or equal to about 50 Pa when the dermal filler is formulated for administration with a needle. In some embodiments, the thiol or the derivative thereof and the vinyl sulfone or the derivative thereof are present in the dermal filler system at a molar ratio of about 0.3 to about 0.8 to achieve the elastic compressive modulus. In some embodiments, the microgel particles are present in a suspension comprising the microgel particles and water and wherein a 50% to 100% volume fraction of the suspension comprises the microgel particles to achieve the elastic compressive modulus. In some embodiments, the covalently stabilized scaffold comprises an apparent viscosity of 1000 to about 1000000 mPa*s. In some embodiments, the porous covalently stabilized scaffold comprises a pH of 5.0 to 9.0. In some embodiments, the porous covalently stabilized scaffold comprises a pH of 6.5 to 7.5. In some embodiments, the porous covalently stabilized scaffold comprises an osmolality of about 100 milliosmole per kilogram (mOsmol/kg) to about 400 mOsmol/kg. In some embodiments, the hydrogel polymer comprises a degree of substitution per monomer of about 5% to about 20%. In some embodiments, the hydrogel polymer comprises modified HA. In some embodiments, the system is lyophilized. In some embodiments, the microgel particles comprise an elastic modulus of about 10 kPa to about 100 kPa. In some embodiments, the microgel particles comprise an elastic modulus of about 15 kPa to about 50 kPa.

In some aspects, the present disclosure provides an aesthetic formulation, comprising: the dermal filler system described herein in a suspension, wherein the suspension comprises a buffer and a molecule comprising two or more thiols or derivatives thereof, two or more vinyls or derivatives thereof, or a combination thereof. In some embodiments, the buffer comprises: a phosphate buffer, a 4-(2-hydroxyethyl)-1-piperazineethanesulfonic acid (HEPES) buffer, or an acetate buffer, or any combination thereof. In some embodiments, the molecule comprises PEG-dithiol. In some embodiments, the aesthetic formulation is formulated for administration to a subject. In some embodiments, the administration is subdermal administration, dermal administration, intradermal administration, or subcutaneous administration. In some embodiments, administration minimizes a foreign body response in the subject. In some embodiments, the aesthetic formulation comprises a dose volume of about 0.75 mL to about 1.0 mL. In some embodiments, the aesthetic formulation is sterile. In some embodiments, the aesthetic formulation further comprises lidocaine.

In some aspects, the present disclosure provides a delivery device, comprising: (a) a body comprising the dermal filler system described herein or the aesthetic formulation described herein; and (b) an applicator in fluidic communication with the body, wherein the delivery device is sterile. In some embodiments, the delivery device is a syringe or needle. In some embodiments, the delivery device is a microneedle patch.

In some aspects, the present disclosure provides a method of lyophilizing the dermal filler system described herein or the aesthetic formulation described herein comprising lyophilizing the dermal filler system or aesthetic formulation into a powder. In some embodiments, the method further comprises reconstituting the lyophilized dermal filler system or aesthetic formulation for delivery to a subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 illustrates a work flow for starting and conducting rheology measurements including flow curves for apparent viscosity measurement and frequency or amplitude sweeps for storage and loss modulus measurements, according to some embodiments herein.

FIGS. 2A-2B shows viscosity curves illustrating the power law region, according to some embodiments herein. FIG. 2A shows a complete viscosity curve, according to some embodiments herein. FIG. 2B shows a viscosity curve for the power law region only, according to some embodiments herein. As shown in FIGS. 2A-2B, higher particle stiffnesses (15 kPa, 30 kPa, and 46 kPa) are generally associated with higher viscosities, according to some embodiments herein.

FIGS. 3A-3C shows viscosity curves at constant volume fractions (VF) and different microgel particle stiffnesses, according to some embodiments herein. FIG. 3A shows viscosity curves for varying unswollen microgel particle stiffnesses at a microgel particle volume fraction (VF) of 0.75, according to some embodiments herein. FIG. 3B shows viscosity curves for varying microgel particle stiffnesses at a microgel particle volume fraction (VF) of 0.85, according to some embodiments herein. FIG. 3C shows viscosity curves for varying microgel particle stiffnesses at a microgel particle volume fraction (VF) of 0.95, according to some embodiments herein. As shown in FIGS. 3A-3C, higher particle stiffnesses (15 kPa, 20 kPa, 30 kPa, and 46 kPa) are associated with higher viscosities, according to some embodiments herein.

FIGS. 4A-4D shows viscosity curves at constant microgel particle stiffnesses and different volume fractions, according to some embodiments herein. FIG. 4A shows viscosity curves for varying microgel particle volume fractions at an unswollen microgel particle stiffness of 15 kilopascals (kPa), according to some embodiments herein. FIG. 4B shows viscosity curves for varying microgel particle volume fractions at an unswollen microgel particle stiffness of 20 kPa, according to some embodiments herein. FIG. 4C shows viscosity curves for varying microgel particle volume fractions at an unswollen microgel particle stiffness of 30 kPa, according to some embodiments herein. FIG. 4D shows viscosity curves for varying microgel particle volume fractions at an unswollen microgel particle stiffness of 45 kPa, according to some embodiments herein. As shown in FIGS. 4A-4D, higher volume fractions (0.75 mL/mL, 0.85 mL/mL, and 0.95 mL/mL) are associated with higher viscosities, according to some embodiments herein.

FIG. 5 illustrates the effects of volume fraction (VF) and microgel particle stiffness on the elastic modulus (EM) of the annealed scaffold, according to some embodiments herein. As shown in FIG. 5, higher particle stiffnesses (15 kPa, 20 kPa, 30 kPa, and 46 kPa) are associated with higher elastic moduli of the annealed scaffold, according to some embodiments herein.

FIG. 6A illustrates the dependence of viscosity on microgel particle volume fractions (VF) at different unswollen microgel particle stiffnesses measured at a shear rate of 0.1 inverse seconds (s⁻¹), according to some embodiments herein. FIG. 6B illustrates the dependence of viscosity on unswollen microgel particle stiffnesses at different volume fractions (VF) measured at a shear rate of 0.1 s⁻¹, according to some embodiments herein. FIG. 6C illustrates the dependence of viscosity on microgel particle volume fractions (VF) at different unswollen microgel particle stiffnesses measured at a shear rate of 1.0 s⁻¹, according to some embodiments herein. FIG. 6D illustrates the dependence of viscosity on unswollen microgel particle stiffnesses at different volume fractions (VF) measured at a shear rate of 1.0 s⁻¹, according to some embodiments herein. FIG. 6E illustrates the dependence of viscosity on microgel particle volume fractions (VF) at different unswollen microgel particle stiffnesses measured at a shear rate of 10.0 s⁻¹, according to some embodiments herein. FIG. 6F illustrates the dependence of viscosity on unswollen microgel particle stiffnesses at different volume fractions (VF) measured at a shear rate of 10.0 s⁻¹, according to some embodiments herein. As shown in FIGS. 6A, 6C, and 6E, higher particle stiffnesses (15 kPa, 20 kPa, 30 kPa, and 46 kPa) are associated with higher viscosities, according to some embodiments herein. As shown in FIGS. 6B, 6D, and 6F, higher volume fractions (0.75 mL/mL, 0.85 mL/mL, and 0.95 mL/mL) are associated with higher viscosities, according to some embodiments herein.

FIG. 7 illustrates rheologic operating ranges of microgel particle suspensions from viscosity curves, according to some embodiments herein.

FIG. 8A shows anatomical injection site locations, according to some embodiments herein. FIG. 8B shows study design parameters, according to some embodiments herein. FIG. 8C shows a schematic of injection site anatomy, according to some embodiments herein. FIG. 8D shows an image of an excised tissue injection site, according to some embodiments herein.

FIG. 9 illustrates microgel particle synthesis through Michael addition, according to some embodiments herein.

FIG. 10 illustrates cell viability in vitro when exposing cells in culture to microgel particle systems described herein. As shown, each grouping of bar graphs depicts, from left to right, day 1, day 3, and day 6, respectively.

FIG. 11 illustrates cell viability in the presence of PETMA at different concentrations, according to some embodiments herein. As shown, each grouping of bar graphs depicts, from left to right, day 1, day 3, and day 6, respectively.

FIG. 12 illustrates cell viability in the presence of various annealing reactions, according to some embodiments herein. As shown, each grouping of bar graphs depicts, from left to right, day 1, day 3, and day 6, respectively.

FIG. 13 illustrates an example injection site of dermal filler systems disclosed herein in a rat, according to some embodiments herein.

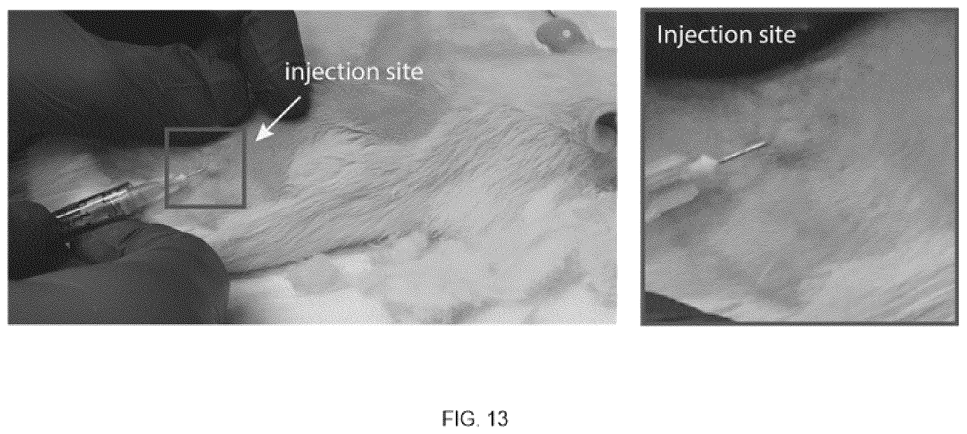

FIG. 14A shows histology of formulation 4 in a rat at 7 days post injection and at four insets, according to some embodiments herein. FIG. 14B shows histology of formulation 4 in a rat at 7 days post injection and at four insets, according to some embodiments herein.

FIG. 15A shows histology of formulation 4 in a rat at 30 days post injection and at four insets, according to some embodiments herein. FIG. 15B shows histology of formulation 4 in a rat at 30 days post injection and at four insets, according to some embodiments herein.

FIG. 16A shows degradation, immune response, new protein deposition, and cellular infiltration for formulation 2, according to some embodiments herein. FIG. 16B shows degradation, immune response, new protein deposition, and cellular infiltration for formulation 3, according to some embodiments herein. FIG. 16C shows degradation, immune response, new protein deposition, and cellular infiltration for formulation 4, according to some embodiments herein. FIG. 16D shows degradation, immune response, new protein deposition, and cellular infiltration for Juvederm, according to some embodiments herein.

FIG. 17 illustrates histology of injection sites after administration of dermal filler systems described herein, demonstrating vascular ingrowth (red arrows), protein deposition within pores (blue arrows), and collagen bundles (green arrows), according to some embodiments herein.

FIG. 18 illustrates a scheme for thiolation of Hyaluronic Acid (HA) prior to synthesis of microgel particles, according to some embodiments herein.

FIGS. 19A-19B illustrates the elastic modulus (EM) of swollen and unswollen gels with varying ratios of PEG-VS to SH-HA, according to some embodiments herein. FIG. 19A shows the dependence of EM of swollen and unswollen gels on the concentration of SH-HA (at a fixed concentration of PEG-VS) when the hyaluronic Acid (HA) comprises a molecular weight of 10 kDa, according to some embodiments herein. FIG. 19B shows the dependence of EM of swollen and unswollen gels on the concentration of SH-HA (at a fixed concentration of PEG-VS) when the Hyaluronic Acid (HA) comprises a molecular weight of 50 kDa, according to some embodiments herein.

FIG. 20 illustrates the linear dependence of thiol concentration to SH-HA concentration when the hyaluronic acid (HA) comprises either a molecular weight of 10 kDa or 50 kDa, according to some embodiments herein.

FIG. 21 illustrates the effects of different annealing agents on the elastic modulus (EM) of annealed scaffolds, according to some embodiments herein.

FIG. 22 illustrates the effect of formulation pH on the kinetics of the annealing reaction when the annealing agent comprises a linear PEG-(SH)₂ with molecular weight of 3.4 kDa and a volume fraction of 80%, according to some embodiments herein.

FIG. 23 illustrates the annealing kinetics, such as elastic modulus, of annealed microgel particles for a dermal filler system using PEG-dithiol, according to some embodiments herein.

FIGS. 24A-24C illustrate the effect of lidocaine on elastic modulus of the annealed microgel particles, according to some embodiments herein. FIG. 24A shows the effect of lidocaine on elastic modulus of a formulation, according to some embodiments herein. FIG. 24B shows the effect of lidocaine on elastic modulus of a formulation, according to some embodiments herein. FIG. 24C shows the effect of lidocaine on elastic modulus of a formulation, according to some embodiments herein.

FIGS. 25A-25B illustrate the proliferation of mouse fibroblast cell line 3T3 cells after treatment with varying concentrations of annealing agents, according to some embodiments herein. FIG. 25A shows the proliferation of 3T3 cells after treatment with varying concentrations of an annealing agent comprising 4-ARM-PEG-SH at a molecular weight of 20 kDa, according to some embodiments herein. FIG. 25B shows the proliferation of 3T3 cells after treatment with varying concentrations of an annealing agent comprising linear PEG-(SH)₂ at a molecular weight of 3.4 kDa, according to some embodiments herein. As shown, each grouping of bar graphs depicts, from left to right, day 1, day 3, and day 6, respectively.

FIG. 26 illustrates the survival of 3T3 cells 6 days after exposure to either 4-ARM-PEG-SH at a molecular weight of 20 kDa or linear PEG-(SH)₂ at a molecular weight of 3.4 kDa, according to some embodiments herein.

FIG. 27 illustrates the proliferation of 3T3 cells after exposure to either 4-ARM-PEG-SH with free thiols or 4-ARM-PEG-SH that has been fully capped by maleimide to remove the free thiols at a molecular weight of 20 kDa, according to some embodiments herein. The proliferation of 3T3 cells after exposure to maleimide alone is provided as a control. As shown, each grouping of bar graphs depicts, from left to right, day 1 and day 3, respectively.

FIG. 28 illustrates the proliferation of 3T3 cells after treatments with varying dermal filler systems and annealing reactions, according to some embodiments herein. Note that data presented in FIG. 29 and FIG. 30 come from separately performed experiments. As shown, each grouping of bar graphs depicts, from left to right, day 1, day 3, and day 6, respectively.

FIG. 29 illustrates the proliferation of 3T3 cells after treatments with varying dermal filler systems and annealing reactions, according to some embodiments herein. Note that data presented in FIG. 29 and FIG. 30 come from separately performed experiments. As shown, each grouping of bar graphs depicts, from left to right, day 1, day 3, and day 6, respectively.

FIG. 30 illustrates the proliferation of 3T3 cells after treatments with varying dermal filler systems and annealing reactions, and for a number of experiments, normalized to the control experiments for cell proliferation without exposure to any dermal filler system, according to some embodiments herein.

FIG. 31 illustrates the degradation of HA particles over time using hyaluronidase, according to some embodiments herein.

FIGS. 32A-32B illustrate break force and extrusion before and after annealing, according to some embodiments herein. FIG. 32A illustrates the break force and extrusion force for dermal filler systems after annealing, according to some embodiments herein. FIG. 32B illustrates the break force and extrusion force for dermal filler systems before annealing, according to some embodiments herein. A 30G needle was used with a plunger speed of 10 mm/min for this experiment.

FIG. 33 illustrates extrusion force plots over time for dermal filler systems, according to some embodiments herein.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

The term “about,” as used herein, with reference to a number refers to that number plus or minus 10% of that number. The term “about” a range refers to that range minus 10% of its lowest value and plus 10% of its greatest value.

The term, “annealing agent,” as used herein, refers to an entity capable of inducing the annealing reaction between particles of the present disclosure (e.g., microparticles) to form an annealed scaffold (e.g., covalently stabilized scaffold). Non-limiting examples of annealing agents include Eosin Y, PETMA (Pentaerythritol tetrakis(2-mercaptoacetate))], Factor XIII/FactorXIIIa, molecules with two or more reactive functional groups including thiols (e.g., PEG-dithiol), divinyl sulfone, or a combination thereof. An annealing agent may not covalently participate in the linkage of the particles described herein when the annealing reaction is induced. An annealing agent may be covalently linked to the particles of the annealed scaffold when the annealing reaction is induced.

The term, “annealing component,” as used herein, refers to a substrate in an annealing reaction between microgel particles of the present disclosure (e.g., microparticles) that is bound to the microgel particles themselves. Non-limiting examples of annealing components include K or Q peptides, two or more reactive functional groups including thiol or thiol derivatives, vinyl or vinyl derivatives (e.g., vinyl sulfone), methacrylates, acrylates, amines, or a combination thereof.

The term “biocompatible,” as used herein, refers to biocompatibility as determined under the International Standard ISO 10993-1, which is hereby incorporated by reference in its entirety.

The term, “cell adhesive peptide,” or “cell adhesion peptide,” as used herein interchangeably refers to peptides capable of initiating cell adhesion to a synthetic material, such as a microgel particle. A non-limiting example of cell adhesive peptides is an RGD peptide. The cell adhesion peptide disclosed herein may be provided in Moral MEG, Siahaan T J. Conjugates of Cell Adhesion Peptides for Therapeutics and Diagnostics Against Cancer and Autoimmune Diseases. Curr Top Med Chem. 2017; 17(32):3425-3443, which is hereby incorporated by reference in its entirety.

The term “cell matrix” as used herein refers to a network of proteins or other molecules that surround, support, and/or give structure to cells and tissues in the body.

The term, “elastic compressive modulus,” as used herein, refers to the stiffness of either individual microgel particles, macroscopic hydrogels, or annealed scaffolds of microgel particles. Elastic compressive modulus may be measured by compressive testing (failure or non-failure) in which an anvil of known cross-sectional area is depressed into a hydrogel, non-annealed scaffold (microgel particles), or annealed scaffold at a known distance and speed, while a force transducer attached to the anvil records the force placed on the anvil. The elastic compressive modulus may be mathematically calculated from the stress/strain curves recorded during compression testing.

The term, “crosslinker,” as used herein, refers to a reagent that participates in the crosslinking reaction of raw materials to form a microgel particle of the present disclosure (e.g., microparticles). A crosslinkers is a linker with two or more reactive functional groups (e.g., thiol, vinyl sulfone, maleimide, acrylate, methacrylate, acrylamide, methacrylamide, norbornene, amine, hydroxyl). When a crosslinker is in excess in the crosslinker reaction, a crosslinker may also be an annealing component and participate with the annealing agent in an annealing reaction between particles of the present disclosure. Non-limiting examples of crosslinkers include vinyl derivatives with two or more vinyl groups (e.g., PEG-VS), thiol derivatives with two or more thiol groups (e.g., PEG-dithiol or thiolated HA), peptides with two or more cysteines (e.g., matrix metalloproteinase (MMP)-degradable crosslinker), or the combination thereof.

The term, “crosslinking,” as used herein, refers to a reaction to form the microgel particle of the present disclosure (e.g., microparticles).

The term “derivative” in reference to a “vinyl” or a “thiol” refers to a vinyl-containing chemical entity or a thiol-containing chemical entity, respectively. Non-limiting examples of vinyl derivatives include PEG-VS, PEG-acrylate, PEG-methacrylate, PEG-maleimide. Non-limiting examples of vinyl groups include vinyl sulfone, acrylate, methacrylate, acrylamide, maleimide, and norbornene. Non-limiting thiol derivatives include PEG-dithiol, thiolated HA, cysteine-containing peptides (e.g., matrix metalloproteinase (MMP)-degradable crosslinker), any organosulfur compound of the form R—SH, where R represents an alkyl, or other organic substituent, methanethiol, ethanethiol, 1-propanethiol, 2-propoanethiol, allyl mercaptan, butanethiol, tert-butyl mercaptan, pentanethiols, thiophenol, dimercaptosuccinic acid, thioacetic acid, coenzyme A, glutathione, metallothionein, cysteine, 2-mercaptoethanol, dithiothreitol, dithioerythritol, 1-mercaptoindole, grapefruit mercaptan, furan-2-ylmethanethiol, 3-mercaptopropane-1, 2-diol, 3-mercapto-1-propanesulfonic acid, 1-hexadecanethiol, pentachlorobenzenethiol, or a combination thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term, “elastic modulus,” as used herein, refers to a mechanical property of a substance related to resistance of being deformed elastically when stress is applied to it, which may be calculated with the following equation:

$$δ {=}^{def} \frac{stress}{strain} ,$$

where stress is the force causing the deformation divided by the area to which the force is applied and strain is the ratio of the change in some parameter caused by the deformation to the original value of the parameter. Since strain is a dimensionless quantity, the units of δ, will be the same as the units of stress. Elastic modulus may be measured by mechanical testing (failure or non-failure) in which a force transducer is attached to a specimen in a manner that creates a mechanical continuum between the transducer and the specimen. The specimen may then be deformed either by compressing it, stretching it, or shearing it, and the anvil records the force placed on the anvil by the specimen as it deforms. The elastic modulus may be mathematically calculated from the stress/strain curves recorded during mechanical testing. Different types of elastic moduli may be measured based on the type of deformation of the specimen. In compressive deformation, the compressive modulus may be calculated. In stretching, the tensile modulus is calculated. In shear deformation, the shear modulus may be calculated.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay or on a piece of tissue that has been excised (removed) from a subject.

The term “foreign body response,” as used herein, refers to a fibrotic response typically resulting from an implant or dermal filler that is characterized by chronic inflammation, granuloma formation, and/or scar tissue formation, at or around the site of implantation. A foreign body response can be detected in a subject by histological analysis of the tissue at or around the site of implantation, and comparing the results of the histological analysis with histology of a reference tissue that does not contain the implant or dermal filler.

The term “gel,” as used herein, refers to three-dimensional network of crosslinked polymers swollen in a solvent.

The term “HA,” as used herein, refers to hyaluronic acid or hyaluronan.

The term “HEPES,” as used herein, refers to 4-(2-hydroxyethyl)-1-piperazineethanesulfonic acid.

The term “in situ,” as used herein, refers to the original site of delivery or administration, confined to the site of original site without the invasion of neighboring tissues.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “in vivo” is used to describe an event that takes place in a subject's body.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

The term “hydrogel,” as used herein, refers to a gel that is water-insoluble and capable of holding water.

The term, “K peptide,” as used herein, refers to a peptide comprising an amino acid sequence comprising one or more lysine residues that serve as a substrate for an annealing agent in an annealing reaction.

The term “microparticle” or “microsphere,” as used herein, refer interchangeably to a particle that is about 0.1 and about 1000 μm in size.

The term, “microgel particle,” as used herein, refers to a particle comprised of gel that is about 0.1 and about 1000 μm in size.

The term “particle,” as used herein, refers to a singular unit of a larger system, such as, for example, the dermal filler system or compositions disclosed herein.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are known in the relevant field. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “PEG,” as used herein, refers to poly(ethylene glycol).

The term “PLA,” as used herein, refers to polylactic acid or polylactide.

The term “polydispersity,” as used herein is a measure of the heterogeneity of a particle of the present disclosure (e.g., microgel particles) based on size. Polydispersity may be measured by any of laser diffraction using a particle size analyzer, dynamic light scattering, small-angle X-ray scattering (SAXS), small-angle neutron scattering (SANS), or microscopy.

The term “polymer,” as used herein, refers to a class of substance composed of macromolecules comprised of monomer repeats. Non-limiting polymers include poly(ethylene glycol) (PEG), hyaluronic acid (HA), polylactic acid (PLA), collagen, collagen, poly(methylmethacrylate) (PMMA), or any combination thereof. The polymer may be synthetic, such as PEG, PLA, PMMA, and the like. The polymer may be natural, such as HA, chitosan, or proteins, such as, for example, collagen, gelatin, or lysozyme. The polymer may be a modified form of the polymer, whether it be natural or synthetic, such as for example to contain one or more thiol or vinyl derivatives disclosed herein (e.g., PEG-dithiol, 4-ARM PEG-thiol, PEG-VS, thiolated HA).

The term “pore size,” as used herein, refers the size of each individual pore in a covalently stabilized scaffold defined as interstitial void space between the particles. The pore size may be measured by approximating the void area to a circle, where the diameter of each circle may be considered the size of the pore.

The term “porosity” or “void fraction,” as used herein, refer interchangeably to a measure of the void (i.e. “empty”) spaces in a material, and may be a fraction of the volume of voids over the total volume, between 0 and 1, or may be a percentage between 0% and 100%. As an example, Porosity P=Volume_{void}/Volume_{Total}. Porosity may be measured using methods disclosed in: “Void volume fraction of granular scaffolds; Lindsay Riley, Grace Wei, Yijun Bao, Peter Cheng, Katrina L. Wilson, Yining Liu, Yiyang Gong, Tatiana Segura; bioRxiv 2022.06.14.496197; doi: https://doi.org/10.1101/2022.06.14.496197,” which is incorporated herein by reference in its entirety.

The term “precursor solution” refers to a solution of raw materials (e.g., polymers and/or peptides) used to form the microgel particles of the present disclosure.

The term “Q peptide,” as used herein, refers to a peptide comprising an amino acid sequence comprising one or more glutamine residues that serve as a substrate for an annealing agent in an annealing reaction . . . .

The term “RGD peptide,” as used herein, refers to a peptide derived from an extracellular matrix protein having an RGD motif characterized by an amino acid sequence comprising “Arg-Gly-Asp”. Non-limiting extracellular matrix proteins include fibronectin, vitronectin, fibrinogen, von Willebrand Factor, laminin, and collagen. The RGD peptide may be provided in Moral MEG, Siahaan T J., et. al. The RGD peptide may be modified for conjugation to contain a cysteine. In some embodiments, the RGD peptide comprises an amino acid sequence comprising RGDSPGERCG (SEQ ID NO: 1).

The term “storage modulus,” as used herein, refers to a mechanical property of a viscoelastic substance related to the energy that is stored in the substance, representing its elastic portion. The storage modulus represents the ratio of the elastic stress to strain. The storage modulus of microgel particles may be measured in a surrogate nonporous gel formed with the same precursor solution that is used to make the microgel particles but that is not emulsified in an oil phase to produce microspheres. Storage modulus may be measured by undergoing a measurement of shear modulus as described above, and performing an amplitude and frequency sweep of shear stress in a parallel plate system. This will enable calculation of both the storage and the loss modulus of the viscoelastic material (together the storage and loss modulus comprise the shear modulus).

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “surrogate gel” refers to a macroscopic surrogate bulk gel made from the same precursor solution used to make a microgel particles disclosed herein.

The term “tissue site,” as used herein, refers to the discrete location of a tissue where the dermal filler system disclosed herein may be delivered.

As used herein, the terms “treatment” or “treating” are used in reference to a pharmaceutical or other intervention regimen for obtaining beneficial or desired results in the recipient. Beneficial or desired results include but are not limited to a therapeutic benefit, a prophylactic benefit, or an aesthetic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. An aesthetic benefit may improve a quality of a subject's appearance. Non-limiting qualities include appearance of wrinkles or fine lines, hydration, volume, furrows, sagging skin, or other signs of aging.

The term, “VS,” as used herein, refers to vinyl sulfone.

### Systems

Dermal fillers are gel-like substances that are useful for certain therapeutic and aesthetic applications. For aesthetic applications, dermal fillers are normally injected into the skin (e.g., dermal and subdermal) to improve an aesthetic quality of the skin, such as wrinkles or fine lines, hydration, volume, furrows, sagging skin, or other signs of aging. Existing dermal fillers, such as JUVÉDERM® injectable gel fillers are made of natural polymers, like hyaluronic acid, that swell volumetrically after injection to the tissue. This swelling can be induced by electrostatic interactions between the polymers in the dermal filler and the interstitial fluid in the tissue surrounding the injection site, driving flow of the fluid into the injection and causing swelling. This volumetric swelling can lead to unintentional changes in aesthetic outcome (e.g., larger injection than intended) and actually induce unwanted tissue inflammation due to mild pressure being exerted on the tissue at the interface to the injection site. These products typically degrade in situ relatively rapidly. This class of existing dermal fillers fully degrade in situ in about 9 months, which require regular re-administration that is both painful and inconvenient for the subject. In an attempt to solve this challenge, alternative existing dermal fillers have been developed that are termed biostimulators, such as Sculptra®. These biostimulators may include stiff mechanical properties that intentionally cause a foreign body response that results in collagen deposition to fill the tissue around the injection site. Although these products result in longer volume compared to Juvéderm, they are also characterized by long-term granuloma formation and chronic inflammation. Such long-term granuloma formation and chronic inflammation can cause significant discomfort, pain, and even tissue degradation in some subjects. Further, the tissue created by this foreign body response-based mechanism is primarily scar-like and does not have the look and feel of normal tissue (e.g., it is harder and more brittle than normal tissue due to its composition, primarily of collagen-1). Therefore, there is a need for next generation dermal filler systems for treating tissue that minimizes volumetric swelling and foreign body response in the subject, while providing a lasting aesthetic or therapeutic benefit.

Disclosed herein, in some embodiments, are dermal filler systems that provide a lasting aesthetic or therapeutic effect, while minimizing the foreign body response in a subject. The dermal filler systems disclosed herein comprises microgel particles made of hydrogel with optimized degradation profiles for aesthetic applications, that contain functional groups that facilitate the annealing of the microgel particles to form a porous covalently stabilized scaffold in situ at the site of administration (e.g., an injection site). The porous covalently stabilized scaffold disclosed herein allows cells endogenous to the subject to infiltrate the pores of the scaffold and develop into a cell matrix (and eventually new tissue) that mimics tissue surrounding the scaffold. Such new tissue is not scar-like and has the look and feel of normal tissue. After complete degradation of the scaffold through endogenous degradation pathways, new tissue formed within the scaffold fills at least part of the space once occupied by the scaffold to provide lasting aesthetic quality of the subject's tissue. The dermal filler systems disclosed herein optionally contain additional agents (e.g., therapeutic agents), such as local anesthetics (e.g., lidocaine), pain medications, anti-inflammatory, antibiotics (e.g. penicillin, dicloxacillin, cephalexin) and others that can provide a therapeutic or aesthetic benefit at the site of administration. In some embodiments, the antibiotics may comprise macrolides (e.g., erythromycin, clarithromycin, dirithromycin, roxithromycin, and azithromycin), aminoglycosides (e.g., amikacin, gentamicin, neomycin, streptomycin, tobramycin), carbapenems (e.g., doripenem, meropenem), cephalosporins, tetracyclines (e.g., doxycycline, minocycline), rifamycins (e.g., rifabutin, rifampin), fluoroquinolones (e.g., ciprofloxacin, levofloxacin, delafloxacin, gemifloxacin), penicillins (e.g., amoxicillin, ampicillin, penicillin, oxacillin), oxazolidinones (e.g, linezolid, tedizolid), glycopeptides (e.g., vancomycin), polypeptides (e.g., polymyxin B, bacitracin), sulfonamides (e.g., sulfacetamide, sulfadiazine, sulfadoxine), streptogramins (e.g., quinupristin, dalfopristin). The dermal fillers disclosed herein may be formulated with one or more reagents or solvents that may improve the sterilization, stability, pH, viscosity, stiffness, porosity, degradation rate, and so forth of the dermal filler system, which can be fine-tuned depending on a given application. Tissue sites for administration of the dermal fillers disclosed herein include, but are not limited to, the midface or malar region, cheek, jawline, lips, or any combination thereof.

### Microgel Particles

Disclosed herein, in some embodiments, are dermal filler systems comprising a plurality of microgel particles. In some embodiments, the plurality of microgel particles is formed by crosslinking one or more reagents and raw materials together in accordance with various embodiments herein. The plurality of microgel particles may be in a slurry suitable for delivery to the subject by injection. Following delivery of the slurry to the subject at a tissue site, the microgel particles undergo an annealing reaction to form a porous covalently stabilized scaffold in situ. The porous covalently stabilized scaffold comprises the annealed microgel particles and may be referred to interchangeably as the “annealed particles,” “annealed microgel particles,” or “annealed scaffold.” In some embodiments, the porous covalent stabilized scaffold forms in a manner such that pores form between the microgel particles of the covalently stabilized scaffold. In some embodiments, when the dermal filler system is administered to a subject, the covalently stabilized scaffold enables growth of a cell matrix in situ at or around the tissue site of the subject that forms new tissue even after the covalently stabilized scaffold is completely degraded. In some embodiments, the new tissue that is formed is endogenous tissue of the subject. In some embodiments, the new tissue is characterized as having mature vascularization, a characteristic of surrounding tissue at the tissue site, or a combination thereof. In some embodiments, the characteristic of the surrounding tissue at the tissue site comprises functionally differentiated cell types from the surrounding tissue.

In some embodiments, the microgel particles are spherical. In some embodiments, the microgel particles are spheroidal. In some embodiments, the microgel particles are substantially spherical or substantially spheroidal. In some embodiments, the microgel particles comprise microspheres. The microgel particles may have a substantially uniform shape so as to produce pores when adjacent microgel particles are in contact with each other. Other shapes of microgel particles are contemplated, including, without limitation, oblate, prolate, round-particles, granular particles, flake particles, or 3D geometric shapes

In some embodiments, the microparticles may have a diameter or dimension (e.g., length, width, height, axis). In some embodiments, the microgel particles comprise diameters or dimensions comprising 0.1 micrometers (μm) to 1000 μm. In some embodiments, the microgel particles comprise diameters or dimensions comprising 5 micrometers (μm) to 1000 μm. In some embodiments, the diameters or dimensions comprise between 50 μm to 1000 μm. In some embodiments, the diameters or dimensions comprise between 80 μm to 140 μm. In some embodiments, the diameters comprise greater than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240,250, 260, 270, 280, 290, 300, 310, 320, 330, 340, 350, 360, 370, 380, 390, 400, 410, 420, 430, 440,450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 μm. In some embodiments, the diameters comprise less than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 μm. In some embodiments, the diameters or dimensions comprise a range of from about 5 μm to about 1100 μm. In some embodiments, the diameters or dimensions comprise a range of from about 10 μm to about 1090 μm. In some embodiments, the diameters or dimensions comprise a range of from about 15 μm to about 1080 μm. In some embodiments, the diameters or dimensions comprise a range of from about 20 μm to about 1070 μm. In some embodiments, the diameters or dimensions comprise a range of from about 25 μm to about 1060 μm. In some embodiments, the diameters or dimensions comprise a range of from about 30 μm to about 1050 μm. In some embodiments, the diameters or dimensions comprise a range of from about 35 μm to about 1040 μm. In some embodiments, the diameters or dimensions comprise a range of from about 40 μm to about 1030 μm. In some embodiments, the diameters or dimensions comprise a range of from about 45 μm to about 1020 μm. In some embodiments, the diameters or dimensions comprise a range of from about 50 μm to about 1010 μm. In some embodiments, the diameters or dimensions comprise a range of from about 55 μm to about 1000 μm. In some embodiments, the diameters or dimensions comprise a range of from about 60 μm to about 990 μm. In some embodiments, the diameters or dimensions comprise a range of from about 65 μm to about 980 μm. In some embodiments, the diameters or dimensions comprise a range of from about 70 μm to about 970 μm. In some embodiments, the diameters or dimensions comprise a range of from about 75 μm to about 960 μm. In some embodiments, the diameters or dimensions comprise a range of from about 80 μm to about 950 μm. In some embodiments, the diameters or dimensions comprise a range of from about 85 μm to about 940 μm. In some embodiments, the diameters or dimensions comprise a range of from about 90 μm to about 930 μm. In some embodiments, the diameters or dimensions comprise a range of from about 95 μm to about 920 μm. In some embodiments, the diameters or dimensions comprise a range of from about 100 μm to about 910 μm. In some embodiments, the diameters or dimensions comprise a range of from about 110 μm to about 900 μm. In some embodiments, the diameters or dimensions comprise a range of from about 120 μm to about 890 μm. In some embodiments, the diameters or dimensions comprise a range of from about 130 μm to about 880 μm. In some embodiments, the diameters or dimensions comprise a range of from about 140 μm to about 870 μm. In some embodiments, the diameters or dimensions comprise a range of from about 150 μm to about 860 μm. In some embodiments, the diameters or dimensions comprise a range of from about 160 μm to about 850 μm. In some embodiments, the diameters or dimensions comprise a range of from about 170 μm to about 840 μm. In some embodiments, the diameters or dimensions comprise a range of from about 180 μm to about 830 μm. In some embodiments, the diameters or dimensions comprise a range of from about 190 μm to about 820 μm. In some embodiments, the diameters or dimensions comprise a range of from about 200 μm to about 810 μm. In some embodiments, the diameters or dimensions comprise a range of from about 210 μm to about 800 μm. In some embodiments, the diameters or dimensions comprise a range of from about 220 μm to about 790 μm. In some embodiments, the diameters or dimensions comprise a range of from about 230 μm to about 780 μm. In some embodiments, the diameters or dimensions comprise a range of from about 240 μm to about 770 μm. In some embodiments, the diameters or dimensions comprise a range of from about 250 μm to about 760 μm. In some embodiments, the diameters or dimensions comprise a range of from about 260 μm to about 750 μm. In some embodiments, the diameters or dimensions comprise a range of from about 270 μm to about 740 μm. In some embodiments, the diameters or dimensions comprise a range of from about 280 μm to about 730 μm. In some embodiments, the diameters or dimensions comprise a range of from about 290 μm to about 720 μm. In some embodiments, the diameters or dimensions comprise a range of from about 300 μm to about 710 μm. In some embodiments, the diameters or dimensions comprise a range of from about 310 μm to about 700 μm. In some embodiments, the diameters or dimensions comprise a range of from about 320 μm to about 690 μm. In some embodiments, the diameters or dimensions comprise a range of from about 330 μm to about 680 μm. In some embodiments, the diameters or dimensions comprise a range of from about 340 μm to about 670 μm. In some embodiments, the diameters or dimensions comprise a range of from about 350 μm to about 660 μm. In some embodiments, the diameters or dimensions comprise a range of from about 360 μm to about 650 μm. In some embodiments, the diameters or dimensions comprise a range of from about 370 μm to about 640 μm. In some embodiments, the diameters or dimensions comprise a range of from about 380 μm to about 630 μm. In some embodiments, the diameters or dimensions comprise a range of from about 390 μm to about 620 μm. In some embodiments, the diameters or dimensions comprise a range of from about 400 μm to about 610 μm. In some embodiments, the diameters or dimensions comprise a range of from about 410 μm to about 600 μm. In some embodiments, the diameters or dimensions comprise a range of from about 420 μm to about 590 μm. In some embodiments, the diameters or dimensions comprise a range of from about 430 μm to about 580 μm. In some embodiments, the diameters or dimensions comprise a range of from about 440 μm to about 570 μm. In some embodiments, the diameters or dimensions comprise a range of from about 450 μm to about 560 μm. In some embodiments, the diameters or dimensions comprise a range of from about 460 μm to about 550 μm. In some embodiments, the diameters or dimensions comprise a range of from about 470 μm to about 540 μm. In some embodiments, the diameters or dimensions comprise a range of from about 480 μm to about 530 μm. In some embodiments, the diameters or dimensions comprise a range of from about 490 μm to about 520 μm. In some embodiments, the diameters or dimensions comprise a range of from about 500 μm to about 510 μm.

The microgel particles may have an average diameter or dimension of about 10 μm. The microgel particles may have an average diameter or dimension of about 15 μm. The microgel particles may have an average diameter or dimension of about 25 μm. The microgel particles may have a diameter or dimension of about 50 μm. The microgel particles may have an average diameter or dimension of about 100 μm. The microgel particles may have an average diameter or dimension of about 150 μm. The microgel particles may have an average diameter or dimension of about 200 μm. The microgel particles may have a diameter or dimension within the range of about 10 μm to about 500 μm. The microgel particles may have a diameter or dimension within the range of about 10 μm to about 200 μm. The microgel particles may have a diameter or dimension within the range of about 15 μm to about 200 μm. The microgel particles may have a diameter or dimension within the range of about 15 μm to about 150 μm. The microgel particles may have a diameter or dimension within the range of about 30 μm to about 100 μm. The microgel particles may have an average diameter or dimension of 10 μm. The microgel particles may have an average diameter or dimension of 15 μm. The microgel particles may have an average diameter or dimension of 25 μm. The microgel particles may have a diameter or dimension of 50 μm. The microgel particles may have an average diameter or dimension of 100 μm. The microgel particles may have an average diameter or dimension of 150 μm.

The microgel particles may have an average diameter or dimension of 200 μm. The microgel particles may have a diameter or dimension within the range of 10 μm to 500 μm. The microgel particles may have a diameter or dimension within the range of 10 μm to 200 μm. The microgel particles may have a diameter or dimension within the range of 15 μm to 200 μm. The microgel particles may have a diameter or dimension within the range of 15 μm to 150 μm. The microgel particles may have a diameter or dimension within the range of 30 μm to 100 μm. In some embodiments, the diameter of the microgel particle may be measured by (1) measuring the area of a microgel particle, (2) solving for the radius of the microgel particle using the equation for the area of a circle (i.e., A=pi*r\hat{ }2) and (3) solving for the diameter by multiplying the radius by 2 (i.e., D=2*r).

In some embodiments, the microgel particles comprise one or more cell adhesive peptides. In some embodiments, the cell adhesive peptide comprises at least a portion of an extracellular matrix protein. In some embodiments, the cell adhesive peptide comprises at least a portion of a collagen. In some embodiments, the cell adhesive peptide comprises at least a portion of a fibronectin. In some embodiments, the cell adhesive peptide comprises an integrin. In some embodiments, the adhesive peptide comprises a ligand to a receptor expressed on the cell. In some embodiments, the adhesive peptide comprises a cluster of differentiation (CD) protein. In some embodiments, the adhesive peptide comprises a naturally-occurring peptide. In some embodiments, the adhesive peptide comprises a synthetic peptide. In some embodiments, the cell adhesive peptide may be homologous to the naturally-occurring peptide. In some embodiments, the cell adhesive peptide comprises at least about 70% homologous to a naturally-occurring peptide. In some embodiments, the cell adhesive peptide is at least about 80% homologous to a naturally-occurring peptide. In some embodiments, the cell adhesive peptide comprises at least about 90% homology to a naturally-occurring peptide. In some embodiments, the cell adhesive peptide comprises at least 70% homology to a naturally-occurring peptide. In some embodiments, the cell adhesive peptide comprises at least 80% homology to a naturally-occurring peptide. In some embodiments, the cell adhesive peptide comprises at least 90% homology to a naturally-occurring peptide. In some embodiments, the cell adhesive peptide may be coupled to a surface of the microgel particle. In some embodiments, the cell adhesive peptides are grafted to the surface of the microgel particle. In some embodiments, the coupling may comprise one or more chemical bonds. In some embodiments, the one or more chemical bonds is one or more covalent bonds.

By way of non-limiting examples, the cell adhesive peptide may comprise an RGD peptide. In some embodiments, the RGD peptide comprises RGDSPGERCG (SEQ ID NO: 1). In some embodiments, the RGD peptide comprises ACDCRGDCFCG (SEQ ID NO: 2). In some embodiments, the RGD peptide comprises GRGDSP (SEQ ID NO: 6). In some embodiments, the RGD peptide comprises cyclo(Arg-Gly-Asp-DPhe-Val) (SEQ ID NO: 7). In some embodiments, the RGD peptide comprises cyclo(Arg-Gly-Asp-DPhe-Lys) cyclo(Arg-Gly-Asp-DPhe-Cys) (SEQ ID NO: 8). In some embodiments, the RGD peptide comprises KACDCRGDCFCG (SEQ ID NO: 9). In some embodiments, the RGD peptide comprises an amino acid sequence that is about 75% identical to an amino acid sequence provided in SEQ ID NO: 1. In some embodiments, the RGD peptide comprises an amino acid sequence that is about 85% identical to an amino acid sequence provided in SEQ ID NO: 1. In some embodiments, the RGD peptide comprises an amino acid sequence that is about 95% identical to an amino acid sequence provided in SEQ ID NO: 1. In some embodiments, the RGD peptide comprises an amino acid sequence that is about 75% identical to an amino acid sequence provided in SEQ ID NO: 2. In some embodiments, the RGD peptide comprises an amino acid sequence that is about 85% identical to an amino acid sequence provided in SEQ ID NO: 2. In some embodiments, the RGD peptide comprises an amino acid sequence that is about 95% identical to an amino acid sequence provided in SEQ ID NO: 2. In some embodiments, the RGD peptide comprises an amino acid sequence that is about 75% identical to an amino acid sequence provided in SEQ ID NO: 6. In some embodiments, the RGD peptide comprises an amino acid sequence that is about 85% identical to an amino acid sequence provided in SEQ ID NO: 6. In some embodiments, the RGD peptide comprises an amino acid sequence that is about 95% identical to an amino acid sequence provided in SEQ ID NO: 6. In some embodiments, the RGD peptide comprises an amino acid sequence that is about 75% identical to an amino acid sequence provided in SEQ ID NO: 7. In some embodiments, the RGD peptide comprises an amino acid sequence that is about 85% identical to an amino acid sequence provided in SEQ ID NO: 7. In some embodiments, the RGD peptide comprises an amino acid sequence that is about 95% identical to an amino acid sequence provided in SEQ ID NO: 7. In some embodiments, the RGD peptide comprises an amino acid sequence that is about 75% identical to an amino acid sequence provided in SEQ ID NO: 8. In some embodiments, the RGD peptide comprises an amino acid sequence that is about 85% identical to an amino acid sequence provided in SEQ ID NO: 8. In some embodiments, the RGD peptide comprises an amino acid sequence that is about 95% identical to an amino acid sequence provided in SEQ ID NO: 8. In some embodiments, the RGD peptide comprises an amino acid sequence that is about 75% identical to an amino acid sequence provided in SEQ ID NO: 9. In some embodiments, the RGD peptide comprises an amino acid sequence that is about 85% identical to an amino acid sequence provided in SEQ ID NO: 9. In some embodiments, the RGD peptide comprises an amino acid sequence that is about 95% identical to an amino acid sequence provided in SEQ ID NO: 9. In some embodiments, the RGD peptide comprises an amino acid sequence that is about 50%, 55%, 60%, 65%, 70%, 75%, 80%, 85%, 90%, 95%, 96%, 97%, 98% or 99% identical to an amino acid sequence provided in any one of SEQ ID NOS: 1-2 or 6-9. In some embodiments, the RGD peptide is provided in Moral MEG, Siahaan T J., et al., which is hereby incorporated by reference in its entirety. In some embodiments, the RGD peptide is modified to improve conjugation of the RGD peptide to a substrate, such as for example, a microgel particle disclosed herein. Non-limiting examples of modifications include addition of a cysteine residue, addition of a linker with a thiol group on one end and an amine group on the other end, a linker with a thiol group on one end and a carboxylic acid group on the other end. In some embodiments, the RGD peptide comprises a modification on either terminus (e.g., C terminus, N terminus). In some embodiments, the modification is within a flanking sequence of the RGD motif within the RGD sequence.

In some embodiments, the microgel particles comprise a polymer. In some embodiments, the polymer is or comprises a polymer backbone. In some embodiments, the polymer backbone of the polymer is comprised of the main chain of the polymer (e.g., within a substance, the polymer making up a larger proportion of the substance as compared to other polymers in the substance). In some embodiments, the main chain is a linear chain in the polymer to which any other chain may be regarded as being pendant. In some embodiments, the polymer is or comprises a co-polymer. In some embodiments, a co-polymer comprises a polymer chain comprising two or more different monomers in substantially equal proportions. In some embodiments, the polymer is capable of crosslinking and holding large amounts of water forming a water insoluble hydrogel. In some embodiments, the polymer is a natural polymer. In some embodiments, the polymer is a synthetic polymer. In some embodiments, the polymer is made of both natural and synthetic polymers. Non-limiting examples of polymer include poly(ethylene glycol), hyaluronic acid, polyacrylamide, and polymethacrylate. In some embodiments, the polymer may comprise a hydrophilic polymer, amphiphilic polymer, synthetic or natural polymer, or a copolymer of hydrophobic and hydrophilic polymers (e.g., poly(ethylene glycol) (PEG), poly(propylene glycol), poly(hydroxyethylmethacrylate), hyaluronic acid (HA), gelatin, fibrin, chitosan, heparin, heparan, and synthetic versions of HA, gelatin, fibrin, chitosan, heparin, or heparan). In some embodiments, the polymer may be made from any natural (e.g., modified HA) or synthetic polymer (e.g., PEG) capable of forming a hydrogel. In some embodiments, the polymer may comprise a natural polymer containing nitrogen, such as proteins and derivatives, including crosslinked or modified gelatins, and keratins. In some embodiments, the polymer may comprise a functional group (e.g. vinyl) at least one end of the polymer (e.g. poly(ethylene glycol) methacrylate). In some embodiments, the polymer may comprise a functional group (e.g. vinyl) incorporated into the polymer backbone (e.g. poly(methacrylate)). In some embodiments, the polymer may comprise a vinyl polymer, such as, for example: poly(ethylene glycol) acrylate, poly(ethylene glycol) methacrylate, poly(ethylene glycol) vinyl sulfone, poly(ethylene glycol) maleimide, poly(ethylene glycol) norbomene, and poly(ethylene glycol) allyl. In some embodiments, the polymer may comprise a polyacrylamide or a polymethacrylate. In some embodiments, the polymer may comprise a polyester, a polyamide, a polyurethane, or a mixture or copolymer thereof. In some embodiments, the polymer may comprise a graft copolymer obtained by initializing polymerization of a synthetic polymer on a preexisting natural polymer. In some embodiments, the polymer is or comprises hyaluronic acid (HA). In some embodiments, the polymer consists of HA. In some embodiments, the polymer is or comprises poly(ethylene) glycol (PEG). In some embodiments, the polymer consists of PEG. In some embodiments, the microgel particles comprise two or more types of polymers (e.g., polymers made of different materials). In some embodiments, the two or more types of polymers comprise HA and PEG. In some embodiments, the two or more types of polymers comprise poly(lactic acid) (PLA) and HA. In some embodiments, the two or more types of polymers comprise PLA and PEG. In some embodiments, the two or more types of polymers comprise Poly(methyl methacrylate) (PMMA) and HA. In some embodiments, the two or more types of polymers comprise PMMA and PEG. In some embodiments, the two or more types of polymers comprise a polymer comprising a functional group and a polymer that does not comprise a functional group. In some embodiments, the microgel particles comprise three or more types of polymers each independently made of a material selected from the group consisting of a hydrophilic polymer, amphiphilic polymer, synthetic or natural polymer. In some embodiments, the microgel particles comprise three or more types of polymers each independently made of a material selected from poly(ethylene glycol) (PEG), poly(lactic acid) (PLA), poly(propylene glycol), poly(hydroxyethylmethacrylate), hyaluronic acid (HA), gelatin, fibrin, chitosan, heparin, heparan, and synthetic versions of HA, gelatin, fibrin, chitosan, heparin, or heparan, or modified versions of any of these. In some embodiments, when the microgel particles comprise two or more types of polymers, the ratio of each polymer included in the microgel particles may vary. In some embodiments, the microgel particles comprise HA and PEG, and the microgel particles, in a swollen state, may comprise HA at greater than about a 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, or 20.0% weight (wt). In some embodiments, the microgel particles comprise HA and PEG, and the microgel particles, in a swollen state, may comprise HA at less than about a 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.00%, or 20.0% weight (wt). In some embodiments, the microgel particles comprise HA and PEG, and the microgel particles, in a swollen state, may comprise HA at about a 0.01% to about a 20.0% wt range. In some embodiments, the microgel particles comprise HA and PEG, and the microgel particles, in a swollen state, may comprise HA at about a 0.01% to about a 19.0% wt range. In some embodiments, the microgel particles comprise HA and PEG, and the microgel particles, in a swollen state, may comprise HA at about a 0.01% to about a 18.0% wt range. In some embodiments, the microgel particles comprise HA and PEG, and the microgel particles, in a swollen state, may comprise HA at about a 0.01% to about a 17.0% wt range. In some embodiments, the microgel particles comprise HA and PEG, and the microgel particles, in a swollen state, may comprise HA at about a 0.01% to about a 16.0% wt range. In some embodiments, the microgel particles comprise HA and PEG, and the microgel particles, in a swollen state, may comprise HA at about a 0.01% to about a 15.0% wt range. In some embodiments, the microgel particles comprise HA and PEG, and the microgel particles, in a swollen state, may comprise HA at about a 0.01% to about a 14.0% wt range. In some embodiments, the microgel particles comprise HA and PEG, and the microgel particles, in a swollen state, may comprise HA at about a 0.01% to about a 13.0% wt range. In some embodiments, the microgel particles comprise HA and PEG, and the microgel particles, in a swollen state, may comprise HA at about a 0.01% to about a 12.0% wt range. In some embodiments, the microgel particles comprise HA and PEG, and the microgel particles, in a swollen state, may comprise HA at about a 0.01% to about a 11.0% wt range. In some embodiments, the microgel particles comprise HA and PEG, and the microgel particles, in a swollen state, may comprise HA at about a 0.01% to about a 10.0% wt range. In some embodiments, the microgel particles comprise HA and PEG, and the microgel particles, in a swollen state, may comprise HA at about a 0.010% to about a 9.0% wt range. In some embodiments, the microgel particles comprise HA and PEG, and the microgel particles, in a swollen state, may comprise HA at about a 0.01% to about a 8.0% wt range. In some embodiments, the microgel particles comprise HA and PEG, and the microgel particles, in a swollen state, may comprise HA at about a 0.01% to about a 7.0% wt range. In some embodiments, the microgel particles comprise HA and PEG, and the microgel particles, in a swollen state, may comprise HA at about a 0.01% to about a 6.0% wt range. In some embodiments, the microgel particles comprise HA and PEG, and the microgel particles, in a swollen state, may comprise HA at about a 0.01% to about a 5.0% wt range. In some embodiments, the microgel particles comprise HA and PEG, and the microgel particles, in a swollen state, may comprise HA at about a 0.01% to about a 4.0% wt range. In some embodiments, the microgel particles comprise HA and PEG, and the microgel particles, in a swollen state, may comprise HA at about a 0.010% to about a 3.0% wt range. In some embodiments, the microgel particles comprise HA and PEG, and the microgel particles, in a swollen state, may comprise HA at about a 0.01% to about a 2.0% wt range. In some embodiments, the microgel particles comprise HA and PEG, and the microgel particles, in a swollen state, may comprise HA at about a 0.01% to about a 1.0% wt range. In some embodiments, the microgel particles comprise HA and PEG, and the microgel particles, in a swollen state, may comprise HA at about a 0.01% to about a 0.95% wt range. In some embodiments, the microgel particles comprise HA and PEG, and the microgel particles, in a swollen state, may comprise HA at about a 0.01% to about a 0.90% wt range. In some embodiments, the microgel particles comprise HA and PEG, and the microgel particles, in a swollen state, may comprise HA at about a 0.01% to about a 0.85% wt range. In some embodiments, the microgel particles comprise HA and PEG, and the microgel particles, in a swollen state, may comprise HA at about a 0.01% to about a 0.80% wt range. In some embodiments, the microgel particles comprise HA and PEG, and the microgel particles, in a swollen state, may comprise HA at about a 0.01% to about a 0.75% wt range. In some embodiments, the microgel particles comprise HA and PEG, and the microgel particles, in a swollen state, may comprise HA at about a 0.01% to about a 0.70% wt range. In some embodiments, the microgel particles comprise HA and PEG, and the microgel particles, in a swollen state, may comprise HA at about a 0.01% to about a 0.65% wt range. In some embodiments, the microgel particles comprise HA and PEG, and the microgel particles, in a swollen state, may comprise HA at about a 0.01% to about a 0.60% wt range. In some embodiments, the microgel particles comprise HA and PEG, and the microgel particles, in a swollen state, may comprise HA at about a 0.01% to about a 0.55% wt range. In some embodiments, the microgel particles comprise HA and PEG, and the microgel particles, in a swollen state, may comprise HA at about a 0.01% to about a 0.50% wt range. In some embodiments, the microgel particles comprise HA and PEG, and the microgel particles, in a swollen state, may comprise HA at about a 0.01% to about a 0.45% wt range. In some embodiments, the microgel particles comprise HA and PEG, and the microgel particles, in a swollen state, may comprise HA at about a 0.01% to about a 0.40% wt range. In some embodiments, the microgel particles comprise HA and PEG, and the microgel particles, in a swollen state, may comprise HA at about a 0.01% to about a 0.35% wt range. In some embodiments, the microgel particles comprise HA and PEG, and the microgel particles, in a swollen state, may comprise HA at about a 0.01% to about a 0.30% wt range. In some embodiments, the microgel particles comprise HA and PEG, and the microgel particles, in a swollen state, may comprise HA at about a 0.01% to about a 0.25% wt range. In some embodiments, the microgel particles comprise HA and PEG, and the microgel particles, in a swollen state, may comprise HA at about a 0.01% to about a 0.20% wt range. In some embodiments, the microgel particles comprise HA and PEG, and the microgel particles, in a swollen state, may comprise HA at about a 0.01% to about a 0.15% wt range. In some embodiments, the microgel particles comprise HA and PEG, and the microgel particles, in a swollen state, may comprise HA at about a 0.01% to about a 0.10% wt range. In some embodiments, the microgel particles comprise HA and PEG, and the microgel particles, in a swollen state, may comprise HA at about a 0.01% to about a 0.09% wt range. In some embodiments, the microgel particles comprise HA and PEG, and the microgel particles, in a swollen state, may comprise HA at about a 0.01% to about a 0.08% wt range. In some embodiments, the microgel particles comprise HA and PEG, and the microgel particles, in a swollen state, may comprise HA at about a 0.01% to about a 0.07% wt range. In some embodiments, the microgel particles comprise HA and PEG, and the microgel particles, in a swollen state, may comprise HA at about a 0.01% to about a 0.06% wt range. In some embodiments, the microgel particles comprise HA and PEG, and the microgel particles, in a swollen state, may comprise HA at about a 0.01% to about a 0.05% wt range. In some embodiments, the microgel particles comprise HA and PEG, and the microgel particles, in a swollen state, may comprise PEG at greater than about a 0.10%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, or 20.0% weight (wt). In some embodiments, the microgel particles comprise HA and PEG, and the microgel particles, in a swollen state, may comprise PEG at less than about a 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, or 20.0% weight (wt). In some embodiments, the microgel particles comprise HA and PEG, and the microgel particles, in a swollen state, may comprise PEG at about a 0.10% to about a 20.0% wt range. In some embodiments, the microgel particles comprise HA and PEG, and the microgel particles, in a swollen state, may comprise PEG at about a 0.1% to about a 19.0% wt range. In some embodiments, the microgel particles comprise HA and PEG, and the microgel particles, in a swollen state, may comprise PEG at about a 0.1% to about a 18.0% wt range. In some embodiments, the microgel particles comprise HA and PEG, and the microgel particles, in a swollen state, may comprise PEG at about a 0.1% to about a 17.0% wt range. In some embodiments, the microgel particles comprise HA and PEG, and the microgel particles, in a swollen state, may comprise PEG at about a 0.1% to about a 16.0% wt range. In some embodiments, the microgel particles comprise HA and PEG, and the microgel particles, in a swollen state, may comprise PEG at about a 0.1% to about a 15.0% wt range. In some embodiments, the microgel particles comprise HA and PEG, and the microgel particles, in a swollen state, may comprise PEG at about a 0.1% to about a 14.0% wt range. In some embodiments, the microgel particles comprise HA and PEG, and the microgel particles, in a swollen state, may comprise PEG at about a 0.1% to about a 13.0% wt range. In some embodiments, the microgel particles comprise HA and PEG, and the microgel particles, in a swollen state, may comprise PEG at about a 0.1% to about a 12.0% wt range. In some embodiments, the microgel particles comprise HA and PEG, and the microgel particles, in a swollen state, may comprise PEG at about a 0.1% to about a 11.0% wt range. In some embodiments, the microgel particles comprise HA and PEG, and the microgel particles, in a swollen state, may comprise PEG at about a 0.1% to about a 10.0% wt range. In some embodiments, the microgel particles comprise HA and PEG, and the microgel particles, in a swollen state, may comprise PEG at about a 0.1% to about a 9.0% wt range. In some embodiments, the microgel particles comprise HA and PEG, and the microgel particles, in a swollen state, may comprise PEG at about a 0.1% to about a 8.0% wt range. In some embodiments, the microgel particles comprise HA and PEG, and the microgel particles, in a swollen state, may comprise PEG at about a 0.1% to about a 7.0% wt range. In some embodiments, the microgel particles comprise HA and PEG, and the microgel particles, in a swollen state, may comprise PEG at about a 0.1% to about a 6.0% wt range. In some embodiments, the microgel particles comprise HA and PEG, and the microgel particles, in a swollen state, may comprise PEG at about a 0.1% to about a 5.0% wt range. In some embodiments, the microgel particles comprise HA and PEG, and the microgel particles, in a swollen state, may comprise PEG at about a 0.1% to about a 4.0% wt range. In some embodiments, the microgel particles comprise HA and PEG, and the microgel particles, in a swollen state, may comprise PEG at about a 0.1% to about a 3.0% wt range. In some embodiments, the microgel particles comprise HA and PEG, and the microgel particles, in a swollen state, may comprise PEG at about a 0.1% to about a 2.0% wt range. In some embodiments, the microgel particles comprise HA and PEG, and the microgel particles, in a swollen state, may comprise PEG at about a 0.1% to about a 1.0% wt range. In some embodiments, the microgel particles comprise HA and PEG, and the microgel particles, in a swollen state, may comprise PEG at about a 0.1% to about a 0.95% wt range. In some embodiments, the microgel particles comprise HA and PEG, and the microgel particles, in a swollen state, may comprise PEG at about a 0.1% to about a 0.90% wt range. In some embodiments, the microgel particles comprise HA and PEG, and the microgel particles, in a swollen state, may comprise PEG at about a 0.1% to about a 0.85% wt range. In some embodiments, the microgel particles comprise HA and PEG, and the microgel particles, in a swollen state, may comprise PEG at about a 0.1% to about a 0.80% wt range. In some embodiments, the microgel particles comprise HA and PEG, and the microgel particles, in a swollen state, may comprise PEG at about a 0.1% to about a 0.75% wt range. In some embodiments, the microgel particles comprise HA and PEG, and the microgel particles, in a swollen state, may comprise PEG at about a 0.1% to about a 0.70% wt range. In some embodiments, the microgel particles comprise HA and PEG, and the microgel particles, in a swollen state, may comprise PEG at about a 0.1% to about a 0.65% wt range. In some embodiments, the microgel particles comprise HA and PEG, and the microgel particles, in a swollen state, may comprise PEG at about a 0.1% to about a 0.60% wt range. In some embodiments, the microgel particles comprise HA and PEG, and the microgel particles, in a swollen state, may comprise PEG at about a 0.1% to about a 0.55% wt range. In some embodiments, the microgel particles comprise HA and PEG, and the microgel particles, in a swollen state, may comprise PEG at about a 0.1% to about a 0.50% wt range. In some embodiments, the microgel particles comprise HA and PEG, and the microgel particles, in a swollen state, may comprise PEG at about a 0.1% to about a 0.45% wt range. In some embodiments, the microgel particles comprise HA and PEG, and the microgel particles, in a swollen state, may comprise PEG at about a 0.1% to about a 0.40% wt range. In some embodiments, the microgel particles comprise HA and PEG, and the microgel particles, in a swollen state, may comprise PEG at about a 0.1% to about a 0.35% wt range. In some embodiments, the microgel particles comprise HA and PEG, and the microgel particles, in a swollen state, may comprise PEG at about a 0.1% to about a 0.30% wt range. In some embodiments, the microgel particles comprise HA and PEG, and the microgel particles, in a swollen state, may comprise PEG at about a 0.1% to about a 0.25% wt range. In some embodiments, the microgel particles comprise HA and PEG, and the microgel particles, in a swollen state, may comprise PEG at about a 0.1% to about a 0.20% wt range. In some embodiments, the microgel particles comprise a hydrogel. In some embodiments, the hydrogel comprises one or more of the polymers disclosed herein. For example, a PEG hydrogel is a water insoluble hydrogel made of PEG that is capable of holding water. In another example, an HA hydrogel is a water insoluble hydrogel made of HA that is capable of holding water. Likewise, hydrogel microgel particles made of a copolymer of HA and PEG is a water insoluble hydrogel made of HA and PEG that is capable of holding water. In some embodiments, the first polymer and the second polymer are a copolymer having approximately identical molecular weights of each polymer. In some embodiments, the copolymer comprises HA and PEG. In some embodiments, the copolymer consists of HA and PEG.

In some embodiments, the microgel particles comprise, alternatively or in addition to the polymer, a support material. In some embodiments, the support material is suitable for tissue engineering or regenerative medicine applications. In some embodiments, the support material is biocompatible. In some embodiments, the support material is biodegradable. Examples of support material include, but are not limited to, natural polymeric carbohydrates and their synthetically modified, crosslinked, or substituted derivatives, such as gelatin, agar, agarose, crosslinked alginic acid, chitin, substituted and crosslinked guar gums, cellulose esters, especially with nitrous acids and carboxylic acids, mixed cellulose esters, and cellulose ethers; natural polymers containing nitrogen, such as proteins and derivatives, including crosslinked or modified gelatins, heparin, chrondriotin sulfate, glycosaminoglycans, and keratins; vinyl polymers such as poly(ethylene glycol)acrylate/methacrylate/vinyl sulfone/maleimide/norbomene/allyl, polyacrylamides, polymethacrylates, copolymers and terpolymers of the above polycondensates, such as polyesters, polyamides, and other polymers, such as polyurethanes; and mixtures or copolymers of the above classes, such as graft copolymers obtained by initializing polymerization of synthetic polymers on a preexisting natural polymer. A variety of biocompatible and biodegradable polymers are available for use in therapeutic applications; examples include: polycaprolactone, polyglycolide, polylactide, poly(lactic-co-glycolic acid) (PLGA), and poly-3-hydroxybutyrate.

In some embodiments, the polymer (e.g., copolymer (e.g., HA and PEG)) may be present in the microgel particles, the dermal filler system containing the microgel particles, the resulting covalently stabilized scaffold, or any combination thereof at about 1% weight (% wt) to about a 50% wt. In some embodiments, the polymer may be present in the microgel particles, the dermal filler system containing the microgel particles, the resulting covalently stabilized scaffold, or any combination thereof at greater than or equal to about 1% wt, 5% wt, 10% wt, 15% wt, 20% wt, 25% wt, 30% wt, 35% wt, 40% wt, 45% wt, or 50% wt. In some embodiments, the polymer may be present at less than or equal to about 1% wt, 5% wt, 10% wt, 15% wt, 20% wt, 25% wt, 30% wt, 35% wt, 40% wt, 45% wt, or 50% wt. In some embodiments, the polymer may be present at greater than or equal to about 0.1% wt, 0.2% wt, 0.3% wt, 0.4% wt, 0.5% wt, 0.6% wt, 0.7% wt, 0.8% wt, 0.9% wt, 1% wt, 1.1% wt, 1.2% wt, 1.3% wt, 1.4% wt, 1.5% wt, 1.6% wt, 1.7% wt, 1.8% wt, 1.9% wt, 2.0% wt, 2.5% wt, 3% wt, 4% wt or 5% wt. In some embodiments, the polymer may be present at less than or equal to about 0.1% wt, 0.2% wt, 0.3% wt, 0.4% wt, 0.5% wt, 0.6% wt, 0.7% wt, 0.8% wt, 0.9% wt, 1% wt, 1.1% wt, 1.2% wt, 1.3% wt, 1.4% wt, 1.5% wt, 1.6% wt, 1.7% wt, 1.8% wt, 1.9% wt, 2.0% wt, 2.5% wt, 3% wt, 4% wt or 5% wt. In some embodiments, the polymer may be present at about 5% weight (% wt) to about a 45% wt. In some embodiments, the polymer may be present at about 10% weight (% wt) to about a 40% wt. In some embodiments, the polymer may be present at about 15% weight (% wt) to about a 35% wt. In some embodiments, the polymer may be present at about 20% weight (% wt) to about a 30% wt. In some embodiments, the polymer may be present at about 0.1% wt to about 1.5% wt. In some embodiments, the polymer may be present at about 0.5% wt.

In some embodiments, the polymer is modified relative to an otherwise identical polymer that does not contain a modification. In some embodiments, the modified polymer is a modified HA, such as for example, HA modified to contain a thiol or derivative thereof. In some embodiments, the modified polymer comprises modified PEG, such as for example PEG modified to contain a thiol or derivative thereof. Non-limiting modifications include thiolation of the carboxylic acid group on the HA polymer, thiolation of the primary alcohol group on the HA polymer, and thiolation of the terminal alcohol group on the PEG polymer.

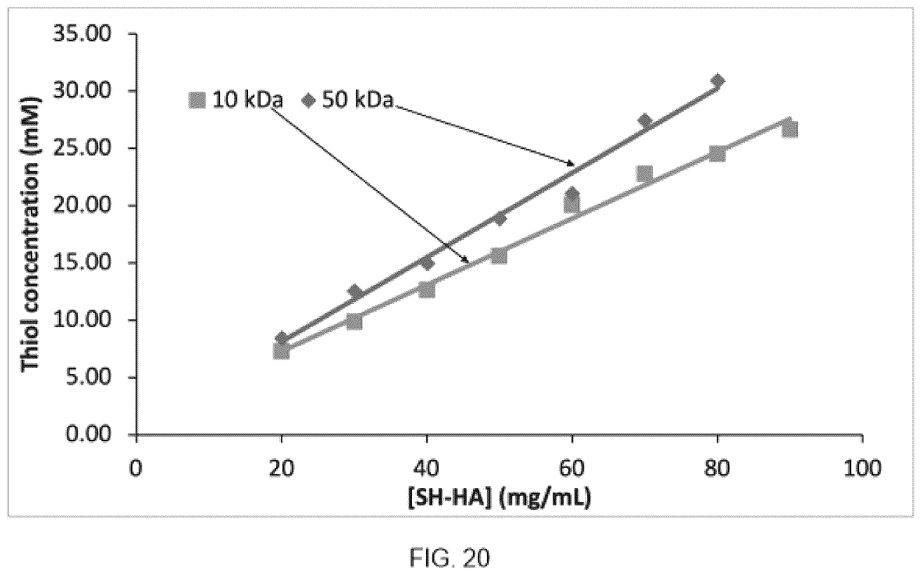

In some embodiments, the molecular weights of the polymers may have an effect on the properties of the microgel particles, the dermal filler system containing the microgel particles, the resulting covalently stabilized scaffold, or any combination thereof. For example, as discussed below in Example 3, the molecular weight of HA may have an effect on the concentration of one or more functional groups disclosed elsewhere herein (e.g., thiols) of the microgel particles. As a non-limiting example, a higher molecular weight HA can lead to a higher thiol concentration in the dermal filler system at a fixed weight percent concentration of the HA itself, as shown in FIG. 20.

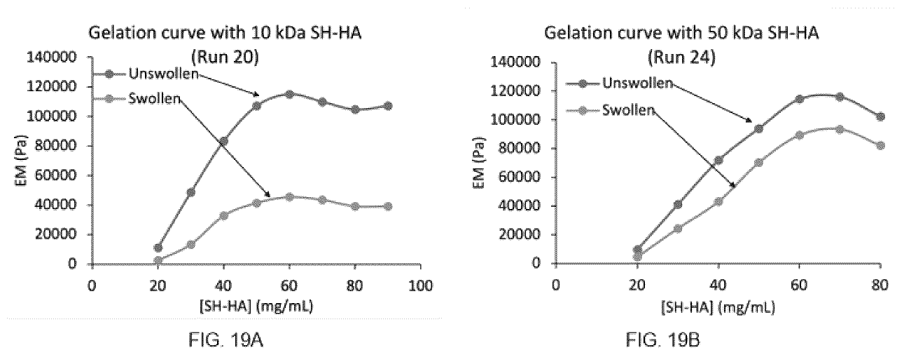

In some embodiments, the molecular weight and concentration of the modified HA can be adjusted to achieve a desired mechanical property of the hydrogel (including elastic compressive modulus and/or storage modulus) at a fixed concentration of modified PEG. As shown in FIGS. 19A-19B, HA of higher molecular weight can be used to achieve higher compressive elastic moduli for swollen particles. In some embodiments, the higher compressive elastic moduli may be as a result of less swelling due to an increase in the crosslinking density of the particles. In some embodiments, the molecular weight and concentration of the modified PEG can be adjusted to achieve a desired mechanical property of the hydrogel (including elastic compressive modulus and/or storage modulus) at a fixed concentration of modified HA.

In some embodiments, the HA comprises a molecular weight of about 1 kilodalton (kDa) to about 1 megadalton (1 MDa). In some embodiments, the HA comprises a molecular weight of about 10 kDa to 250 kDa (e.g., 10, 40, 50, 150, and 250 kDa). In some embodiments, the HA comprises a molecular weight of greater than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 kDa. In some embodiments, the HA comprises a molecular weight of less than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240,250, 260, 270, 280, 290, 300, 310, 320, 330, 340, 350, 360, 370, 380, 390, 400, 410, 420, 430, 440,450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 kDa. In some embodiments, the HA comprises a molecular weight range of from about 5 kDa to about 1100 kDa. In some embodiments, the HA comprises a molecular weight range of from about 10 kDa to about 1090 kDa. In some embodiments, the HA comprises a molecular weight range of from about 15 kDa to about 1080 kDa. In some embodiments, the HA comprises a molecular weight range of from about 20 kDa to about 1070 kDa. In some embodiments, the HA comprises a molecular weight range of from about 25 kDa to about 1060 kDa. In some embodiments, the HA comprises a molecular weight range of from about 30 kDa to about 1050 kDa. In some embodiments, the HA comprises a molecular weight range of from about 35 kDa to about 1040 kDa. In some embodiments, the HA comprises a molecular weight range of from about 40 kDa to about 1030 kDa. In some embodiments, the HA comprises a molecular weight range of from about 45 kDa to about 1020 kDa. In some embodiments, the HA comprises a molecular weight range of from about 50 kDa to about 1010 kDa. In some embodiments, the HA comprises a molecular weight range of from about 55 kDa to about 1000 kDa. In some embodiments, the HA comprises a molecular weight range of from about 60 kDa to about 990 kDa. In some embodiments, the HA comprises a molecular weight range of from about 65 kDa to about 980 kDa. In some embodiments, the HA comprises a molecular weight range of from about 70 kDa to about 970 kDa. In some embodiments, the HA comprises a molecular weight range of from about 75 kDa to about 960 kDa. In some embodiments, the HA comprises a molecular weight range of from about 80 kDa to about 950 kDa. In some embodiments, the HA comprises a molecular weight range of from about 85 kDa to about 940 kDa. In some embodiments, the HA comprises a molecular weight range of from about 90 kDa to about 930 kDa. In some embodiments, the HA comprises a molecular weight range of from about 95 kDa to about 920 kDa. In some embodiments, the HA comprises a molecular weight range of from about 100 kDa to about 910 kDa. In some embodiments, the HA comprises a molecular weight range of from about 110 kDa to about 900 kDa. In some embodiments, the HA comprises a molecular weight range of from about 120 kDa to about 890 kDa. In some embodiments, the HA comprises a molecular weight range of from about 130 kDa to about 880 kDa. In some embodiments, the HA comprises a molecular weight range of from about 140 kDa to about 870 kDa. In some embodiments, the HA comprises a molecular weight range of from about 150 kDa to about 860 kDa. In some embodiments, the HA comprises a molecular weight range of from about 160 kDa to about 850 kDa. In some embodiments, the HA comprises a molecular weight range of from about 170 kDa to about 840 kDa. In some embodiments, the HA comprises a molecular weight range of from about 180 kDa to about 830 kDa. In some embodiments, the HA comprises a molecular weight range of from about 190 kDa to about 820 kDa. In some embodiments, the HA comprises a molecular weight range of from about 200 kDa to about 810 kDa. In some embodiments, the HA comprises a molecular weight range of from about 210 kDa to about 800 kDa. In some embodiments, the HA comprises a molecular weight range of from about 220 kDa to about 790 kDa. In some embodiments, the HA comprises a molecular weight range of from about 230 kDa to about 780 kDa. In some embodiments, the HA comprises a molecular weight range of from about 240 kDa to about 770 kDa. In some embodiments, the HA comprises a molecular weight range of from about 250 kDa to about 760 kDa. In some embodiments, the HA comprises a molecular weight range of from about 260 kDa to about 750 kDa. In some embodiments, the HA comprises a molecular weight range of from about 270 kDa to about 740 kDa. In some embodiments, the HA comprises a molecular weight range of from about 280 kDa to about 730 kDa. In some embodiments, the HA comprises a molecular weight range of from about 290 kDa to about 720 kDa. In some embodiments, the HA comprises a molecular weight range of from about 300 kDa to about 710 kDa. In some embodiments, the HA comprises a molecular weight range of from about 310 kDa to about 700 kDa. In some embodiments, the HA comprises a molecular weight range of from about 320 kDa to about 690 kDa. In some embodiments, the HA comprises a molecular weight range of from about 330 kDa to about 680 kDa. In some embodiments, the HA comprises a molecular weight range of from about 340 kDa to about 670 kDa. In some embodiments, the HA comprises a molecular weight range of from about 350 kDa to about 660 kDa. In some embodiments, the HA comprises a molecular weight range of from about 360 kDa to about 650 kDa. In some embodiments, the HA comprises a molecular weight range of from about 370 kDa to about 640 kDa. In some embodiments, the HA comprises a molecular weight range of from about 380 kDa to about 630 kDa. In some embodiments, the HA comprises a molecular weight range of from about 390 kDa to about 620 kDa. In some embodiments, the HA comprises a molecular weight range of from about 400 kDa to about 610 kDa. In some embodiments, the HA comprises a molecular weight range of from about 410 kDa to about 600 kDa. In some embodiments, the HA comprises a molecular weight range of from about 420 kDa to about 590 kDa. In some embodiments, the HA comprises a molecular weight range of from about 430 kDa to about 580 kDa. In some embodiments, the HA comprises a molecular weight range of from about 440 kDa to about 570 kDa. In some embodiments, the HA comprises a molecular weight range of from about 450 kDa to about 560 kDa. In some embodiments, the HA comprises a molecular weight range of from about 460 kDa to about 550 kDa. In some embodiments, the HA comprises a molecular weight range of from about 470 kDa to about 540 kDa. In some embodiments, the HA comprises a molecular weight range of from about 480 kDa to about 530 kDa. In some embodiments, the HA comprises a molecular weight range of from about 490 kDa to about 520 kDa. In some embodiments, the HA comprises a molecular weight range of from about 500 kDa to about 510 kDa.

In some embodiments, the PEG comprises a molecular weight of about 1 kilodalton (kDa) to about 5 kDa. In some embodiments, the PEG comprises a molecular weight of about 1, 2, 3, 4, 5, 6, 7, 8, 9, or 10 kDa. In some embodiments, the PEG comprises a molecular weight of about of 1 kilodalton (kDa) to 1000 kDa. In some embodiments, the PEG comprises a molecular weight of greater than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 kDa. In some embodiments, the PEG comprises a molecular weight of less than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 kDa. In some embodiments, the PEG comprises a molecular weight range of from about 5 kDa to about 1100 kDa. In some embodiments, the PEG comprises a molecular weight range of from about 10 kDa to about 1090 kDa. In some embodiments, the PEG comprises a molecular weight range of from about 15 kDa to about 1080 kDa. In some embodiments, the PEG comprises a molecular weight range of from about 20 kDa to about 1070 kDa. In some embodiments, the PEG comprises a molecular weight range of from about 25 kDa to about 1060 kDa. In some embodiments, the PEG comprises a molecular weight range of from about 30 kDa to about 1050 kDa. In some embodiments, the PEG comprises a molecular weight range of from about 35 kDa to about 1040 kDa. In some embodiments, the PEG comprises a molecular weight range of from about 40 kDa to about 1030 kDa. In some embodiments, the PEG comprises a molecular weight range of from about 45 kDa to about 1020 kDa. In some embodiments, the PEG comprises a molecular weight range of from about 50 kDa to about 1010 kDa. In some embodiments, the PEG comprises a molecular weight range of from about 55 kDa to about 1000 kDa. In some embodiments, the PEG comprises a molecular weight range of from about 60 kDa to about 990 kDa. In some embodiments, the PEG comprises a molecular weight range of from about 65 kDa to about 980 kDa. In some embodiments, the PEG comprises a molecular weight range of from about 70 kDa to about 970 kDa. In some embodiments, the PEG comprises a molecular weight range of from about 75 kDa to about 960 kDa. In some embodiments, the PEG comprises a molecular weight range of from about 80 kDa to about 950 kDa. In some embodiments, the PEG comprises a molecular weight range of from about 85 kDa to about 940 kDa. In some embodiments, the PEG comprises a molecular weight range of from about 90 kDa to about 930 kDa. In some embodiments, the PEG comprises a molecular weight range of from about 95 kDa to about 920 kDa. In some embodiments, the PEG comprises a molecular weight range of from about 100 kDa to about 910 kDa. In some embodiments, the PEG comprises a molecular weight range of from about 110 kDa to about 900 kDa. In some embodiments, the PEG comprises a molecular weight range of from about 120 kDa to about 890 kDa. In some embodiments, the PEG comprises a molecular weight range of from about 130 kDa to about 880 kDa. In some embodiments, the PEG comprises a molecular weight range of from about 140 kDa to about 870 kDa. In some embodiments, the PEG comprises a molecular weight range of from about 150 kDa to about 860 kDa. In some embodiments, the PEG comprises a molecular weight range of from about 160 kDa to about 850 kDa. In some embodiments, the PEG comprises a molecular weight range of from about 170 kDa to about 840 kDa. In some embodiments, the PEG comprises a molecular weight range of from about 180 kDa to about 830 kDa. In some embodiments, the PEG comprises a molecular weight range of from about 190 kDa to about 820 kDa. In some embodiments, the PEG comprises a molecular weight range of from about 200 kDa to about 810 kDa. In some embodiments, the PEG comprises a molecular weight range of from about 210 kDa to about 800 kDa. In some embodiments, the PEG comprises a molecular weight range of from about 220 kDa to about 790 kDa. In some embodiments, the PEG comprises a molecular weight range of from about 230 kDa to about 780 kDa. In some embodiments, the PEG comprises a molecular weight range of from about 240 kDa to about 770 kDa. In some embodiments, the PEG comprises a molecular weight range of from about 250 kDa to about 760 kDa. In some embodiments, the PEG comprises a molecular weight range of from about 260 kDa to about 750 kDa. In some embodiments, the PEG comprises a molecular weight range of from about 270 kDa to about 740 kDa. In some embodiments, the PEG comprises a molecular weight range of from about 280 kDa to about 730 kDa. In some embodiments, the PEG comprises a molecular weight range of from about 290 kDa to about 720 kDa. In some embodiments, the PEG comprises a molecular weight range of from about 300 kDa to about 710 kDa. In some embodiments, the PEG comprises a molecular weight range of from about 310 kDa to about 700 kDa. In some embodiments, the PEG comprises a molecular weight range of from about 320 kDa to about 690 kDa. In some embodiments, the PEG comprises a molecular weight range of from about 330 kDa to about 680 kDa. In some embodiments, the PEG comprises a molecular weight range of from about 340 kDa to about 670 kDa. In some embodiments, the PEG comprises a molecular weight range of from about 350 kDa to about 660 kDa. In some embodiments, the PEG comprises a molecular weight range of from about 360 kDa to about 650 kDa. In some embodiments, the PEG comprises a molecular weight range of from about 370 kDa to about 640 kDa. In some embodiments, the PEG comprises a molecular weight range of from about 380 kDa to about 630 kDa. In some embodiments, the PEG comprises a molecular weight range of from about 390 kDa to about 620 kDa. In some embodiments, the PEG comprises a molecular weight range of from about 400 kDa to about 610 kDa. In some embodiments, the PEG comprises a molecular weight range of from about 410 kDa to about 600 kDa. In some embodiments, the PEG comprises a molecular weight range of from about 420 kDa to about 590 kDa. In some embodiments, the PEG comprises a molecular weight range of from about 430 kDa to about 580 kDa. In some embodiments, the PEG comprises a molecular weight range of from about 440 kDa to about 570 kDa. In some embodiments, the PEG comprises a molecular weight range of from about 450 kDa to about 560 kDa. In some embodiments, the PEG comprises a molecular weight range of from about 460 kDa to about 550 kDa. In some embodiments, the PEG comprises a molecular weight range of from about 470 kDa to about 540 kDa. In some embodiments, the PEG comprises a molecular weight range of from about 480 kDa to about 530 kDa. In some embodiments, the PEG comprises a molecular weight range of from about 490 kDa to about 520 kDa. In some embodiments, the PEG comprises a molecular weight range of from about 500 kDa to about 510 kDa.

In some embodiments, the microgel particles can be functionalized to comprise one or more functional groups. For example, a microgel particle made of hydrogel may be functionalized to comprise a functional group coupled thereto. In some embodiments, the functional group comprises a hydroxyl functional group, methyl functional group, carbonyl functional group, carboxyl functional group, amino functional group, phosphate functional group, sulfhydryl functional group, or a combination thereof. In some embodiments, the functional group comprises alkanes, alkenes, alkynes, ethers, sulfides, amines, aldehydes, ketones, imines, nitriles, or a combination thereof. In some embodiments, the functional group may be coupled to the microgel particle using a bond, linkage, interaction, or other coupling mechanism. In some embodiments, the bond is a covalent bond. In some embodiments, the bond is a non-covalent bond. In some embodiments, the bond is selected from a carbon-carbon bond, an amide bond, an imine bond, an ester bond, a thioether bond, a disulfide bond, a hydrazone bond, a hydrogen bond, and a metal ligand bond. In some embodiments, the ester bond comprises a cyclic boronate ester. In some embodiments, the linkage is selected from a carbamate linkage, an ester linkage, and a thioether linkage. In some embodiments, the coupling is selected from an oxime coupling, and a thiourea coupling. In some embodiments, the interaction is selected from an electrostatic interaction and a van der Waals interaction. In some embodiments, the functional group comprises a thiol or a derivative thereof. In some embodiments, the functional group comprises matrix metalloproteinase (MMP)-sensitive peptide. Non-limiting examples of thiol derivatives include any organosulfur compound of the form R—SH, where R represents an alkyl or other organic substituent. In some embodiments, the thiol derivatives include: methanethiol, ethanethiol, 1-propanethiol, 2-propoanethiol, allyl mercaptan, butanethiol, tert-butyl mercaptan, pentanethiols, thiophenol, dimercaptosuccinic acid, thioacetic acid, coenzyme A, glutathione, metallothionein, cysteine, 2-mercaptoethanol, dithiothreitol, dithioerythritol, 1-mercaptoindole, grapefruit mercaptan, furan-2-ylmethanethiol, 3-mercaptopropane-1, 2-diol, 3-mercapto-1-propanesulfonic acid, 1-hexadecanethiol, pentachlorobenzenethiol, or a combination thereof. In some embodiments, the functional group comprises a vinyl or a derivative thereof. In some embodiments, the functional group comprises a vinyl sulfone (VS) or a derivative thereof. Non-limiting examples of vinyl derivatives include alkenes comprising ethenes, propenes, butenes, pentenes, hexenes, heptenes, octenes, acrylate, methacrylate, acrylamide, methacrylamide, maleimide, norbornene, or a combination thereof. Non-limiting examples of VS derivatives include phenyl vinyl sulfone, methyl vinyl sulfone, ethyl vinyl sulfone, or any combination thereof. In some embodiments, the functional group comprises a thiol and VS, or derivatives of either the thiol or the VS. In some embodiments, the HA is modified to comprise the thiol or the derivative thereof to form thiolated-HA (e.g., SH-HA). In some embodiments, the PEG is modified to comprise the VS or the derivative thereof to form PEG-VS. In some embodiments, the HA is modified to comprise the VS or the derivative thereof to form HA-VS. In some embodiments, the PEG is modified to comprise the thiol or the derivative thereof to form thiolated PEG (e.g., PEG-SH, PEG-dithiol). In some embodiments, the PEG-VS comprises a multi-arm PEG-VS. In some embodiments, the multi-arm PEG-VS comprises, 4-arm or 6-arm or 8-arm PEG-VS. In some embodiments, the multi-arm PEG-VS comprises star-shaped polymer, brushed polymer, branched polymer, comb polymer or dendritic polymer PEG-VS. In some embodiments, the VS comprises vinyl sulfone.

In some embodiments, the microgel particles comprise functional groups that may be pH responsive (e.g., pH responsive microgel particles). In some embodiments, a pH responsive microgel particle may be characterized as microgel particles needing to be in the presence of a desired pH range for the annealing of the covalently stabilized scaffold to occur.

In some embodiments, the dermal filler system or the microgel particles comprise glutaraldehyde, divinyl sulfone, 1,4-butanediol diglycidyl ether (BDDE), or a derivative of any of these, or any combination thereof. In some embodiments, one or more of these are configured to interact in a crosslinking reaction to synthesize the microgel particles.

Functional groups disclosed herein may comprise a peptide. Functional groups disclosed herein may comprise an amino acid. In some embodiments, a functional group comprises a K peptide. In some embodiments, the K peptides comprises an amino acid sequence comprising FKGGERCG (SEQ ID NO: 4). In some embodiments, the K peptide comprises an amino acid sequence that is about 75% identical to an amino acid sequence provided in SEQ ID NO: 4. In some embodiments, the K peptide comprises an amino acid sequence that is about 85% identical to an amino acid sequence provided in SEQ ID NO: 4. In some embodiments, the K peptide comprises an amino acid sequence that is about 95% identical to an amino acid sequence provided in SEQ ID NO: 4. In some embodiments, the K peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 4. In some embodiments, the functional group comprises a Q peptide. In some embodiments, the Q peptide comprises an amino acid sequence provided in NQEQVSPLGGERCG (SEQ ID NO: 5). In some embodiments, the Q peptide comprises an amino acid sequence that is about 75% identical to an amino acid sequence provided in SEQ ID NO: 5. In some embodiments, the Q peptide comprises an amino acid sequence that is about 85% identical to an amino acid sequence provided in SEQ ID NO: 5. In some embodiments, the Q peptide comprises an amino acid sequence that is about 95% identical to an amino acid sequence provided in SEQ ID NO: 5. In some embodiments, the Q peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 5.

Functional groups may comprise a non-peptide polymer. Degradable functional groups may also be random sequences, Omi target sequences, Heat-Shock Protein target sequences. The functional group may comprise an amino acid having D chirality. The functional group may comprise an amino acid having L chirality. Functional groups may comprise hydrolytically degradable natural and synthetic polymers consisting of heparin, alginate, poly(ethylene glycol), polyacrylamides, polymethacrylates, copolymers and terpolymers of polycondensates, such as polyesters, polyamides, and other polymers, such as polyurethanes). The functional group may be synthetically manufactured or naturally isolated. The functional group may comprise DNA oligonucleotides with sequences corresponding to: restriction enzyme recognition sequences, CpG motifs, Zinc finger motifs, CRISPR or Cas-9 sequences, Talon recognition sequences, or transcription factor-binding domains. The functional group may be activated on at least two ends by a reactive group, defined as a chemical group allowing the crosslinker to participate in the crosslinking reaction to form the microgel particles (intra-crosslinking within particles) or to anneal particles together to form the covalently stabilized scaffold (inter-crosslinking between particles), where these functionalities can include: cysteine amino acids, synthetic and naturally occurring thiol-containing molecules, carbene-containing groups, vinyl-containing groups, activated esters, acrylates, norborenes, primary amines, hydrazides, phosphenes, azides, epoxy-containing groups, SANPAH containing groups, and diazirine containing groups. In some embodiments, microgel particles themselves may act as crosslinkers. In some embodiments, the functional groups may be degradable.

In some embodiments, the microgel particles may be functionalized with an acrylate, methacrylate, methacrylamide, maleimide, norbornenes, or any other vinyl derivative. For example, the dermal filler system may further comprise two or more acrylates, methacrylates, acrylamides, maleimides, norbomenes, or any combination thereof.

In some embodiments, the microgel particles are drug eluting, such that a therapeutic agent disclosed herein is released by the microgel particles in situ. In some embodiments, the therapeutic agent comprises a pain medication, a local anesthetic, an anti-inflammatory medication, an anti-fibrotic medication, an antibiotic, or an anti-cancer therapeutic agent. In some embodiments, the local anesthetic is ester based. In some embodiments, the ester based local anesthetic comprises benzocaine, chloroprocaine, procaine, proparacaine, tetracaine, amylocaine, or oxybuprocaine, or any combination thereof. In some embodiments, the local anesthetic is amide based. In some embodiments, the amide based local anesthetic comprises articaine, bupivacaine, dibucaine, etidocaine, levobupivacaine, lidocaine, mepivacaine, prilocaine, ropivacaine, sameridine, tonicaine, or cinchocaine, or any combination thereof. In some embodiments, the local anesthetic is or comprises lidocaine. In some embodiments, the local anesthetic consists of lidocaine. In some embodiments, the pain medication comprises codeine, fentanyl, hydrocodone, hydromorphone, meperidine, morphine, oxycodone, or tramadol, or any combination thereof. In some embodiments, the anti-inflammatory medication is a non-steroidal anti-inflammatory drug (NSAID) or a steroid. In some embodiments, the NSAID comprises ibuprofen or naproxen. In some embodiments, the steroid comprises a corticosteroid. In some embodiments, the antibiotic comprises dicloxacillin, erythromycin, or tetracycline. In some embodiments, the anti-fibrotic medication comprises pentoxifylline. In some embodiments, the therapeutic agent comprises an anti-cancer therapeutic agent. In some embodiments, the anti-cancer therapeutic agent comprises alkylating agents, nitrosoureas, anti-metabolites, plant alkaloids (e.g., agents made from natural products), anti-tumor antibiotics, hormonal agents, biological response modifiers, or a combination thereof. In some embodiments, the anti-cancer therapeutic agent is an agent designed to treat skin cancer. In some embodiments, the skin cancer comprises basal cell carcinoma, cutaneous squamous cell carcinoma, melanoma, and merkel cell carcinoma. In some embodiments, the anti-cancer therapeutic agent comprises cisplatin, 5-fluorouracil (5-FU), Aldara (Imiquimod), Cemiplimab-rwlc, Efudex (Fluorouracil—Topical), Erivedge (Vismodegib), 5-FU (Fluorouracil—Topical), Fluorouracil—Topical, Imiquimod, Libtayo (Cemiplimab-rwlc), Odomzo (Sonidegib), Sonidegib, Vismodegib, Cemiplimab-rwlc, Keytruda (Pembrolizumab), Libtayo, Pembrolizumab, Aldesleukin, Binimetinib, Braftovi (Encorafenib), Cobimetinib, Cotellic (Cobimetinib), Dabrafenib, Dacarbazine, DTIC-Dome (Dacarbazine), Encorafenib, IL-2 (Aldesleukin), Imlygic (Talimogene Laherparepvec), Interleukin-2 (Aldesleukin), Intron A (Recombinant Interferon Alfa-2b), Ipilimumab, Keytruda (Pembrolizumab), Kimmtrak (Tebentafusp-tebn), Mekinist (Trametinib Dimethyl Sulfoxide), Mektovi (Binimetinib), Nivolumab, Nivolumab and Relatlimab-rmbw, Opdivo (Nivolumab), Opdualag (Nivolumab and Relatlimab-rmbw), Peginterferon Alfa-2b, Pembrolizumab, Proleukin (Aldesleukin), Recombinant Interferon Alfa-2b, Sylatron (Peginterferon Alfa-2b), Tafinlar (Dabrafenib), Talimogene Laherparepvec, Tebentafusp-tebn, Trametinib Dimethyl Sulfoxide, Vemurafenib, Yervoy (Ipilimumab), Zelboraf (Vemurafenib), Avelumab, Bavencio (Avelumab), or combinations thereof.

In some embodiments, the skin cancer is basal cell carcinoma. In some embodiments, the anti-cancer therapeutic agent comprises Aldara (Imiquimod), Cemiplimab-rwlc, Efudex (Fluorouracil—Topical), Erivedge (Vismodegib), 5-FU (Fluorouracil—Topical), Fluorouracil—Topical, Imiquimod, Libtayo (Cemiplimab-rwlc), Odomzo (Sonidegib), Sonidegib, Vismodegib, or combinations thereof. In some embodiments, the skin cancer is cutaneous squamous cell carcinoma, and the anti-cancer therapeutic agent comprises Cemiplimab-rwlc, Keytruda (Pembrolizumab), Libtayo, Pembrolizumab, or combinations thereof. In some embodiments, the skin cancer is melanoma and the anti-cancer therapeutic agent comprises Aldesleukin, Binimetinib, Braftovi (Encorafenib), Cobimetinib, Cotellic (Cobimetinib), Dabrafenib, Dacarbazine, DTIC-Dome (Dacarbazine), Encorafenib, IL-2 (Aldesleukin), Imlygic (Talimogene Laherparepvec), Interleukin-2 (Aldesleukin), Intron A (Recombinant Interferon Alfa-2b), Ipilimumab, Keytruda (Pembrolizumab), Kimmtrak (Tebentafusp-tebn), Mekinist (Trametinib Dimethyl Sulfoxide), Mektovi (Binimetinib), Nivolumab, Nivolumab and Relatlimab-rmbw, Opdivo (Nivolumab), Opdualag (Nivolumab and Relatlimab-rmbw), Peginterferon Alfa-2b, Pembrolizumab, Proleukin (Aldesleukin), Recombinant Interferon Alfa-2b, Sylatron (Peginterferon Alfa-2b), Tafinlar (Dabrafenib), Talimogene Laherparepvec, Tebentafusp-tebn, Trametinib Dimethyl Sulfoxide, Vemurafenib, Yervoy (Ipilimumab), Zelboraf (Vemurafenib), or combinations thereof. In some embodiments, the skin cancer is merkel cell carcinoma and the anti-cancer therapeutic agent comprises Avelumab, Bavencio (Avelumab), Keytruda (Pembrolizumab), Pembrolizumab, or combinations thereof.

In some embodiments herein, the microgel particles are characterized has having a degree of polydispersity. In some embodiments, the polydispersity of the microgel particles is an indicator as to the heterogeneity of the microgel particles based on molecular weight. In some embodiments, the polydispersity of the microgel particles is an indicator as to the heterogeneity of the microgel particles based on size. In some embodiments, the microgel particles comprise a polydispersity of no more than 0.1 when the polydispersity (PDI) is calculated based on a standard deviation (SD) and mean size of the microgel particles (e.g., PDI=(SD/mean)\hat{ }2). In some embodiments, the microgel particles comprise a polydispersity of no more than 0.1 when the polydispersity (PDI) is calculated based on a weight average (MW) and a number average molecular weight (Mn) of the microgel particles (e.g. PDI=Mw/Mn). In some embodiments, the polydispersity is measured using a coefficient of variant (CV), wherein the coefficient of variation is calculated based on a standard deviation (SD) and mean size of the microgel particles (e.g. CV=SD/mean). In some embodiments, a lower polydispersity, based on microgel particle size or weight, assists in forming the covalently stabilized scaffold. In some embodiments, a lower polydispersity, based on microgel particle size or weight, assists in achieving desired mechanical properties of the covalently stabilized scaffold. In some embodiments, a low polydispersity improves the porosity of the composition (e.g., as the size of particles becomes more polydisperse, this may lead to smaller particles inserting into the pores of the covalently stabilized scaffold).

In some embodiments, the microgel particles comprise a degree of substitution per monomer of about 5% to about 20%. In some embodiments, the microgel particles comprise a degree of substitution per monomer of greater than or equal to about 5%, 10%, 15%, or 20%. In some embodiments, the microgel particles comprise a degree of substitution per monomer that is less than or equal to about 5%, 10%, 15%, or 20%. In some embodiments, the microgel particles comprises modified HA having a degree of substitution per monomer of HA that is about 5% to about 20%. In some embodiments, the microgel particles comprises modified HA having a degree of substitution per monomer of HA that is about 10% to about 20%. In some embodiments, the microgel particles comprises modified HA having a degree of substitution per monomer of HA that is about 12%. In some embodiments, the microgel particles comprises modified PEG having a degree of substitution per monomer of HA that is about 80% to about 100%.

In some embodiments, the components of the dermal filler system discussed above can aid in the synthesis of the microgel particles. In some embodiments, the thiol or the derivative thereof and the VS or the derivative thereof are configured to interact with each other in a reaction to synthesize the microgel particles.

In some embodiments, the reaction comprises a covalent synthesizing reaction. Non-limiting examples of a covalent bond are bonds found in a carbon-carbon, amide, ester, thioether bond, carbamate, disulfide bond, oxime, thiourea, hydrazone, and imine. In some embodiments, the reaction comprises a non-covalent synthesizing reaction. Non-limiting examples of non-covalent bonds are those found in an interaction such as, electrostatic interactions, hydrogen bonding, cation-π, π-π stacking, metal-ligand binding, and van der Waals interactions. In some embodiments, the methods comprise linking two or more microgel particles together. Non-limiting examples of linking reactions include Michael addition, amide bond coupling, “click” chemistry (e.g., Diels-Alder cycloaddition, Huisgen 1,3-dipolar cycloaddition), reductive amination, carbamate linkage, ester linkage, thioether linkage, disulfide bonding, hydrazone bonding, oxime coupling, and thiourea coupling.

In some embodiments, the reaction comprises a covalent synthesizing reaction. In some embodiments, the covalent synthesizing reaction comprises a Michael addition (e.g., thiol-ene Michael addition, aza-Michael addition, oxa-Michael addition) or a pseudo-Michael addition reaction. In some embodiments, the thiol or the derivative thereof is a Michael donor in the Michael addition or pseudo-Michael addition reaction. In some embodiments, the VS or derivative thereof is a Michael acceptor in the Michael addition or pseudo-Michael addition reaction. In some embodiments, the thiol or the derivative thereof and the VS or the derivative thereof are present in the dermal filler system at a molar ratio of about 1:1. In some embodiments, the thiol or the derivative thereof and the VS or the derivative thereof are present in the dermal filler system at a molar ratio of about 0.3:1 thiol:VS to 1:1 thiol:VS. In some embodiments, the thiol or the derivative thereof and the VS or the derivative thereof are present in the dermal filler system at a molar ratio of about 0.6:1 thiol:VS to 0.8:1 thiol:VS. In some embodiments, the thiol or the derivative thereof and the VS or the derivative thereof are present in the dermal filler system at a molar ratio of about 1:1 to about 1:2 thiol:VS. In some embodiments, the thiol or the derivative thereof and the VS or the derivative thereof are present in the dermal filler system at a molar ratio of about 1:1 to about 1:1.4 thiol:VS. In some embodiments, the molar ratio may be defined as the molar ratio of thiol (SH) to vinyl sulfone (VS) groups. R=[SH]/[VS]=nSH/nVS where [SH] is defined as the molar concentration of thiols, [VS] is defined as molar concentration of VS, nSH is defined as molar number of thiols and nVS is defined as molar number of VS. In some embodiments, there is an excess of either of the thiol or the derivative thereof and the VS or the derivative thereof in the dermal filler system such that the excess of either of the thiol or the derivative thereof or the VS or the derivative thereof participates in the annealing reaction to form the porous covalently stabilized scaffold.

In some embodiments, the synthesis of the microgel particles may be accomplished via one or more physical linking points (e.g., looping) of the polymer(s) (e.g., PEG and/or HA) that make up microgel particles. In some embodiments, physical linking may include weak physical interactions. In some embodiments, the weak physical interactions may include coordination bonding and ionic interactions. In some embodiments, the physical linking points assist the reaction in synthesizing the microgel particles. In some embodiments, microgel particle synthesis is accomplished by the reaction alone. In some embodiments, microgel particle synthesis is accomplished by physical linking alone.

### Covalently Stabilized Scaffold

Disclosed herein, in some embodiments, are microgel particles that anneal together to form a covalently stabilized scaffold. In some embodiments, the formation of the covalently stabilized scaffold is performed in situ following delivery of the microgel particles to the tissue site. In some embodiments, the dermal filler system comprises one or more components configured to assist in the annealing reaction to form the porous covalently stabilized scaffold.

In some embodiments, the one or more components that facilitate or induce annealing of the microgel particles in the dermal filler system to form the covalently stabilized scaffold comprises annealing components, annealing agents, or a combination thereof. In some embodiments, the annealing agent comprises a molecule. In some embodiments, the annealing agent comprises a photoinitiator. By way of non-limiting example, the photoinitiator may be Eosin Y. In some embodiments, the annealing agent comprises triethanolamine. In some embodiments, the annealing agent comprises an enzyme. In some embodiments, the enzyme comprises thrombin. In some embodiments, the annealing agent comprises a transglutaminase enzyme. A non-limiting example of a transglutaminase enzyme Factor XIII (Factor XIIIa in its active form). In some embodiments, the annealing agent comprises a radical initiator. In some embodiments, the annealing agent comprises an electron transfer agent. Examples of additional and alternative annealing agents include, by way of non-limiting example, active esters and nucleophiles, catechols that crosslink upon oxidation, and other redox sensitive molecules. In some embodiments, the annealing agents comprise homo or heterofunctional polymers containing thiols, maleimides, vinyl sulfones, methacrylates, methacrylamides, or other vinyl functionalities. In some embodiments, the annealing agent comprises cyclodextrin, cucurbituril, or calixarenes. In some embodiments, the annealing components comprise a K peptide, a Q peptide, or a combination thereof. In some embodiments, the annealing components comprise a vinyl group (e.g. vinyl sulfone, methylacrylate, acrylamide), a thiol, a maleimide, or an amine.

In some embodiments, the microgel particles do not require an annealing agent for annealing. For example, the microgel particles may comprise other components (e.g., functional groups) that participate in a chemical crosslinking reaction to form the covalently stabilized scaffold. In some embodiments, one or more components that facilitate annealing of the microgel particles in the dermal filler system to form the covalently stabilized scaffold comprises a thiol derivative, a vinyl derivative, or a combination thereof. For example, microgel particles may be comprised of a polymer or copolymer that is modified to contain one or more vinyl derivatives and one or more thiol derivatives, in which either of the vinyl derivative and the thiol derivative is in excess. Such vinyl derivative and thiol derivative may undergo a chemical crosslinking reaction to anneal the microgel particles together to form the covalently stabilized scaffold.

In some embodiments, the annealing reaction to anneal the microgel particles together to form the covalently stabilized scaffold comprises a covalent synthesizing reaction. Non-limiting examples of a covalent bond are bonds found in a carbon-carbon, amide, ester, thioether bond, carbamate, disulfide bond, oxime, thiourea, hydrazone, and imine. In some embodiments, the covalent synthesizing reaction comprises a Michael addition (e.g., thiol-ene Michael addition) or a pseudo-Michael addition reaction. In some embodiments, the thiol derivative is a Michael donor in the Michael addition or pseudo-Michael addition reaction. In some embodiments, the vinyl derivative (e.g., vinyl sulfone) is a Michael acceptor in the Michael addition or pseudo-Michael addition reaction. In some embodiments, the covalent synthesizing reaction comprises an ether (oxo-Michael addition) reaction or an amine (aza-Michael addition) reaction.

In some embodiments, the functional groups and the one or more components assisting in the annealing reaction are configured to interact to perform the annealing reaction. In some embodiments, the PEG-dithiol is configured to interact with the excess VS or derivative thereof in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the thiol or derivative thereof of the PEG-dithiol is a Michael donor in the Michael addition or pseudo-Michael addition reaction. In some embodiments, the excess VS is a Michael acceptor in the Michael addition or pseudo-Michael addition reaction.

In some embodiments, the dermal filler system further comprises PEG-divinyl sulfone or a derivative thereof. In some embodiments, the PEG-divinyl sulfone may be the components assisting in the annealing reaction. In some embodiments, the PEG-divinyl sulfone or derivative thereof is configured to interact with the excess thiol or derivative thereof in the annealing reaction to form the porous covalently stabilized scaffold. In some embodiments, the divinyl sulfone or derivative thereof of the PEG-divinyl sulfone is a Michael acceptor in the Michael addition or pseudo-Michael addition reaction. In some embodiments, the excess thiol is a Michael donor in the Michael addition or pseudo-Michael addition reaction.

In some embodiments, the annealing reaction to anneal the microgel particles together to form a stabilized scaffold comprises a non-covalent synthesizing reaction. Non-limiting examples of non-covalent bonds are those found in an interaction such as, electrostatic interactions, hydrogen bonding, cation-π, π-π stacking, metal-ligand binding, and van der Waals interactions. Non-limiting examples of annealing reactions include Michael additions, amide bond coupling, “click” chemistry (e.g. Diels-Alder cycloaddition, Huisgen 1,3-dipolar cycloaddition), reductive amination, carbamate linkage, ester linkage, thioether linkage, disulfide bonding, hydrazone bonding, oxime coupling, and thiourea coupling.

In some embodiments, the molecule comprises PEG. In some embodiments, the molecule comprises PEG-dithiol. In some embodiments, the PEG-dithiol comprises a molecular weight of about 1.0 kDa to about 5.0 kDa. In some embodiments, the PEG-dithiol comprises a molecular weight of about 1, 2, 3, 4, 5, 6, 7, 8, 9, 10, 11, 12, 13, 14, 15, 16, 17, 18, 19, or 20 kDa. In some embodiments, the PEG-dithiol comprises a molecular weight of greater than about 1, 2, 3, 4, 5, 6, 7, 8, 9, 10, 11, 12, 13, 14, 15, 16, 17, 18, 19, or 20 kDa. In some embodiments, the PEG-dithiol comprises a molecular weight of less than about 1, 2, 3, 4, 5, 6, 7, 8, 9, 10, 11, 12, 13, 14, 15, 16, 17, 18, 19, or 20 kDa. In some embodiments, the PEG-dithiol comprises a molecular weight of about 1.0 kDa to about 10.0 kDa. In some embodiments, the PEG-dithiol comprises a molecular weight of about 1.0 kDa to about 15.0 kDa. In some embodiments, the PEG-dithiol comprises a molecular weight of about 1.0 kDa to about 20.0 kDa. In some embodiments, the PEG-dithiol comprises a molecular weight of about 3.0 kDa to about 10.0 kDa. In some embodiments, the PEG-dithiol comprises a molecular weight of about 3.0 kDa to about 5.0 kDa. In some embodiments, the PEG-dithiol comprises a molecular weight of about 3.4 kDa. In some embodiments, the PEG-dithiol comprises linear PEG-dithiol, multi-arm PEG-dithiol, or a combination thereof. In some embodiments, the PEG-thiol comprises a multi-arm PEG-thiol. In some embodiments, the multi-arm PEG-thiol comprises, 4-arm or 6-arm or 8-arm PEG-thiol. In some embodiments, the multi-arm PEG-thiol comprises star-shaped polymer, brushed polymer, branched polymer, comb polymer or dendritic polymer PEG-thiol.

In some embodiments, the microgel particles are annealed to form the covalently stabilized scaffold in the presence of one or more buffers. Non-limiting examples of buffers include 4-(2-hydroxyethyl)-1-piperazineethanesulfonic acid (HEPES) buffer (pH 7.4), calcium chloride (CaCl₂)), phosphate buffered saline (PBS) (pH 6.0 to 8.0), TRIS, piperazine-N,N′-bis(2-ethanesulfonic acid) (PIPES), 2-(N-morpholino)ethanesulfonic acid buffer (MES), or a combination thereof. In some embodiments, an additional stimulus is added to catalyze the reaction, such as light or pH.

In some embodiments, the covalently stabilized scaffold is porous. In some embodiments, the covalently stabilized scaffold comprises pores having a median pore diameter comprising more than or equal to about 5 μm. In some embodiments, the pores comprise a median pore diameter of about 10 μm to about 35 μm. In some embodiments, the pores comprise a median pore diameter of greater than or equal to about 5, 10, 15, 20, 25, 30, 35, 40, 45, 50, 55, 60, 65, or 70 μm. In some embodiments, the pores comprise a median pore diameter of less than or equal to about 5, 10, 15, 20, 25, 30, 35, 40, 45, 50, 55, 60, 65, or 70 μm. In some embodiments, the pores comprise a median pore diameter of about 5 μm to about 70 μm. In some embodiments, the pores comprise a median pore diameter of about 10 μm to about 65 μm. In some embodiments, the pores comprise a median pore diameter of about 15 μm to about 60 μm. In some embodiments, the pores comprise a median pore diameter of about 20 μm to about 55 μm. In some embodiments, the pores comprise a median pore diameter of about 25 μm to about 50 μm. In some embodiments, the pores comprise a median pore diameter of about 30 μm to about 45 μm. In some embodiments, the pores comprise a median pore diameter of about 35 μm to about 40 μm. Median pore diameter may be measured by a process comprising, for a sampling of pores, (1) measuring the area of pore, (2) solving for the radius of the pore using the equation for the area of a circle (i.e., A=pi*r\hat{ }2) and (3) solving for the diameter by multiplying the radius by 2 (i.e., D=2*r).

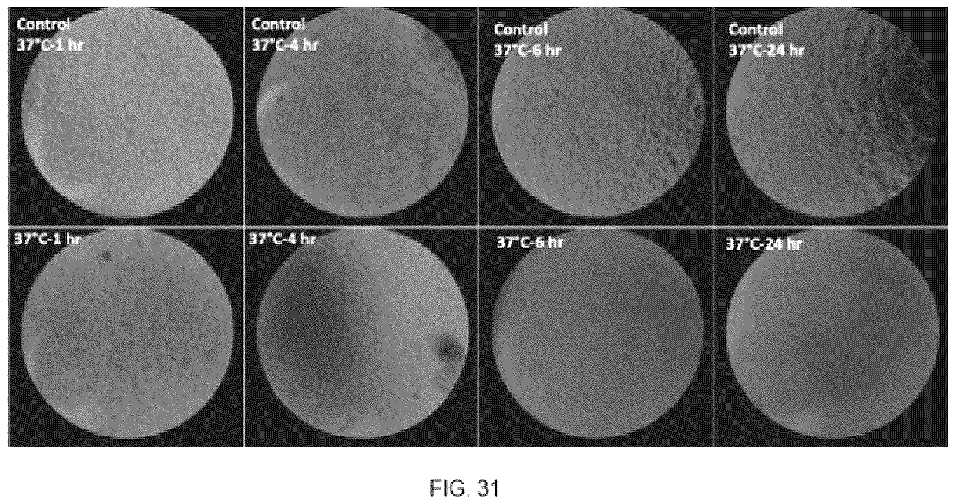

In some embodiments, the covalently stabilized scaffold is degradable in vivo by one or more degradation pathways. In some embodiments, the one or more degradation pathways comprises oxidative degradation, enzymatic degradation, photodegradation, or hydrolytic degradation. In some embodiments, the composition of the microgel particles is fine tuned to achieve a desired degradation profile depending on the application. For example, microgel particles comprising PEG will be degraded slower than a natural polymer, such as HA. Thus, the microgel particles disclosed herein may be a copolymer of PEG and HA to take advantage of the degradation profiles of PEG and HA as well as the other benefits of HA disclosed elsewhere herein. In some embodiments, the dermal filler system is degraded enzymatically by hyaluronidase. In some embodiments, the hyaluronidase is injected to degrade the dermal filler system. In some embodiments, the dermal filler system degrades with endogenous hyaluronidase. In some embodiments, the hyaluronidase degrades the dermal filler system by degrading the HA of the dermal filler system. In some embodiments, it may be desired that an injection of the dermal filler system is reversible. The dermal filler systems described herein, and as shown in FIG. 31, may be degradable upon contact with hyaluronidase after a certain time period has passed. In some embodiments, the dermal filler system is completely degraded after 6 hours using hyaluronidase and at a temperature of 37° C.

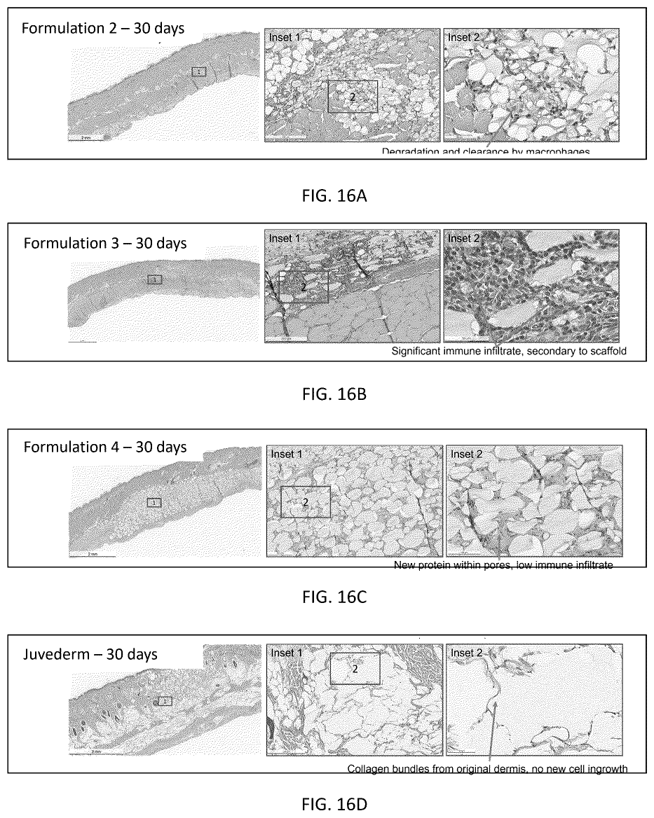

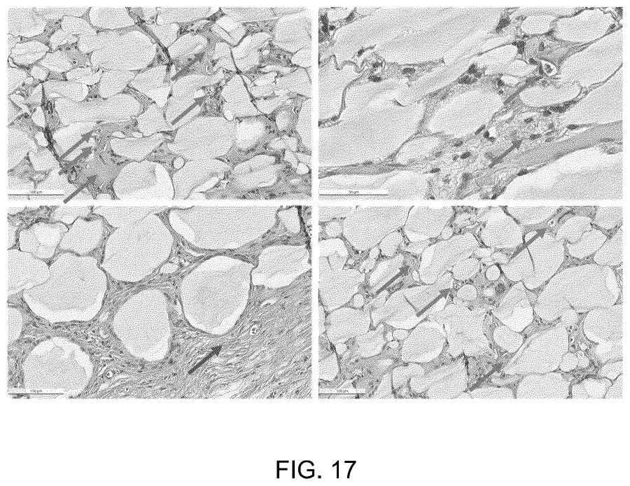

In some embodiments, the covalently stabilized scaffold is present in the tissue site for longer than 9 months before complete degradation. In some embodiments, the covalently stabilized scaffold is present in the tissue site for at least 18 months before complete degradation. In some embodiments, the covalently stabilized scaffold is present in the tissue site for at least 24 months before complete degradation. In some embodiments, the covalently stabilized scaffold is present in the tissue site for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months before complete degradation. As illustrated in FIGS. 16A-16D and FIG. 17, and discussed in Example 2 below, dermal filler systems comprising HA and PEG comprise slower degradation rates than comparable fillers (e.g., JUVÉDERM®) and also allow for increased protein, cell, and tissue growth in the cell matrix. Specifically, FIG. 17 illustrates that dermal filler systems described herein allow for perfused blood vessels within the pore space, new protein deposition within the pore space (e.g., fibrillar, non-aligned), and minimize a foreign body response, such as minimizing or completely preventing the formation of multinucleate giant cells (MNGCs).

In some embodiments, the covalently stabilized scaffold comprises an elastic compressive modulus of 1,000 Pascals (Pa) to 100,000 Pa. In some embodiments, the covalently stabilized scaffold may be annealed when the microgel particles are in a swollen and unswollen state. In some embodiments, the covalently stabilized scaffold may be annealed when the microgel particles are in a swollen state, such as with water. In some embodiments, the microgel particles are in an unswollen state when the covalently stabilized scaffold is annealed. In some embodiments, as discussed above, the elastic compressive modulus of the scaffold can be adjusted by adjusting the molecular weight, percent substitution, and molar ratios of the hydrogel polymer components.

In some embodiments, the covalently stabilized scaffold comprises an elastic compressive modulus of 5,000 Pascals (Pa) to 100,000 Pa in an unswollen state. In some embodiments, the covalently stabilized scaffold comprises an elastic compressive modulus of greater than or equal to about 5,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; 110,000; 115,000; 120,000; 125,000; 130,000; 135,000; 140,000; 145,000; or 150,000 Pa in an unswollen state. In some embodiments, the covalently stabilized scaffold comprises an elastic compressive modulus of less than or equal to about 5,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; 110,000; 115,000; 120,000; 125,000; 130,000; 135,000; 140,000; 145,000; or 150,000 Pa in an unswollen state. In some embodiments, the covalently stabilized scaffold comprises an elastic compressive modulus in a range of about 5,000 Pa to about 150,000 Pa in an unswollen state. In some embodiments, the covalently stabilized scaffold comprises an elastic compressive modulus in a range of about 10,000 Pa to about 145,000 Pa in an unswollen state. In some embodiments, the covalently stabilized scaffold comprises an elastic compressive modulus in a range of about 15,000 Pa to about 140,000 Pa in an unswollen state. In some embodiments, the covalently stabilized scaffold comprises an elastic compressive modulus in a range of about 20,000 Pa to about 135,000 Pa in an unswollen state. In some embodiments, the covalently stabilized scaffold comprises an elastic compressive modulus in a range of about 25,000 Pa to about 130,000 Pa in an unswollen state. In some embodiments, the covalently stabilized scaffold comprises an elastic compressive modulus in a range of about 30,000 Pa to about 125,000 Pa in an unswollen state. In some embodiments, the covalently stabilized scaffold comprises an elastic compressive modulus in a range of about 35,000 Pa to about 120,000 Pa in an unswollen state. In some embodiments, the covalently stabilized scaffold comprises an elastic compressive modulus in a range of about 40,000 Pa to about 115,000 Pa in an unswollen state. In some embodiments, the covalently stabilized scaffold comprises an elastic compressive modulus in a range of about 45,000 Pa to about 110,000 Pa in an unswollen state. In some embodiments, the covalently stabilized scaffold comprises an elastic compressive modulus in a range of about 50,000 Pa to about 105,000 Pa in an unswollen state. In some embodiments, the covalently stabilized scaffold comprises an elastic compressive modulus in a range of about 55,000 Pa to about 100,000 Pa in an unswollen state. In some embodiments, the covalently stabilized scaffold comprises an elastic compressive modulus in a range of about 60,000 Pa to about 95,000 Pa in an unswollen state. In some embodiments, the covalently stabilized scaffold comprises an elastic compressive modulus in a range of about 65,000 Pa to about 90,000 Pa in an unswollen state. In some embodiments, the covalently stabilized scaffold comprises an elastic compressive modulus in a range of about 70,000 Pa to about 85,000 Pa in an unswollen state. In some embodiments, the covalently stabilized scaffold comprises an elastic compressive modulus in a range of about 75,000 Pa to about 80,000 Pa in an unswollen state.

In some embodiments, the covalently stabilized scaffold comprises an elastic compressive modulus of 1,000 Pascals (Pa) to 50,000 Pa in a swollen state. In some embodiments, the covalently stabilized scaffold comprises an elastic compressive modulus of greater than or equal to about 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; or 110,000 Pa in a swollen state. In some embodiments, the covalently stabilized scaffold comprises an elastic compressive modulus of less than or equal to about 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; or 110,000 Pa in a swollen state. In some embodiments, the covalently stabilized scaffold comprises an elastic compressive modulus in a range of about 5,000 Pa to about 110,000 Pa in a swollen state. In some embodiments, the covalently stabilized scaffold comprises an elastic compressive modulus in a range of about 10,000 Pa to about 105,000 Pa in a swollen state. In some embodiments, the covalently stabilized scaffold comprises an elastic compressive modulus in a range of about 15,000 Pa to about 100,000 Pa in a swollen state. In some embodiments, the covalently stabilized scaffold comprises an elastic compressive modulus in a range of about 20,000 Pa to about 95,000 Pa in a swollen state. In some embodiments, the covalently stabilized scaffold comprises an elastic compressive modulus in a range of about 25,000 Pa to about 90,000 Pa in a swollen state. In some embodiments, the covalently stabilized scaffold comprises an elastic compressive modulus in a range of about 30,000 Pa to about 85,000 Pa in a swollen state. In some embodiments, the covalently stabilized scaffold comprises an elastic compressive modulus in a range of about 35,000 Pa to about 80,000 Pa in a swollen state. In some embodiments, the covalently stabilized scaffold comprises an elastic compressive modulus in a range of about 40,000 Pa to about 75,000 Pa in a swollen state. In some embodiments, the covalently stabilized scaffold comprises an elastic compressive modulus in a range of about 45,000 Pa to about 70,000 Pa in a swollen state. In some embodiments, the covalently stabilized scaffold comprises an elastic compressive modulus in a range of about 50,000 Pa to about 65,000 Pa in a swollen state. In some embodiments, the covalently stabilized scaffold comprises an elastic compressive modulus in a range of about 55,000 Pa to about 60,000 Pa in a swollen state.

In some embodiments, a microgel particle suspension in a swollen state is formulated for administration with a needle. In some embodiments, the microgel particle suspension in the swollen state comprises an apparent viscosity of greater than or equal to about 1, 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, or 10000 Pascal-second (Pa*s) when formulated for administration with a needle. In some embodiments, the microgel particle suspension in the swollen state comprises an apparent viscosity of less than or equal to about 1, 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, or 10000 Pa*s when formulated for administration with a needle. In some embodiments, the microgel particle suspension in the swollen state comprises an apparent viscosity of about 1 to about 10000 Pa*s when formulated for administration with a needle. In some embodiments, the microgel particle suspension in the swollen state comprises an apparent viscosity of about 50 to about 9500 Pa*s when formulated for administration with a needle. In some embodiments, the microgel particle suspension in the swollen state comprises an apparent viscosity of about 100 to about 9000 Pa*s when formulated for administration with a needle. In some embodiments, the microgel particle suspension in the swollen state comprises an apparent viscosity of about 150 to about 8500 Pa*s when formulated for administration with a needle. In some embodiments, the microgel particle suspension in the swollen state comprises an apparent viscosity of about 200 to about 8000 Pa*s when formulated for administration with a needle. In some embodiments, the microgel particle suspension in the swollen state comprises an apparent viscosity of about 250 to about 7500 Pa*s when formulated for administration with a needle. In some embodiments, the microgel particle suspension in the swollen state comprises an apparent viscosity of about 300 to about 7000 Pa*s when formulated for administration with a needle. In some embodiments, the microgel particle suspension in the swollen state comprises an apparent viscosity of about 350 to about 6500 Pa*s when formulated for administration with a needle. In some embodiments, the microgel particle suspension in the swollen state comprises an apparent viscosity of about 400 to about 6000 Pa*s when formulated for administration with a needle. In some embodiments, the microgel particle suspension in the swollen state comprises an apparent viscosity of about 450 to about 5500 Pa*s when formulated for administration with a needle. In some embodiments, the microgel particle suspension in the swollen state comprises an apparent viscosity of about 500 to about 5000 Pa*s when formulated for administration with a needle. In some embodiments, the microgel particle suspension in the swollen state comprises an apparent viscosity of about 550 to about 4500 Pa*s when formulated for administration with a needle. In some embodiments, the microgel particle suspension in the swollen state comprises an apparent viscosity of about 600 to about 4000 Pa*s when formulated for administration with a needle. In some embodiments, the microgel particle suspension in the swollen state comprises an apparent viscosity of about 650 to about 3500 Pa*s when formulated for administration with a needle. In some embodiments, the microgel particle suspension in the swollen state comprises an apparent viscosity of about 700 to about 3000 Pa*s when formulated for administration with a needle. In some embodiments, the microgel particle suspension in the swollen state comprises an apparent viscosity of about 750 to about 2500 Pa*s when formulated for administration with a needle. In some embodiments, the microgel particle suspension in the swollen state comprises an apparent viscosity of about 800 to about 2000 Pa*s when formulated for administration with a needle. In some embodiments, the microgel particle suspension in the swollen state comprises an apparent viscosity of about 850 to about 1500 Pa*s when formulated for administration with a needle. In some embodiments, the microgel particle suspension in the swollen state comprises an apparent viscosity of about 900 to about 1000 Pa*s when formulated for administration with a needle. In some embodiments, the microgel particle suspension in the swollen state comprises an apparent viscosity of about 100 to about 1000 Pa*s when formulated for administration with a needle.

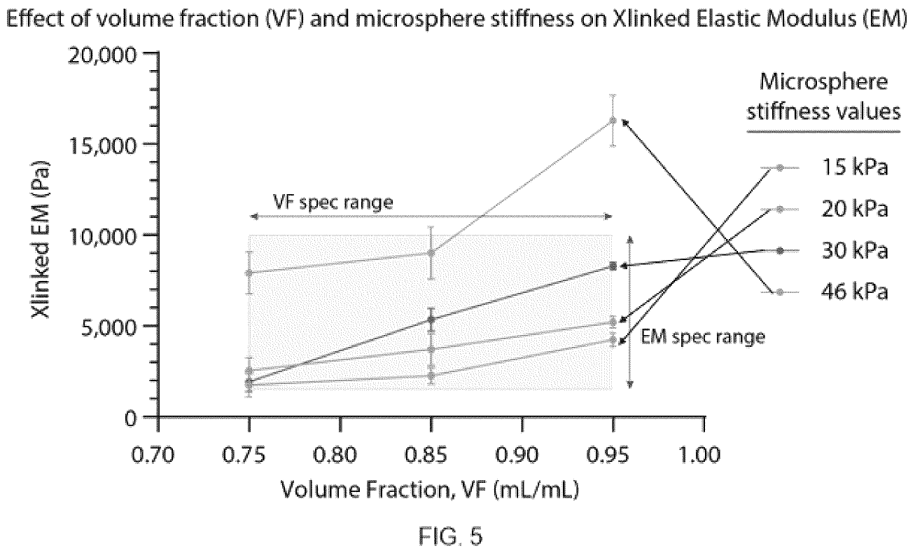

In some embodiments, the volume fraction of the microgel particles and the elastic modulus of the microgel particles can be adjusted to achieve a desired elastic compressive modulus for the covalently stabilized scaffold. As shown in FIG. 5, both volume fraction and elastic modulus of the microgel particles effect the final elastic compressive modulus of the covalently stabilized scaffold. As shown, a higher volume fraction and higher microgel particle elastic modulus may lead to a higher elastic compressive modulus of the covalently stabilized scaffold.

In some embodiments, the covalently stabilized scaffold comprises a storage modulus of 50 Pascals (Pa) to 10,000 Pa in a swollen state. In some embodiments, the covalently stabilized scaffold comprises a storage modulus of 60 Pa to 1,000 Pa in a swollen state. In some embodiments, the covalently stabilized scaffold comprises a storage modulus of greater than or equal to about 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a storage modulus of less than or equal to about 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a storage modulus in a range of about 5,000 Pa to about 110,000 Pa in a swollen state. In some embodiments, the covalently stabilized scaffold comprises a storage modulus in a range of about 10,000 Pa to about 105,000 Pa in a swollen state. In some embodiments, the covalently stabilized scaffold comprises a storage modulus in a range of about 15,000 Pa to about 100,000 Pa in a swollen state. In some embodiments, the covalently stabilized scaffold comprises a storage modulus in a range of about 20,000 Pa to about 95,000 Pa in a swollen state. In some embodiments, the covalently stabilized scaffold comprises a storage modulus in a range of about 25,000 Pa to about 90,000 Pa in a swollen state. In some embodiments, the covalently stabilized scaffold comprises a storage modulus in a range of about 30,000 Pa to about 85,000 Pa in a swollen state. In some embodiments, the covalently stabilized scaffold comprises n storage modulus in a range of about 35,000 Pa to about 80,000 Pa in a swollen state. In some embodiments, the covalently stabilized scaffold comprises a storage modulus in a range of about 40,000 Pa to about 75,000 Pa in a swollen state. In some embodiments, the covalently stabilized scaffold comprises a storage modulus in a range of about 45,000 Pa to about 70,000 Pa in a swollen state. In some embodiments, the covalently stabilized scaffold comprises a storage modulus in a range of about 50,000 Pa to about 65,000 Pa in a swollen state. In some embodiments, the covalently stabilized scaffold comprises a storage modulus in a range of about 55,000 Pa to about 60,000 Pa in a swollen state.

In some embodiments, the microgel particles comprise an elastic compressive modulus of about 5,000 Pa to about 100,000 Pa in an unswollen state. In some embodiments, the microgel particles comprise an elastic compressive modulus of about 5,000 Pa to about 50,000 Pa in an unswollen state. In some embodiments, the microgel particles comprise an elastic compressive modulus of about 5,000 Pa to about 46,000 Pa in an unswollen state. In some embodiments, the microgel particles comprise an elastic compressive modulus of about 5,000 Pa to about 75,000 Pa in an unswollen state. In some embodiments, the microgel particles comprise an elastic compressive modulus of about 5,000 Pa to about 25,000 Pa in an unswollen state. In some embodiments, the microgel particles comprise an elastic compressive modulus of greater than or equal to about 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 Pa or greater in an unswollen state. In some embodiments, the microgel particles comprise an elastic compressive modulus of less than or equal to about 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 Pa or greater in an unswollen state. In some embodiments, the microgel particles comprise an elastic compressive modulus of about 10,000 Pa to about 100,000 Pa in an unswollen state. In some embodiments, the microgel particles comprise an elastic compressive modulus of about 15,000 Pa to about 95,000 Pa in an unswollen state. In some embodiments, the microgel particles comprise an elastic compressive modulus of about 20,000 Pa to about 90,000 Pa in an unswollen state. In some embodiments, the microgel particles comprise an elastic compressive modulus of about 25,000 Pa to about 85,000 Pa in an unswollen state. In some embodiments, the microgel particles comprise an elastic compressive modulus of about 30,000 Pa to about 80,000 Pa in an unswollen state. In some embodiments, the microgel particles comprise an elastic compressive modulus of about 35,000 Pa to about 75,000 Pa in an unswollen state. In some embodiments, the microgel particles comprise an elastic compressive modulus of about 40,000 Pa to about 70,000 Pa in an unswollen state. In some embodiments, the microgel particles comprise an elastic compressive modulus of about 45,000 Pa to about 65,000 Pa in an unswollen state. In some embodiments, the microgel particles comprise an elastic compressive modulus of about 50,000 Pa to about 60,000 Pa in an unswollen state.

In some embodiments, the covalently stabilized scaffold comprises a loss modulus of about 1 Pascals (Pa) to 10,000 Pa in a swollen state. Loss modulus may be measured by undergoing a measurement of shear modulus as described above and performing an amplitude and frequency sweep of shear stress in a parallel plate system. This may enable calculation of both the storage and the loss modulus of the viscoelastic material (together the storage and loss modulus comprise the shear modulus). In some embodiments, the covalently stabilized scaffold comprises a loss modulus that is greater than or equal to about 1, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a loss modulus that is less than or equal to about 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a loss modulus of about 10 Pa to 11,000 Pa, 20 Pa to 10500 Pa, 30 Pa to 10000 Pa, 40 Pa to 9500 Pa, 50 Pa to 9000 Pa, 60 Pa to 8500 Pa, 70 Pa to 8000 Pa, 80 Pa to 7500 Pa, 90 Pa to 7000 Pa, 100 Pa to 6500 Pa, 150 Pa to 6000 Pa, 200 Pa to 5500 Pa, 250 Pa to 5000 Pa, 300 Pa to 4500 Pa, 350 Pa to 4000 Pa, 400 Pa to 3500 Pa, 450 Pa to 3000 Pa, 500 Pa to 2500 Pa, 550 Pa to 2000 Pa, 600 Pa to 1500 Pa, 650 Pa to 1000 Pa, 700 Pa to 950 Pa, 750 Pa to 900 Pa, or 800 Pa to 850 Pa in a swollen state.

In some embodiments, the microgel particles comprise an elastic compressive modulus of from about 500 Pa to about 50,000 Pa in a swollen state. In some embodiments, the microgel particles comprise an elastic compressive modulus of greater than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in a swollen state. In some embodiments, the microgel particles comprise an elastic compressive modulus of less than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in a swollen state. In some embodiments, the microgel particles comprise an elastic compressive modulus of about 500 to 50,000, 1,000 to 45,000, 2,000 to 40,000, 3,000 to 35,000, 4,000 to 30,000, 5,000 to 25,000, 6,000 to 20,000, 7,000 to 20,000, 8,000 to 15,000, or 9,000 to 10,000 Pa in a swollen state. In some embodiments, the microgel particles comprise an elastic compressive modulus of about 500 Pa to 50,000, 500 to 40,000, 500 to 30,000 500 to 20,000, or 500 to 10,000 Pa in a swollen state.

In some embodiments, the microgel particles comprise a storage modulus of from about 10 Pa to about 5,000 Pa in a swollen state. In some embodiments, the microgel particles comprise a storage modulus of greater than about 10, 100, 500, 1,000, 1,500, 2,000, 2,500, 3,000, 3,500, 4,000, 4,500, or 5,000 Pa in a swollen state. In some embodiments, the microgel particles comprise a storage modulus of less than about 10, 100, 500, 1,000, 1,500, 2,000, 2,500, 3,000, 3,500, 4,000, 4,500, or 5,000 Pa in a swollen state. In some embodiments, the microgel particles comprise a storage modulus of about 10 to 5,000, 100 to 4,500, 500 to 4,000, or 1,000 to 3,000 Pa in a swollen state.

In some embodiments, the microgel particles comprise a storage modulus of from about 50 Pa to about 10,000 Pa in an unswollen state. In some embodiments, the microgel particles comprise a storage modulus of greater than about 50, 100, 500, 1,000, 2,000, 3,000, 4,000, 5,000, 6,000, 7,000, 8,000, 9,000, or 10,000 Pa in an unswollen state. In some embodiments, the microgel particles comprise a storage modulus of less than about 50, 100, 500, 1,000, 2,000, 3,000, 4,000, 5,000, 6,000, 7,000, 8,000, 9,000, or 10,000 Pa in an unswollen state. In some embodiments, the microgel particles comprise a storage modulus of about 50 to 10,000, 100 to 9,000, 500 to 8,000, 1,000 to 7,000, 2,000 to 6,000, or 3,000 to 5,000 Pa in an unswollen state.

In some embodiments, the microgel particles comprise a loss modulus of from about 0.1 Pa to about 2,000 Pa in a swollen state. In some embodiments, the microgel particles comprise a loss modulus of greater than about 0.1, 0.5, 1, 50, 100, 500, 1,000, 1,500, or 2,000 Pa in a swollen state. In some embodiments, the microgel particles comprise a loss modulus of less than about 0.1, 0.5, 1, 50, 100, 500, 1,000, 1,500, or 2,000 Pa in a swollen state. In some embodiments, the microgel particles comprise a loss modulus of about 0.1 to 2,000, 0.5 to 1,500, 1 to 1,000, or 50 to 500 Pa in a swollen state.

In some embodiments, the microgel particles comprise a loss modulus of from about 1 Pa to about 5,000 Pa in an unswollen state. In some embodiments, the microgel particles comprise a loss modulus of greater than about 1, 100, 500, 1,000, 1,500, 2,000, 2,500, 3,000, 3,500, 4,000, 4,500, or 5,000 Pa in an unswollen state. In some embodiments, the microgel particles comprise a loss modulus of less than about 1, 100, 500, 1,000, 1,500, 2,000, 2,500, 3,000, 3,500, 4,000, 4,500, or 5,000 Pa in an unswollen state. In some embodiments, the microgel particles comprise a loss modulus of about 1 to 5,000, 100 to 4,500, 500 to 4,000, or 1,000 to 3,000 Pa in an unswollen state.

In some embodiments, a microgel particle suspension comprises an elastic compressive modulus of from about 100 Pa to about 20,000 Pa in a swollen state. In some embodiments, a microgel particle suspension comprises an elastic compressive modulus of greater than about 100, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, or 20,000 Pa in a swollen state. In some embodiments, a microgel particle suspension comprises an elastic compressive modulus of less than about 100, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, or 20,000 Pain a swollen state. In some embodiments, a microgel particle suspension comprises an elastic compressive modulus of about 100 to 20,000, 500 to 19,000, 1,000 to 18,000, 2,000 to 17,000, 3,000 to 16,000, 4,000 to 15,000, 5,000 to 14,000, 6,000 to 13,000, 7,000 to 12,000, 8,000 to 11,000, or 9,000 to 10,000 Pa in a swollen state.

In some embodiments, a microgel particle suspension comprises an elastic compressive modulus of from about 500 Pa to about 50,000 Pa in an unswollen state. In some embodiments, a microgel particle suspension comprises an elastic compressive modulus of greater than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in an unswollen state. In some embodiments, a microgel particle suspension comprises an elastic compressive modulus of less than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in an unswollen state. In some embodiments, a microgel particle suspension comprises an elastic compressive modulus of about 500 to 50,000, 1,000 to 45,000, 2,000 to 40,000, 3,000 to 35,000, 4,000 to 30,000, 5,000 to 25,000, 6,000 to 20,000, 7,000 to 20,000, 8,000 to 15,000, or 9,000 to 10,000 Pa in an unswollen state. In some embodiments, a microgel particle suspension comprises an elastic compressive modulus of about 500 Pa to 50,000, 500 to 40,000, 500 to 30,000 500 to 20,000, or 500 to 10,000 Pa in an unswollen state.

In some embodiments, a microgel particle suspension comprises a storage modulus of from about 10 to about 10,000 Pa in a swollen state. In some embodiments, a microgel particle suspension comprises a storage modulus of greater than about 10, 100, 500, 1,000, 2,000, 3,000, 4,000, 5,000, 6,000, 7,000, 8,000, 9,000 or 10,000 Pa in a swollen state. In some embodiments, a microgel particle suspension comprises a storage modulus of less than about 10, 100, 500, 1,000, 2,000, 3,000, 4,000, 5,000, 6,000, 7,000, 8,000, 9,000 or 10,000 Pa in a swollen state. In some embodiments, a microgel particle suspension comprises a storage modulus of about 10 to 10,000, 100 to 9,000, 500 to 8,000, 1,000 to 7,000, 2,000 to 6,000, or 3,000 to 5,000 Pa in a swollen state.

In some embodiments, a microgel particle suspension comprises a loss modulus of from about 1 to about 10,000 Pa in a swollen state. In some embodiments, a microgel particle suspension comprises a loss modulus of greater than about 1, 100, 500, 1,000, 2,000, 3,000, 4,000, 5,000, 6,000, 7,000, 8,000, 9,000 or 10,000 Pa in a swollen state. In some embodiments, a microgel particle suspension comprises a loss modulus of less than about 1, 100, 500, 1,000, 2,000, 3,000, 4,000, 5,000, 6,000, 7,000, 8,000, 9,000 or 10,000 Pa in a swollen state. In some embodiments, a microgel particle suspension comprises a loss modulus of about 1 to 10,000, 100 to 9,000, 500 to 8,000, 1,000 to 7,000, 2,000 to 6,000, or 3,000 to 5,000 Pa in a swollen state.

In some embodiments, the thiol or the derivative thereof and the vinyl sulfone or the derivative thereof are present in the dermal filler system at a molar ratio of the thiols to the VS (thiol:VS) of about 0.3 to about 0.8 to achieve a desired elastic compressive modulus of about 500 Pa to about 50,000 Pa (e.g., when the dermal filler is formulated for administration with a needle). In some embodiments, the microgel particles are present in a suspension comprising the microgel particles and water and wherein a 50% to 100% volume fraction of the suspension comprises the microgel particles to achieve a desired elastic compressive modulus (e.g., when the dermal filler is formulated for administration with a needle).

In some embodiments, the covalently stabilized scaffold comprises an apparent viscosity of about 1000 to about 1000000 millipascal-second (mPa s) in the shear rate range of 0.1 to 10 s⁻¹. In some embodiments, the volume fraction of the microgel particles and the elastic modulus of the microgel particles can be adjusted to achieve a desired viscosity.

In some embodiments, the covalently stabilized scaffold comprises a pH of 5.0 to 9.0. In some embodiments, the covalently stabilized scaffold comprises a pH of 6.5 to 7.5. In some embodiments, the covalently stabilized scaffold comprises a pH of greater than or equal to about 4.0, 4.5, 5.0, 5.5, 6.0, 6.5, 7.0, 7.5, 8.0, 8.5, 9.0, 9.5, or 10.0. In some embodiments, the covalently stabilized scaffold comprises a pH of less than or equal to about 4.0, 4.5, 5.0, 5.5, 6.0, 6.5, 7.0, 7.5, 8.0, 8.5, 9.0, 9.5, or 10.0. In some embodiments, the covalently stabilized scaffold comprises a pH of about 4.0 to about 10.0. In some embodiments, the covalently stabilized scaffold comprises a pH of about 4.5 to about 9.5. In some embodiments, the covalently stabilized scaffold comprises a pH of about 5.0 to about 9.0. In some embodiments, the covalently stabilized scaffold comprises a pH of about 5.5 to about 8.5. In some embodiments, the covalently stabilized scaffold comprises a pH of about 6.0 to about 8.0. In some embodiments, the covalently stabilized scaffold comprises a pH of about 6.5 to about 7.5. In some embodiments, the covalently stabilized scaffold comprises a pH of about 7.0 to about 7.5.

In some embodiments, the microgel particles comprise a pH of 5.0 to 9.0. In some embodiments, the microgel particles comprise a pH of 6.5 to 7.5. In some embodiments, the microgel particles comprise a pH of greater than or equal to about 4.0, 4.5, 5.0, 5.5, 6.0, 6.5, 7.0, 7.5, 8.0, 8.5, 9.0, 9.5, or 10.0. In some embodiments, the microgel particles comprise a pH of less than or equal to about 4.0, 4.5, 5.0, 5.5, 6.0, 6.5, 7.0, 7.5, 8.0, 8.5, 9.0, 9.5, or 10.0. In some embodiments, the microgel particles comprise a pH of about 4.0 to about 10.0. In some embodiments, the microgel particles comprise a pH of about 4.5 to about 9.5. In some embodiments, the microgel particles comprise a pH of about 5.0 to about 9.0. In some embodiments, the microgel particles comprise a pH of about 5.5 to about 8.5. In some embodiments, the microgel particles comprise a pH of about 6.0 to about 8.0. In some embodiments, the microgel particles comprise a pH of about 6.5 to about 7.5. In some embodiments, the microgel particles comprise a pH of about 7.0 to about 7.5.

In some embodiments, the covalently stabilized scaffold comprises an osmolality of about 100 milliosmole per kilogram (mOsmol/kg) to about 400 mOsmol/kg. In some embodiments, the covalently stabilized scaffold comprises an osmolality of greater than or equal to about 50, 100, 150, 200, 250, 300, 350, 400, 450, or 500 mOsmol/kg. In some embodiments, the covalently stabilized scaffold comprises an osmolality of less than or equal to about 50, 100, 150, 200, 250, 300, 350, 400, 450, or 500 mOsmol/kg. In some embodiments, the covalently stabilized scaffold comprises an osmolality of about 50 mOsmol/kg to about 500 mOsmol/kg. In some embodiments, the covalently stabilized scaffold comprises an osmolality of about 100 mOsmol/kg to about 450 mOsmol/kg. In some embodiments, the covalently stabilized scaffold comprises an osmolality of about 150 mOsmol/kg to about 400 mOsmol/kg. In some embodiments, the covalently stabilized scaffold comprises an osmolality of about 200 mOsmol/kg to about 350 mOsmol/kg. In some embodiments, the covalently stabilized scaffold comprises an osmolality of about 250 mOsmol/kg to about 300 mOsmol/kg.

### Dermal Filler Systems

Disclosed herein, in some embodiments, are dermal filler systems comprising the microgel particles of the present disclosure and an additional active agent (e.g., therapeutic agent), reagent or solvent.

In some embodiments, the dermal filler system comprises microgel particles and a therapeutic agent. In some embodiments, the therapeutic agent is not released from the microgel particles. In some embodiments, the therapeutic agent is released from the microgel particles (e.g., drug-eluting microgel particle). In some embodiments, the active agent is a therapeutic agent. In some embodiments, the therapeutic agent comprises a pain medication, a local anesthetic, an anti-inflammatory medication, an anti-fibrotic medication, or an antibiotic. In some embodiments the local anesthetic is ester based. In some embodiments, the ester based local anesthetic comprises benzocaine, chloroprocaine, procaine, proparacaine, tetracaine, amylocaine, or oxybuprocaine, or any combination thereof. In some embodiments, the local anesthetic is amide based. In some embodiments, the amide based local anesthetic comprises articaine, bupivacaine, dibucaine, etidocaine, levobupivacaine, lidocaine, mepivacaine, prilocaine, ropivacaine, sameridine, tonicaine, or cinchocaine, or any combination thereof. In some embodiments, the local anesthetic is or comprises lidocaine. In some embodiments, the local anesthetic consists of lidocaine. In some embodiments, the lidocaine is present in the dermal filler system at a concentration of about 1.0 mg/mL to about 5.0 mg/mL. In some embodiments, the lidocaine is present in the dermal filler system at a concentration of about 3.0 mg/mL. In some embodiments, the local anesthetic is present in the dermal filler system at a concentration of about 0.5 mg/mL to about 20.0 mg/mL. In some embodiments, the local anesthetic is present in the dermal filler system at a concentration of about 1.0 mg/mL to about 19.5 mg/mL. In some embodiments, the local anesthetic is present in the dermal filler system at a concentration of about 1.5 mg/mL to about 19.0 mg/mL. In some embodiments, the local anesthetic is present in the dermal filler system at a concentration of about 2.0 mg/mL to about 18.5 mg/mL. In some embodiments, the local anesthetic is present in the dermal filler system at a concentration of about 2.5 mg/mL to about 18.0 mg/mL. In some embodiments, the local anesthetic is present in the dermal filler system at a concentration of about 3.0 mg/mL to about 17.5 mg/mL. In some embodiments, the local anesthetic is present in the dermal filler system at a concentration of about 3.5 mg/mL to about 17.0 mg/mL. In some embodiments, the local anesthetic is present in the dermal filler system at a concentration of about 4.0 mg/mL to about 16.5 mg/mL. In some embodiments, the local anesthetic is present in the dermal filler system at a concentration of about 4.5 mg/mL to about 16.0 mg/mL. In some embodiments, the local anesthetic is present in the dermal filler system at a concentration of about 5.0 mg/mL to about 15.5 mg/mL. In some embodiments, the local anesthetic is present in the dermal filler system at a concentration of about 5.5 mg/mL to about 15.0 mg/mL. In some embodiments, the local anesthetic is present in the dermal filler system at a concentration of about 6.0 mg/mL to about 14.5 mg/mL. In some embodiments, the local anesthetic is present in the dermal filler system at a concentration of about 6.5 mg/mL to about 14.0 mg/mL. In some embodiments, the local anesthetic is present in the dermal filler system at a concentration of about 7.0 mg/mL to about 13.5 mg/mL. In some embodiments, the local anesthetic is present in the dermal filler system at a concentration of about 7.5 mg/mL to about 13.0 mg/mL. In some embodiments, the local anesthetic is present in the dermal filler system at a concentration of about 8.0 mg/mL to about 12.5 mg/mL. In some embodiments, the local anesthetic is present in the dermal filler system at a concentration of about 8.5 mg/mL to about 12.0 mg/mL. In some embodiments, the local anesthetic is present in the dermal filler system at a concentration of about 9.0 mg/mL to about 11.5 mg/mL. In some embodiments, the local anesthetic is present in the dermal filler system at a concentration of about 9.5 mg/mL to about 11.0 mg/mL. In some embodiments, the local anesthetic is present in the dermal filler system at a concentration of about 9.5 mg/mL to about 10.5 mg/mL. In some embodiments, the pain medication comprises codeine, fentanyl, hydrocodone, hydromorphone, meperidine, morphine, oxycodone, or tramadol, or any combination thereof. In some embodiments, the anti-inflammatory medication is a non-steroidal anti-inflammatory drug (NSAID) or a steroid. In some embodiments, the NSAID comprises ibuprofen or naproxen. In some embodiments, the steroid comprises a corticosteroid. In some embodiments, the antibiotic comprises dicloxacillin, erythromycin, or tetracycline. In some embodiments, the anti-fibrotic medication comprises pentoxifylline.

In some embodiments, the dermal filler formulation is capable of withstanding sterilization. Sterilization can be carried out by steam sterilization, filtration, microfiltration, e-beam, gamma radiation, ethylene oxide (ETO), light, supercritical carbon dioxide, vaporized hydrogen peroxide or any combination thereof. In some embodiments, certain components of the dermal filler system may be steam sterilized (e.g., autoclaved) without degradation of physical properties, such as the microgel particles. However, when the dermal filler formulation contains an additional component, such as the therapeutic agent, the component of the dermal filler formulation containing the therapeutic agent can be sterilized by means other than heat treatment, such as for example using filtration sterilization.

In some embodiments, sterilization of the dermal filler formulation is by autoclave. Autoclaving can be accomplished by applying a mixture of heat, pressure and moisture to a formulation in need of sterilization. Many different sterilization temperatures, pressures and cycle times can be used. As an example, in some embodiments, the filled syringes may be sterilized at a temperature of at least about 120° C. to about 130° C. or greater. In some embodiments, the filled syringes may be sterilized at a temperature of at least about 120° C. to about 130° C. or greater Moisture may or may not be utilized. In some embodiments, pressure is applied depending on the temperature used in the sterilization process. In some embodiments, the sterilization cycle may be at least about 1 minute to about 20 minutes or more. In some embodiments, the sterilization cycle may be at least about 1 minute to about 30 minutes or more. In some embodiments, the sterilization cycle may be at least about 15 minutes to about 30 minutes or more.

In some embodiments, the method of sterilization incorporates the use of a gaseous species which is known to kill or eliminate transmissible agents. In some embodiments, ethylene oxide is used as the sterilization gas and can sterilize medical devices, products, or any of the delivery devices disclosed herein.

In some embodiments, the method of sterilization incorporates the use of an irradiation source to kill or eliminate transmissible agents. A beam of irradiation is targeted at the delivery device (e.g., syringe) containing the dermal filler formulation, and the wavelength of energy kills or eliminates the unwanted transmissible agents. As a non-limiting example, energy useful includes, but is not limited to ultraviolet (UV) light, electron (e-beam) irradiation, gamma irradiation, visible light, microwaves, or any other wavelength or band of wavelengths which kills or eliminates the unwanted transmissible agents, preferably without substantially altering of degrading the dermal filler formulation.

In some embodiments, the dermal filler system comprises a reagent, such as an annealing agent that facilitates the annealing reaction of the dermal filler system to form a covalently stabilized scaffold. In some embodiments, the annealing agent comprises a photoinitiator. By way of non-limiting example, the photoinitiator may be Eosin Y. In some embodiments, the annealing agent comprises triethanolamine. In some embodiments, the annealing agent comprises an enzyme. In some embodiments, the enzyme comprises thrombin. In some embodiments, the annealing agent comprises a transglutaminase enzyme. A non-limiting example of a transglutaminase enzyme Factor XIII (Factor XIIIa in its active form). In some embodiments, the annealing agent comprises a free radical transfer agent. In some embodiments, the annealing agent comprises an electron transfer agent. Examples of additional and alternative annealing agents include, by way of non-limiting example, include active esters and nucleophiles, catechols that crosslink upon oxidation, and other redox sensitive molecules. In some embodiments, the annealing components comprise a K peptide, a Q peptide, or a combination thereof. In some embodiments, the reagent comprises a stabilization agent, a sterilization agent, or a heat protectant. Non-limiting examples of stabilization agents include reagents, salts, and additives. Non-limiting examples of sterilization agents include reagents, salts, and additives. Non-limiting examples of heat protectants include antioxidants, glycerine, and PEG. In some embodiments, the dermal filler system is protected during sterilization by freezing the dermal filler prior and/or during the terminal sterilization (e.g. irradiation). In some embodiments, the dermal filler system is protected during sterilization by placing the material under a sealed inert atmosphere or under sous-vide ampule.

In some embodiments, the dermal filler system comprises a solvent, such as one or more buffers, water, or a combination thereof. In some embodiments, the microgel particles are present in a suspension comprising water. In some embodiments, the dermal filler system comprises a buffer. In some embodiments, the buffer comprises: a phosphate buffer, a 4-(2-hydroxyethyl)-1-piperazineethanesulfonic acid (HEPES) buffer, a phosphate buffer, or an acetate buffer, a citrate buffer, a borate buffer, or any combination thereof. In some embodiments, the buffer adjusts the pH of the dermal filler system to a desired pH. The pH of the disclosed dermal filler formulations can be about 5.0 to about 8.0, or about 6.5 to about 7.5. In certain embodiments, the pH of the formulation is about 7.0 to about 7.4 or about 7.1 to about 7.3. In some embodiments, the dermal filler system comprises a suspension of the microgel particles in an aqueous solvent, including the buffer. In some embodiments, the buffer may be a buffering agent. In some embodiments, a 50% to 100% volume fraction of the suspension comprises the microgel particles. In some embodiments, at least a 30%, 40%, 50%, 60%, 70%, 80%, 90%, or 100% volume fraction of the suspension comprises the microgel particles. In some embodiments, the volume fraction of the microgel particles is greater than or equal to about 50% when the dermal filler is formulated for administration with a needle. In some embodiments, the volume fraction of the microgel particles is greater than or equal to about 50%, 60%, 70%, 80%, or 90% when the dermal filler is formulated for administration with a needle.

In some embodiments, the dermal filler system is colorless. In some embodiments, the dermal filler system is substantially optically clear. In some embodiments, the polydispersity of the dermal filler system is no more than 0.1. In some embodiments, the coefficient of variance of the dermal filler system is no more than about 62.5%, 60%, 57.5%, 55%, 52.5%, 50%, 47.5%, 45%, 42.5%, 40%, 37.5%, 35%, 32.5%, 30%, 27.5%, 25%, 22.5%, 20%, 17.5%, 15%, 12.5%, or 10%. In some embodiments, the coefficient of variance of the dermal filler system is no more than about 62.5%. In some embodiments, the coefficient of variance of the dermal filler system is no more than about 30%. In some embodiments, the dermal filler system is odorless.

In some embodiments, the dermal filler system is formulated for administration to a subject. In some embodiments, the administration is subdermal administration, dermal administration, intradermal administration, or subcutaneous administration. In some embodiments, administration minimizes a foreign body response in the subject. In some embodiments, the dermal filler system is formulated for administration by a needle. In some embodiments, the dermal filler system is formulated for administration by a microneedle or microneedle patch. In some embodiments, the dermal filler system is formulated for administration by a needle that has a gauge that is about 26-, 27-, 28-, 29-, or 30-gauge. For example, a dermal filler system formulated for a 27-gauge syringe may have an apparent viscosity of about 1000 to 1000000 milli-Pascal*seconds when measured at shear rates between 0.1 and 10 s⁻¹. While, a dermal filler system formulated for a 30-gauge syringe may have an apparent viscosity of about 1000 to 500000 milli-Pascal*seconds when measured at shear rates between 0.1 and 10 s⁻¹. Thus, the dermal filler system properties may be fine-tuned depending on the mode of administration.

In some embodiments, a dose of the dermal filler system comprises a volume of about 0.75 milliliter (mL) to about 1.0 mL. In some embodiments, the volume comprises about 0.5 mL to about 3.0 mL. In some embodiments, the volume comprises 0.75 mL to about 2.75 mL, 1.0 mL to about 2.25 mL, 1.25 mL to about 2.0 mL, 1.0 mL to about 1.75 mL, or 1.25 mL to 1.50 mL. In some embodiments, the dose comprises at greater than or equal to about 0.50 mL, 0.75 mL, 1.0 mL, 1.25 mL, 1.5 mL, 1.75 mL, 2.0 mL, 2.25 mL, 2.5 mL, 2.75 mL, or 3.0 mL. The dose of the dermal filler system may depend on the area of administration. For example, for the dose may be 1.0 mL to 3.0 mL for the midface or malar region, whereas the dose may be 0.50 mL to 2.0 mL for the submalar region. In another example, the dose for the area under the eye may be from 0.50 mL to 1.0 mL. The dose for the chin may be 1.0 mL to 3.0 mL. In some embodiments, the dose for the lips may be 0.50 mL to 3.0 mL. In some embodiments, the area of administration comprises any one of the tissue sites disclosed herein and the dose comprises about 0.50 mL to about 3.0 mL.

In some embodiments, the dermal filler system is aseptically manufactured. In some embodiments, the dermal filler system is sterile. In some embodiments, the dermal filler is formulated for sterilization by steam sterilization, filtration, microfiltration, e-beam, gamma radiation, ethylene oxide (ETO), light, supercritical carbon dioxide, vaporized hydrogen peroxide or any combination thereof. In some embodiments, the dermal filler system comprises at least two separate containers, each container suitable for sterilization by different methods. In some embodiments, the microgel particles are lyophilized. In some embodiments, the lyophilized microgel particles are stored in a first container that is capable of withstanding steam sterilization, which is separate from a second container comprising components of the dermal filler system that may be degradable by steam sterilization, such as the therapeutic agent. In another embodiment, the dermal filler system is stored in a single container capable of being sterilized together. In some embodiments, the system also comprises a reconstitution medium to reconstitute the lyophilized dermal filler system. In some embodiments, the reconstitution medium comprises a physiologically isotonic buffer such as phosphate buffered saline. In some embodiments, the reconstitution medium has a pH higher than the physiological pH. In some embodiments, the reconstitution medium has a pH lower than the physiological pH. In some embodiments, the reconstitution medium comprises a buffer with a varying buffer capacity.

### Delivery Devices

Disclosed herein, in some embodiments, are delivery devices configured to deliver the dermal filler system to a subject. A non-limiting example of a delivery device is a needle, or a microneedle (e.g., microneedle patch). In some embodiments, the delivery device comprises a body and an applicator in fluidic communication with the body. In some embodiments, the body is elongated (e.g., a barrel). In some embodiments, the body of the delivery device comprises an inner chamber that contains the dermal filler system. In some embodiments, the delivery device comprises a pump or a plunger configured to apply pressure to the dermal filler system contained in the body under conditions that the dermal filler flows through and out of the applicator via an outlet of the applicator. In some embodiments, the body of the delivery device comprises a first chamber for the microgel particles and a second chamber for the annealing agents and/or components. In some embodiments, the delivery device mixes the components of the first chamber and the second chamber. In some embodiments, the syringe is prefilled with the dermal filler system. In some embodiments the syringe is sterile. In some embodiments, the syringe is packaged separately from the dermal filler system and both the syringe and the dermal filler system are sterile.

### Methods

Disclosed herein, in some embodiments, are methods of preparing or using the dermal filler systems disclosed herein. In some embodiments, the methods disclosed herein comprise delivering the dermal filler systems disclosed herein to a subject. In some embodiments, the delivering comprising administering the dermal filler systems to the subject. In some embodiments, administration comprises subdermal, dermal, intradermal, or subcutaneous administration of the dermal filler system to a tissue site of the subject. In some embodiments, administering the dermal filler system to a tissue site of the subject is effective to treat the tissue at or surrounding the tissue site, such as for example, treating a cancer of the tissue or improving an aesthetic quality of tissue (e.g., reducing wrinkles or fine lines, filling the tissue, repairing the tissue, correcting skin irregularities, treating one or more dermatological conditions). In some embodiments, improving an aesthetic quality of tissue comprises filling at least part of the tissue site of the subject. In some embodiments, filling comprises forming new tissue within the cell matrix at the tissue site. In some embodiments, the new tissue has a persistence time in vivo at tissue site similar to endogenous tissue surrounding the tissue site. In some embodiments, methods of delivering or administering the dermal filler system disclosed herein is performed while minimizing a foreign body response elicited by the subject in response to the delivering or the administering. Also provided are methods for purifying the microgel particles of the dermal filler systems disclosed herein, such as for example, in a water-in-oil emulsion. In some embodiment, the methods comprise lyophilizing the microgel particles to that they may be stored and/or distributed over long periods of time prior to being reconstituted and delivered at the point of need to the subject.

### Methods of Delivery

Disclosed herein, in some embodiments, are methods of delivering the dermal filler systems provided herein to a subject. In some embodiments, the dermal filler is delivered to a tissue site of a subject. In some embodiments, the method comprises delivering to the tissue site the dermal filler system under conditions sufficient for the microgel particles to anneal to one another to form a porous covalently stabilized scaffold. In some embodiments, the formation of the porous covalently stabilized scaffold happens in vivo, in situ, or both in vivo and in situ. In some embodiments, the porous covalently stabilized scaffold forms under conditions sufficient to allow cells to grow within the pores of the porous covalently stabilized scaffold to produce a cell matrix. In some embodiments, the cell matrix that forms persists in the subject after complete degradation of the porous covalently stabilized scaffold, thereby filling at least a part of the tissue site of the subject while minimizing a foreign body response in the subject. In some embodiments, filling comprises forming new tissue within the cell matrix at the tissue site. In some embodiments, the new tissue has a persistence time in vivo at tissue site similar to endogenous tissue surrounding the tissue site. The methods of delivery disclosed herein may be subdermal, dermal, intradermal, or subcutaneous. In some embodiments, the methods of delivery comprise injection, such as for example, using a delivery device (e.g., syringe) disclosed herein.

In some embodiments, the delivering comprises performing subdermal administration of the dermal filler system to a subject. In some embodiments, the delivering comprises performing dermal administration of the dermal filler system to a subject. In some embodiments, the delivering comprises performing intradermal administration of the dermal filler system to a subject. In some embodiments, the delivering comprises performing subcutaneous administration of the dermal filler system to a subject. In some embodiments, the delivering comprises releasing the dermal filler formulation from a syringe or needle. In some embodiments, the needle has a gauge comprising about 25 gauge to about 35 gauge. In some embodiments, the needle has a gauge comprising about a 27 gauge. In some embodiments, the needle has a gauge comprising about a 30 gauge. In some embodiments, the needle has a gauge comprising about 20, 21, 22, 23, 24, 25, 26, 27, 28, 29, 30, 31, 32, 33, 34, 35, 36, 37, 38, 39, or 40 gauge. In some embodiments, the delivering comprises exerting an extrusion force of up to 40 Newtons (N) on the dermal filler system. In some embodiments, the delivering comprises exerting an extrusion force of up to 12 Newtons (N) on the dermal filler system. In some embodiments, the delivering comprises exerting an extrusion force of up to 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 N.

In some embodiments, the syringe may have an internal volume of about 0.1 mL to about 3 mL. In some embodiments, the internal volume comprises about 0.5 mL to about 3.0 mL. In some embodiments, the volume comprises 0.75 mL to about 2.75 mL, 1.0 mL to about 2.25 mL, 1.25 mL to about 2.0 mL, 1.0 mL to about 1.75 mL, or 1.25 mL to 1.50 mL. In some embodiments, the syringe comprises a dose comprising greater than or equal to about 0.50 mL, 0.75 mL, 1.0 mL, 1.25 mL, 1.5 mL, 1.75 mL, 2.0 mL, 2.25 mL, 2.5 mL, 2.75 mL, or 3.0 mL. The dose of the dermal filler system may depend on the area of administration. In some embodiments, the internal volume is associated with an internal diameter of the syringe which affects the extrusion force needed to inject the dermal filler compositions. In some embodiments, the internal diameters may be about 4 mm to about 9 mm. In some embodiments, the internal diameters may be about 4.5 mm to about 6.5 mm. In some embodiments, the internal diameters may be about 4.5 mm to about 8.8 mm. In some embodiments, the extrusion force needed to deliver the HA compositions from the syringe is dependent on the needle gauge. In some embodiments, the compositions are packaged in a 1 mL to about 3 mL syringe and injected using a 20 gauge to about 40 gauge needle.

In some embodiments, methods comprise delivering the hydrogel microparticles (e.g., as disclosed herein) and the annealing agent (e.g., as disclosed herein) to the subject separately. In some embodiments, methods comprise delivering the hydrogel microparticles (e.g., as disclosed herein) and the annealing agent (e.g., as disclosed herein) to the subject together. In some embodiments, the hydrogel microparticles (e.g., as disclosed herein) and the annealing agent (e.g., as disclosed herein) have a shelf life of at least about 18 months when the hydrogel microparticles and the annealing agent are stored in a single container as a mixture. In some embodiments, the hydrogel microparticles (e.g., as disclosed herein) and the annealing agent (e.g., as disclosed herein) have a shelf life of at least about 36 months when the hydrogel microparticles and the annealing agent are stored in a single container as a mixture. In some embodiments, the shelf life of the hydrogel microparticles (e.g., as disclosed herein) and the annealing agent (e.g., as disclosed herein) as a mixture corresponds to an amount of time (e.g., 18 or 36 months) when the mixture is stored at room temperature. In some embodiments, the hydrogel microparticles (e.g., as disclosed herein) and the annealing agent (e.g., as disclosed herein) have a shelf life of at least about 18 months when the hydrogel microparticles and the annealing agent are stored in separate containers. In some embodiments, the hydrogel microparticles (e.g., as disclosed herein) and the annealing agent (e.g., as disclosed herein) have a shelf life of at least about 36 months when the hydrogel microparticles and the annealing agent are stored in separate containers. In some embodiments, the shelf life of the hydrogel microparticles (e.g., as disclosed herein) and the annealing agent (e.g., as disclosed herein) when stored in separate containers corresponds to an amount of time (e.g., 18 or 36 months) when stored at room temperature.

Disclosed herein are methods comprising administering the dermal filler systems described herein to a tissue site of a subject. In some embodiments, the tissue site comprises a midface or malar region of the subject, a cheek of the subject, a jawline of the subject, or the lips of the subject, or any combination thereof. In some embodiments, the midface region comprises the facial region including the nose, cheek, lips (e.g., upper lip) and posteriorly extends to the anterior skull base. In some embodiments, in the superoinferior direction, the midface region includes the soft and bony tissue from the orbital cavity to the oral cavity. In some embodiments, laterally, the midface region extends to the temporal bone. In some embodiments, the malar region comprises the most medial and superior part of the maxilla. In some embodiments, it forms the medial border of the inferior bony orbit and is contiguous with the lateral boundary of the nasal bridge. In some embodiments, the cheeks are comprised of the region below the eyes and above the jawline. In some embodiments, the cheeks span between the nose and the ears. In some embodiments, the cheek is comprised of the soft tissues between the zygoma and the mandible. In some embodiments, the tissue site comprises the forehead region. In some embodiments, the forehead region is comprised of the part of the face above the eyebrows, below the hairline and between the temples. In some embodiments, the tissue site comprises the lower face region of the subject. In some embodiments, the lower face is comprised of the area between the mouth and the inferior point of the chin. In some embodiments, the tissue site comprises the chin of the subject. In some embodiments, the chin is comprised of the inferior portion of the face lying inferior to the lower lip and including the central prominence of the lower jaw. In some embodiments, the tissue site comprises skin folds. In some embodiments, the tissue site comprises the nasolabial folds. In some embodiments, the nasolabial folds are comprised of the lines extending from the sides of the nose to the edges of the mouth. In some embodiments, the tissue site comprises the perioral lines of the subject. In some embodiments, the perioral lines are comprised of the small wrinkles in the skin around the mouth and lips. In some embodiments, the tissue site is on the limbs of the subject (e.g., arms, legs, hands, feet, etc.). In some embodiments, the tissue site is one or more digits of the subject (e.g., fingers, toes). In some embodiments, the tissue site comprises a wound site of the subject. In some embodiments, the wound site comprises a site of abrasion, avulsion, incision, laceration, puncture, or a combination thereof of the skin. In some embodiments, the wound site comprises a burn site. In some embodiments, the tissue site comprises a site of scarring (e.g., a site where a mark is left on the skin or within body tissue where a wound, burn, or sore has not healed completely and fibrous connective tissue has developed of the subject). In some embodiments, the scars are keloid, hypertrophic, contracture, adhesion, or a combination thereof. In some embodiments, the scarring is a result of acne. In some embodiments, the tissue site is a surgical site of a subject. In some embodiments, the tissue site is anywhere comprising soft tissue. In some embodiments, the tissue site is anywhere comprising connective tissue. In some embodiments, the tissue site is anywhere comprising epithelial tissue. In some embodiments, the tissue site is anywhere comprising muscle tissue. In some embodiments, the tissue site is anywhere comprising nervous tissue. In some embodiments, methods comprise administering a dose of the dermal filler system to the subject, which may depend on the location and/or tissue at the tissue site as well as the intended therapeutic or aesthetic effect.

In some embodiments, methods disclosed herein comprise delivering the dermal filler system to the subject under conditions sufficient for adjacent microgel particles to anneal to each other to form a stabilized scaffold. In some embodiments, the stabilized scaffold that forms is covalently stabilized (e.g., covalent interactions between the adjacent microgel particles facilitate the annealing). In some embodiments, the stabilized scaffold is porous. In some embodiments, methods comprise performing the annealing reaction of the microgel particles to form a covalently stabilized scaffold. In some embodiments, the covalently stabilized scaffold is any of the covalently stabilized scaffolds described herein. In some embodiments, methods comprise forming the covalently stabilized scaffold in a manner such that pores form between the microgel particles of the covalently stabilized scaffold (e.g., the covalently stabilized scaffold is porous).

In some embodiments, methods disclosed herein comprise delivering the dermal filler system to the subject under conditions sufficient for endogenous cells of the subject to infiltrate and grow within porous covalently stabilized scaffold. In some embodiments, the cells form a cell matrix within the porous covalently stabilized scaffold. In some embodiments, the porous covalently stabilized scaffold persists at the tissue site for a length of time sufficient for the cell matrix to grow into tissue in situ. In some embodiments, the methods comprise vascularizing, depositing extracellular matrix, or producing proteins and enzymes in the tissue site that aid in treating the tissue site, or any combination thereof. In some embodiments, the methods comprise forming new tissue from the cell matrix at the injection or tissue site. In some embodiments, the new tissue is characterized by having mature vascularization, a characteristic of surrounding tissue at the tissue site, or a combination thereof. In some embodiments, the characteristic of the surrounding tissue at the tissue site comprises functionally differentiated cell types from the surrounding tissue. Non-limiting examples of functionally differentiated cell types from epithelial tissue include squamous, cuboidal and columnar cells. Non-limiting examples of functionally differentiated cell types from the dermis include fibroblasts, macrophages, adipocytes, mast cells, Schwann cells, and stem cells. Non-limiting examples of functionally differentiated cell types from the epidermis include keratinocytes, melanocytes, Langerhans cells, and Merkel cells. Non-limiting examples of functionally differentiated cell types from the dermis include fibroblasts, adipose cells, and macrophages. In some embodiments, the dermal filler completely degrades in vivo, while the new tissue formed from the cell matrix persist. In some embodiments, the new tissue is characterized as having an extracellular matrix. In some embodiments, the dermal filler partially degrades in vivo.

In some embodiments, the at least part of the tissue site comprising the cell matrix comprises at least 25% of the tissue site following degradation of the porous covalently stabilized scaffold at the tissue site. In some embodiments, the at least part of the tissue site comprising the cell matrix comprises at least 10%, 15%, 20%, 25%, 30%, 35%, 40%, 45%, 50%, 55%, 60%, 65%, 75%, 80%, 85%, 90%, or 95% or greater of the tissue site following degradation of the porous covalently stabilized scaffold at the tissue site. In some embodiments, the at least part of the tissue site comprising the cell matrix comprises at least about 10% to about 50% of the tissue site following degradation of the covalently stabilized scaffold at the tissue site. In some embodiments, the at least part of the tissue site comprising the cell matrix comprises at least about 15% to about 45% of the tissue site following degradation of the covalently stabilized scaffold at the tissue site. In some embodiments, the at least part of the tissue site comprising the cell matrix comprises at least about 20% to about 40% of the tissue site following degradation of the covalently stabilized scaffold at the tissue site. In some embodiments, the at least part of the tissue site comprising the cell matrix comprises at least about 25% to about 35% of the tissue site following degradation of the covalently stabilized scaffold at the tissue site. In some embodiments, the at least part of the tissue site comprising the cell matrix comprises at least about 10% to about 90% of the tissue site following degradation of the covalently stabilized scaffold at the tissue site. In some embodiments, the at least part of the tissue site comprising the cell matrix comprises at least about 15% to about 90% of the tissue site following degradation of the covalently stabilized scaffold at the tissue site. In some embodiments, the at least part of the tissue site comprising the cell matrix comprises at least about 20% to about 85% of the tissue site following degradation of the covalently stabilized scaffold at the tissue site. In some embodiments, the at least part of the tissue site comprising the cell matrix comprises at least about 25% to about 80% of the tissue site following degradation of the covalently stabilized scaffold at the tissue site. In some embodiments, the at least part of the tissue site comprising the cell matrix comprises at least about 30% to about 75% of the tissue site following degradation of the covalently stabilized scaffold at the tissue site. In some embodiments, the at least part of the tissue site comprising the cell matrix comprises at least about 35% to about 70% of the tissue site following degradation of the covalently stabilized scaffold at the tissue site. In some embodiments, the at least part of the tissue site comprising the cell matrix comprises at least about 40% to about 65% of the tissue site following degradation of the covalently stabilized scaffold at the tissue site. In some embodiments, the at least part of the tissue site comprising the cell matrix comprises at least about 45% to about 60% of the tissue site following degradation of the covalently stabilized scaffold at the tissue site. In some embodiments, the at least part of the tissue site comprising the cell matrix comprises at least about 50% to about 55% of the tissue site following degradation of the covalently stabilized scaffold at the tissue site. In some embodiments, methods comprise growing cells within the porous stabilized scaffold in less than or equal to about one day following the delivering. In some embodiments, methods comprise forming the cell matrix within the porous stabilized scaffold in less than or equal to about 30 days following the delivering. In some embodiments, the cell matrix begins to form within the scaffold within 7 days after administration.

In some embodiments, methods of delivering the dermal filler system disclosed herein minimize a foreign body response elicited by the subject in response to the dermal filler system. In some embodiments, the foreign body response is characterized by chronic inflammation. In some embodiments, the foreign body response is characterized by granuloma formation. In some embodiments, the foreign body response is characterized by scar tissue formation. In some embodiments, the foreign body response is characterized by nodule formation. In some embodiments, the foreign body response is characterized by swelling, pain, or any combination thereof. In some embodiments, the chronic inflammation, granuloma formation, nodule formation, swelling, pain or anything combination is localized to, or around, the tissue site. In some embodiments, the foreign body response is caused at a location other than the tissue site. In some embodiments, the foreign body response is characterized by a presence of multinucleate giant cells (MNGCs) (e.g., fusion of monocytes or macrophages) at the tissue site of the subject. In some embodiments, the foreign body response is characterized by the persistence of MNGCs over an extended period of time. In some embodiments, the period of time comprising greater than or equal to about 1, 2, 3, or 4 weeks or more. In some embodiments, the period of time comprises greater than or equal to about 1, 2, 3, 4, 5, 6, 7, 8, 9, 10, 11, or 12 months or more. In some embodiments, the period of time comprises greater than or equal to about 1, 2, 3, 4, 5, 6, 7, 8, 9, 10, 11, or 12 years or more. In some embodiments, minimizing the foreign body response is characterized as avoiding any formation of MNGCs at the tissue site. In some embodiments, minimizing the foreign body response is characterized as the absence of MNGCs at the tissue site after a period of time after delivering the dermal filler system. In some embodiments, the period of time after delivering the dermal filler system comprises 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 days or less. In some embodiments, the period of time after delivering the dermal filler system comprises 1 to 30 days. In some embodiments, the period of time after delivering the dermal filler system comprises 1 to 29 days. In some embodiments, the period of time after delivering the dermal filler system comprises 1 to 28 days. In some embodiments, the period of time after delivering the dermal filler system comprises 1 to 27 days. In some embodiments, the period of time after delivering the dermal filler system comprises 1 to 26 days. In some embodiments, the period of time after delivering the dermal filler system comprises 1 to 25 days. In some embodiments, the period of time after delivering the dermal filler system comprises 1 to 24 days. In some embodiments, the period of time after delivering the dermal filler system comprises 1 to 23 days. In some embodiments, the period of time after delivering the dermal filler system comprises 1 to 22 days. In some embodiments, the period of time after delivering the dermal filler system comprises 1 to 21 days. In some embodiments, the period of time after delivering the dermal filler system comprises 1 to 20 days. In some embodiments, the period of time after delivering the dermal filler system comprises 1 to 19 days. In some embodiments, the period of time after delivering the dermal filler system comprises 1 to 18 days. In some embodiments, the period of time after delivering the dermal filler system comprises 1 to 17 days. In some embodiments, the period of time after delivering the dermal filler system comprises 1 to 16 days. In some embodiments, the period of time after delivering the dermal filler system comprises 1 to 15 days. In some embodiments, the period of time after delivering the dermal filler system comprises 1 to 14 days. In some embodiments, the period of time after delivering the dermal filler system comprises 1 to 13 days. In some embodiments, the period of time after delivering the dermal filler system comprises 1 to 12 days. In some embodiments, the period of time after delivering the dermal filler system comprises 1 to 11 days. In some embodiments, the period of time after delivering the dermal filler system comprises 1 to 10 days. In some embodiments, the period of time after delivering the dermal filler system comprises 1 to 9 days. In some embodiments, the period of time after delivering the dermal filler system comprises 1 to 8 days. In some embodiments, the period of time after delivering the dermal filler system comprises 1 to 7 days. In some embodiments, the period of time after delivering the dermal filler system comprises 1 to 6 days. In some embodiments, the period of time after delivering the dermal filler system comprises 1 to 5 days. In some embodiments, the period of time after delivering the dermal filler system comprises 1 to 4 days. In some embodiments, the period of time after delivering the dermal filler system comprises 1 to 3 days. In some embodiments, the period of time after delivering the dermal filler system comprises 1 to 2 days.

In some embodiments, chronic inflammation may be characterized as the slow, long-term inflammation lasting for an extended period of time after the delivery or administration of the dermal filler system. In some embodiments, the chronic inflammation can last for a period of months to years. In some embodiments, the intentional foreign body response caused by the administration of biostimulators, as discussed above, results in chronic inflammation at the tissue site. In some embodiments, methods of delivering dermal filler systems described herein minimize such chronic inflammation while filling the tissue site with new tissue. In some embodiments, filling comprises forming new tissue within the cell matrix at the tissue site. In some embodiments, the new tissue has a persistence time in vivo at tissue site similar to endogenous tissue surrounding the tissue site. In some embodiments, a granuloma may be a small area of inflammation at or around a tissue site of a subject. In some embodiments, the granuloma may be a small area/cluster of white blood cells and other tissue at the area of inflammation. In some embodiments, the intentional foreign body response caused by the administration of biostimulators, as discussed above, results in granuloma formation at or around the tissue site. In some embodiments, methods of delivering dermal filler systems described herein minimize such granuloma formation while filling the tissue site with new tissue. In some embodiments, scar tissue is characterized by fibrous tissue having a harder and more brittle composition than normal tissue. In some embodiments, the intentional foreign body response caused by the administration of biostimulators, as discussed above, results in scar tissue formation at the tissue site. In some embodiments, methods of delivering dermal filler systems described herein minimize such scar tissue formation while filling the tissue site with new tissue. In some embodiments, the dermal filler systems disclosed herein minimize such scar tissue formation at least because the new tissue that is formed within the covalently stabilized scaffold mimics the tissue at or surrounding the tissue site. For example, there is less Type I collagen and more Type III collagen deposited in the cell matrix within the covalently stabilized scaffold (forming the basis of the new tissue) than scar tissue. In some embodiments, the nodules may be sites of abnormal tissue growths. In some embodiments, the intentional foreign body response caused by the administration of biostimulators, as discussed above, results in nodule formation at the tissue site. In some embodiments, methods of delivering dermal filler systems described herein minimize such nodule formation while filling the tissue site with new tissue. In some embodiments, the foreign body response is measured by detecting an amount of granulomas at the tissue site with histological analysis and comparing the amount of granulomas at the tissue site with a reference tissue that does not contain the dermal filler system. In some embodiments, the foreign body response is measured by detecting an amount of scar tissue at the tissue site with histological analysis and comparing the amount of scar tissue at the tissue site with a reference tissue that does not contain the dermal filler system. In some embodiments, the foreign body response is measured by detecting an amount of nodules at the tissue site with histological analysis and comparing the amount of nodules at the tissue site with a reference tissue that does not contain the dermal filler system. In some embodiments, foreign body response is measured by detecting chronic inflammation at the tissue site with histological analysis. In some embodiments, the foreign body response is measured by detecting an amount of multinucleate giant cells (MNGC) (e.g., fusion of monocytes or macrophages) present at the tissue site with histological analysis and comparing the amount of MNGCs at the tissue site with a reference tissue that does not contain the dermal filler formulation.

In some embodiments, methods of delivering the dermal filler system disclosed herein under conditions sufficient to deposit an amount or type of collagen in the cell matrix at the tissue site mimicking endogenous tissue at or surrounding the tissue site. In some embodiments, methods comprise depositing an amount or type of collagen in the cell matrix at the tissue site mimicking endogenous tissue at or surrounding the tissue site. In some embodiments, the cell matrix comprises an amount or a type of collagen mimicking endogenous tissue at the tissue site. In some embodiments, the type of collagen comprises Type I collagen, Type III collagen, or a combination thereof. In some embodiments, Type I collagen is present with Type III collagen in a ratio of less than or equal to about 10:1. In some embodiments, Type I collagen is present with Type III collagen in a ratio of less than or equal to about 6:1. In some embodiments, Type I collagen is present with Type III collagen in a ratio of about 5:1 or less. In some embodiments, Type I collagen is present with Type III collagen in a ratio of about 1:1 to about 10:1. In some embodiments, Type I collagen is present with Type III collagen in a ratio of about 1.5:1 to about 9.5:1. In some embodiments, Type I collagen is present with Type III collagen in a ratio of about 2:1 to about 9:1. In some embodiments, Type I collagen is present with Type III collagen in a ratio of about 2.5:1 to about 8.5:1. In some embodiments, Type I collagen is present with Type III collagen in a ratio of about 3:1 to about 8:1. In some embodiments, Type I collagen is present with Type III collagen in a ratio of about 3.5:1 to about 7.5:1. In some embodiments, Type I collagen is present with Type III collagen in a ratio of about 4:1 to about 7:1. In some embodiments, Type I collagen is present with Type III collagen in a ratio of about 4.5:1 to about 6.5:1. In some embodiments, Type I collagen is present with Type III collagen in a ratio of about 5:1 to about 6:1. Type I collagen may be an indicator of scar tissue or a foreign body response having taken place in the subject. In some embodiments, the lower the ratio of Type I collagen to Type III collagen, the more the foreign body response has been minimized. In some embodiments, methods comprise minimizing the ratio of Type I collagen to Type III collagen such that new tissue can be built within a subject, making new tissue with the characteristics disclosed herein while avoiding the harms disclosed herein. For example, methods comprise filling at least a part of the tissue site, while minimizing, or avoiding altogether, the foreign body response elicited as a response to biostimulators. In some embodiments, filling comprises forming new tissue within the cell matrix at the tissue site. In some embodiments, the new tissue has a persistence time in vivo at tissue site similar to endogenous tissue surrounding the tissue site.

In some embodiments, methods of delivering the dermal filler system disclosed herein under conditions sufficient to form elastin at the tissue site. In some embodiments, methods comprise forming elastin at the tissue site. In some embodiments, the elastin persists at or around the tissue site following complete degradation of the covalently stabilized scaffold. The presence of elastin may indicate the absence of scars or scar tissue at the tissue site and may therefore be an indicator that the foreign body response has been minimized.

In some embodiments, the dermal filler formulation is biocompatible with tissue at the tissue site as determined by one or more techniques described by ISO standard 10993,the contents of which pertaining to these techniques are hereby incorporated by reference in their entirety.

### Methods of Treatment

In some embodiments, the methods further comprise treating the tissue site of the subject by delivering the dermal filler system to the tissue site. In some embodiments, treating the tissue site comprises improving an aesthetic quality of tissue at the tissue site. In some embodiments, improving the aesthetic quality of the tissue comprises tissue filling, dermal filling, removing wrinkles, repairing tissue, or correcting skin irregularities. In some embodiments, the treating the tissue site comprises treating one or more diseases or conditions of the tissue, such as for example, skin cancer.

### Skin Cancer

In some embodiments, the methods comprise treating cancer. In some embodiments, the cancer comprises skin cancer. In some embodiments, the skin cancer comprises basal cell carcinoma, cutaneous squamous cell carcinoma, melanoma, and merkel cell carcinoma. In some embodiments, methods comprise delivering an anti-cancer therapeutic agent to the subject. In some embodiments, the dermal filler system comprises the anti-cancer therapeutic agent. In some embodiments, the microgel particles of the dermal filler system comprise the anti-cancer therapeutic agent (e.g., drug-eluding microgel particles). In some embodiments, the microgel particles elude the anti-cancer therapeutic agent in vivo at the tissue site. In some embodiments, the dermal filler system is formulated with the anti-cancer therapeutic agent. In some embodiments, the anti-cancer therapeutic agent comprises or is an anti-cancer therapeutic agent. In some embodiments, the anti-cancer therapeutic agent comprises or is a biologic. In some embodiments, the anti-cancer therapeutic agent comprises cisplatin, 5-fluorouracil (5-FU), Aldara (Imiquimod), Cemiplimab-rwlc, Efudex (Fluorouracil—Topical), Erivedge (Vismodegib), 5-FU (Fluorouracil—Topical), Fluorouracil—Topical, Imiquimod, Libtayo (Cemiplimab-rwlc), Odomzo (Sonidegib), Sonidegib, Vismodegib, Cemiplimab-rwlc, Keytruda (Pembrolizumab), Libtayo, Pembrolizumab, Aldesleukin, Binimetinib, Braftovi (Encorafenib), Cobimetinib, Cotellic (Cobimetinib), Dabrafenib, Dacarbazine, DTIC-Dome (Dacarbazine), Encorafenib, IL-2 (Aldesleukin), Imlygic (Talimogene Laherparepvec), Interleukin-2 (Aldesleukin), Intron A (Recombinant Interferon Alfa-2b), Ipilimumab, Keytruda (Pembrolizumab), Kimmtrak (Tebentafusp-tebn), Mekinist (Trametinib Dimethyl Sulfoxide), Mektovi (Binimetinib), Nivolumab, Nivolumab and Relatlimab-rmbw, Opdivo (Nivolumab), Opdualag (Nivolumab and Relatlimab-rmbw), Peginterferon Alfa-2b, Pembrolizumab, Proleukin (Aldesleukin), Recombinant Interferon Alfa-2b, Sylatron (Peginterferon Alfa-2b), Tafinlar (Dabrafenib), Talimogene Laherparepvec, Tebentafusp-tebn, Trametinib Dimethyl Sulfoxide, Vemurafenib, Yervoy (Ipilimumab), Zelboraf (Vemurafenib), Avelumab, Bavencio (Avelumab), or combinations thereof.

Disclosed herein are methods of treating skin cancer (e.g., basal cell carcinoma) in a subject, the method comprising administering one or more anti-cancer therapeutic agents and a microgel system to the subject. In some embodiments, the anti-cancer therapeutic agent comprises Aldara (Imiquimod), Cemiplimab-rwlc, Efudex (Fluorouracil—Topical), Erivedge (Vismodegib), 5-FU (Fluorouracil—Topical), Fluorouracil—Topical, Imiquimod, Libtayo (Cemiplimab-rwlc), Odomzo (Sonidegib), Sonidegib, Vismodegib, or combinations thereof. In some embodiments, methods comprise treating skin cancer (e.g., cutaneous squamous cell carcinoma) by administering one or more anti-cancer therapeutic agents comprising Cemiplimab-rwlc, Keytruda (Pembrolizumab), Libtayo, Pembrolizumab, or combinations thereof. In some embodiments, methods comprise treating skin cancer (e.g., melanoma) by administering one or more anti-cancer therapeutic agent comprising Aldesleukin, Binimetinib, Braftovi (Encorafenib), Cobimetinib, Cotellic (Cobimetinib), Dabrafenib, Dacarbazine, DTIC-Dome (Dacarbazine), Encorafenib, IL-2 (Aldesleukin), Imlygic (Talimogene Laherparepvec), Interleukin-2 (Aldesleukin), Intron A (Recombinant Interferon Alfa-2b), Ipilimumab, Keytruda (Pembrolizumab), Kimmtrak (Tebentafusp-tebn), Mekinist (Trametinib Dimethyl Sulfoxide), Mektovi (Binimetinib), Nivolumab, Nivolumab and Relatlimab-rmbw, Opdivo (Nivolumab), Opdualag (Nivolumab and Relatlimab-rmbw), Peginterferon Alfa-2b, Pembrolizumab, Proleukin (Aldesleukin), Recombinant Interferon Alfa-2b, Sylatron (Peginterferon Alfa-2b), Tafinlar (Dabrafenib), Talimogene Laherparepvec, Tebentafusp-tebn, Trametinib Dimethyl Sulfoxide, Vemurafenib, Yervoy (Ipilimumab), Zelboraf (Vemurafenib), or combinations thereof. In some embodiments, methods comprise treating skin cancer (e.g., merkel cell carcinoma) by administering one or more anti-cancer therapeutic agent comprising Avelumab, Bavencio (Avelumab), Keytruda (Pembrolizumab), Pembrolizumab, or combinations thereof.

### Aesthetic Treatments

Disclosed herein are methods of treating a tissue site of a subject to improve an aesthetic quality of tissue at or around the tissue site. In some embodiments, methods comprise administering to the subject a dose of a dermal filler system disclosed herein. In some embodiments, the administering comprises injecting the dermal filler system into a tissue site of the subject. In some embodiments, the treating comprises reducing wrinkles, filling fine lines in the skin, bulking subdermal tissues, or any combination thereof. In some embodiments, the treating comprises filling moderate to severe facial wrinkles and skin folds, such as nasolabial folds (e.g., lines extending from the sides of the nose to the edges of the mouth) and perioral lines (e.g., small wrinkles in the skin around the mouth and lips). In some embodiments, the treating comprises filling of the lips, cheeks, chin, back of the hand, or a combination thereof. In some embodiments, the treating comprises the restoration and correction of signs of facial fat loss (lipoatrophy) in people with human immunodeficiency virus (HIV). In some embodiments, the treating comprises the correction of contour deficiencies, such as wrinkles and acne scars. In some embodiments, the one or more dermatological conditions comprises: acne scars, basal cell carcinoma, cellulitis, epidermolysis bullosa, melanoma, merkel cell carcinoma, scars, skin biopsy, skin cancer, squamous cell carcinoma, stretch marks, or any combination thereof. In the case of tissue filler or dermal filler applications for volume loss related to aging, lipoatrophy, lipodystrophy, dermal scarring, or superficial or deep rhytides, injection of the microgel particles directly into the dermis via needle or cannula may be used to improve tissue contour, tissue loss, or tissue displacement.

### Vocal Cord Augmentation

Disclosed herein are methods of augmenting the vocal cords. In some embodiments, methods comprise administering to the subject a dose of a dermal filler system disclosed herein. In some embodiments, the administering comprises injecting the dermal filler system into the vocal cords of the subject. In some embodiments, the administering comprises injecting the dermal filler system into the superficial lamina propria or phonatory epithelium of the vocal cords. In some embodiments, the treating comprises the restoration of the subject's voice, the restoration of pliability to the superficial lamina propria, or the reduction of hoarseness of the subject's voice. In some embodiments, the subject has scarred and/or stiff vocal folds.

### Combination Treatments

In some embodiments, the methods comprise administering to the subject one or more additional agent (e.g., therapeutic agent) such as local anesthetics (e.g., lidocaine), pain medications, anti-inflammatory agents, anti-cancer therapeutic agent, or others that can provide a therapeutic or aesthetic benefit at the site of administration. In some embodiments, the microgel particles comprise one or more additional agents (e.g., drug-eluting microgel particles). In some embodiments, the microgel particles elute the one or more active agents in situ. In some embodiments, the dermal filler system is formulated with the one or more active agents. In some embodiments, the dermal filler system is not formulated with the one or more active agents, and the one or more active agents is administered separately from the dermal filler system. In some embodiments, the dermal filler system and the one or more additional active agents is administered to the subject sequentially. In some embodiments, the dermal filler system and the one or more additional active agents is administered to the subject substantially simultaneously.

In some embodiments, the therapeutic agent comprises a pain medication, a local anesthetic, an anti-inflammatory medication, an anti-fibrotic medication, or an antibiotic. In some embodiments the local anesthetic is ester based. In some embodiments, the ester based local anesthetic comprises benzocaine, chloroprocaine, procaine, proparacaine, tetracaine, amylocaine, or oxybuprocaine, or any combination thereof. In some embodiments, the local anesthetic is amide based. In some embodiments, the amide based local anesthetic comprises articaine, bupivacaine, dibucaine, etidocaine, levobupivacaine, lidocaine, mepivacaine, prilocaine, ropivacaine, sameridine, tonicaine, or cinchocaine, or any combination thereof. In some embodiments, the local anesthetic is or comprises lidocaine. In some embodiments, the local anesthetic consists of lidocaine. In some embodiments, the pain medication comprises codeine, fentanyl, hydrocodone, hydromorphone, meperidine, morphine, oxycodone, or tramadol, or any combination thereof. In some embodiments, the anti-inflammatory medication is a non-steroidal anti-inflammatory drug (NSAID) or a steroid. In some embodiments, the NSAID comprises ibuprofen or naproxen. In some embodiments, the steroid comprises a corticosteroid. In some embodiments, the antibiotic comprises dicloxacillin, erythromycin, or tetracycline. In some embodiments, the anti-fibrotic medication comprises pentoxifylline.

In some embodiments, methods comprise administering lidocaine at a concentration comprising about 1.0 milligrams per microliter (mg/mL) to about 5.0 mg/mL. In some embodiments, methods comprise administering lidocaine at a concentration comprising about 3.0 mg/mL. In some embodiments, the local anesthetic consists of lidocaine. In some embodiments, methods comprise administering lidocaine at a concentration of about 1.0 mg/mL to about 5.0 mg/mL. In some embodiments, methods comprise administering lidocaine at a concentration of about 3.0 mg/mL. In some embodiments, methods comprise administering the local anesthetic at a concentration of about 0.5 mg/mL to about 20.0 mg/mL. In some embodiments, methods comprise administering the local anesthetic at a concentration of about 1.0 mg/mL to about 19.5 mg/mL. In some embodiments, methods comprise administering the local anesthetic at a concentration of about 1.5 mg/mL to about 19.0 mg/mL. In some embodiments, methods comprise administering the local anesthetic at a concentration of about 2.0 mg/mL to about 18.5 mg/mL. In some embodiments, methods comprise administering the local anesthetic at a concentration of about 2.5 mg/mL to about 18.0 mg/mL. In some embodiments, methods comprise administering the local anesthetic at a concentration of about 3.0 mg/mL to about 17.5 mg/mL. In some embodiments, methods comprise administering the local anesthetic at a concentration of about 3.5 mg/mL to about 17.0 mg/mL. In some embodiments, methods comprise administering the local anesthetic at a concentration of about 4.0 mg/mL to about 16.5 mg/mL. In some embodiments, methods comprise administering the local anesthetic at a concentration of about 4.5 mg/mL to about 16.0 mg/mL. In some embodiments, methods comprise administering the local anesthetic at a concentration of about 5.0 mg/mL to about 15.5 mg/mL. In some embodiments, methods comprise administering the local anesthetic at a concentration of about 5.5 mg/mL to about 15.0 mg/mL. In some embodiments, methods comprise administering the local anesthetic at a concentration of about 6.0 mg/mL to about 14.5 mg/mL. In some embodiments, methods comprise administering the local anesthetic at a concentration of about 6.5 mg/mL to about 14.0 mg/mL. In some embodiments, methods comprise administering the local anesthetic at a concentration of about 7.0 mg/mL to about 13.5 mg/mL. In some embodiments, methods comprise administering the local anesthetic at a concentration of about 7.5 mg/mL to about 13.0 mg/mL. In some embodiments, methods comprise administering the local anesthetic at a concentration of about 8.0 mg/mL to about 12.5 mg/mL. In some embodiments, methods comprise administering the local anesthetic at a concentration of about 8.5 mg/mL to about 12.0 mg/mL. In some embodiments, methods comprise administering the local anesthetic at a concentration of about 9.0 mg/mL to about 11.5 mg/mL. In some embodiments, methods comprise administering the local anesthetic at a concentration of about 9.5 mg/mL to about 11.0 mg/mL. In some embodiments, methods comprise administering the local anesthetic at a concentration of about 9.5 mg/mL to about 10.5 mg/mL.

### Methods of Producing a Dermal Filler System

Disclosed herein, in some embodiments, are methods of producing the dermal filler systems disclosed herein and the components thereof. In some embodiments, the methods comprise synthesizing the microgel particles from raw materials. In some embodiments, the methods comprise fine-tuning the mechanical properties of the microgel particles, dermal filler system, or resulting porous covalently stabilized scaffold. In some embodiments, the methods comprise purifying the microgel particles. In some embodiments, the methods comprise formulating the microgel particles into a dermal filler system or formulation. In some embodiments, the methods further comprise sterilizing the dermal filler system or formulation.

### Synthesizing Microgel Particles

Disclosed herein are methods of producing a microgel particle disclosed herein, which comprise combining raw materials (e.g., polymer, functional groups, peptides, etc.) under conditions sufficient for the individual microgel particles to form. In some embodiments, the conditions sufficient for microgel particles to form may comprise an aqueous buffer with pH ranging from 7 to 9. By way of non-limiting examples, the buffer may be phosphate buffered saline (PBS), HEPES, or Triethanolamine (TEOA). In some embodiments, the reaction may be quenched by the addition of an acid or a base to stop the reaction at a specified time after mixing and creating the water-in-oil emulsion. The reaction quenching molecule could be added to the oil phase and diffuse into the aqueous phase to quench the reaction occurring in that aqueous phase. In some embodiments, the reaction may be quenched by adding a maleimide to react with the remaining thiols. In some embodiments, the reaction may be quenched by adding an oxidizing agent to oxidize the thiols.

In some embodiments, microgel particles may be synthesized using a microfluidic device (e.g., one particle at a time per channel). In some embodiments, the microgel particles may be synthesized by water-in-oil emulsion as described in greater detail herein. In some embodiments, the microgel particles may be synthesized by water-in-oil emulsion with mechanical stirring. In some embodiments, the microgel particles may be synthesized by water-in-oil emulsion using a static mixer. In some embodiments, the microgel particles may be synthesized using in-line flow-through synthesis. In some embodiments, the microgel particles may be synthesized using a parallel production method (multiple particles at a time per channel or multiple channels in parallel).

In some embodiments, methods comprise synthesizing microgel particles by a water-in-oil emulsion process. In some embodiments, the methods begin with obtaining an oil or an oil mixture. By way of non-limiting example, the oil may be a light mineral oil (LMO), a heavy mineral oil (HMO) or a fluorinated oil. In some embodiments, oil mixtures comprise a surfactant. In some embodiments, different surfactants can be employed. In some embodiments, the surfactant may be a nonionic surfactant. Non-limiting examples of nonionic surfactants are Span80, Span20, Tween20, Tween40, Tween60, Tween80, and tocopheryl polyethylene glycol 1000 succinate (TPGS). In some embodiments, the surfactant may be an anionic surfactant. In some embodiments, the surfactant may be a fluorinated surfactant. Non-limiting examples of anionic surfactants are sodium dodecyl sulfate (SDS), sodium lauryl ether sulfate (SLES), and perfluorooctanesulfonate. In some embodiments, the surfactant may be a cationic surfactant. Non-limiting examples of cationic surfactants are cetyltrimethylammonium bromide (CTAB), and hexadecylpyridium bromide. In some embodiments, the surfactant may be an amphoteric surfactant. Non-limiting examples of amphoteric surfactants are betaine citrate, lauryl betaine, sodium, and (carboxymethyl) dimethyloleyl ammonium hydroxide. In some embodiments, the concentration of the surfactant may vary from 0.01 to 5% v/v.

In some embodiments, methods comprise adding the surfactant to the oil. In some embodiments, methods comprise adding the surfactant to the oil prior to the addition of an aqueous solution/mixture to the oil. In some embodiments, methods comprise adding the surfactant to an aqueous solution/mixture described herein. In some embodiments, having a surfactant in the aqueous phase is beneficial because if the surfactant has a high-water solubility it is easy to remove during purification.

In some embodiments, the oil or oil mixture may be added to a bioreactor vessel through a micron filter and stirred. In some embodiments, the bioreactor vessel contains a volume from about 100 milliliters to about 1 liter. In some embodiments, the bioreactor vessel contains a volume from about 1 liter to about 10 liters. In some embodiments, the bioreactor vessel contains a volume from about 10 liters to about 100 liters. In some embodiments, the bioreactor vessel contains a volume from about 100 liters to about 1000 liters. In some embodiments, the bioreactor vessel contains a volume from about 100 liters to about 10,000 liters. In some embodiments, the bioreactor vessel contains a volume from about 10 liters to about 1000 liters. In some embodiments, the bioreactor vessel contains a volume from about 1000 liters to about 10,000 liters. In some embodiments, the micron filter has a pore size of about 0.1 μm to about 1 μm. In some embodiments, the micron filter has a pore size of about 0.2 μm.

In some embodiments, the oil or oil mixture may be added to a static mixer through a micron filter and stirred. In some embodiments, the static mixer contains a volume from about 100 milliliters to about 1 liter. In some embodiments, the static mixer contains a volume from about 1 liter to about 10 liters. In some embodiments, the static mixer contains a volume from about 10 liters to about 100 liters. In some embodiments, the static mixer contains a volume from about 100 liters to about 1000 liters. In some embodiments, the static mixer contains a volume from about 100 liters to about 10,000 liters. In some embodiments, the static mixer contains a volume from about 10 liters to about 1000 liters. In some embodiments, the static mixer contains a volume from about 1000 liters to about 10,000 liters. In some embodiments, the micron filter has a pore size of about 0.1 μm to about 1 μm. In some embodiments, the micron filter has a pore size of about 0.2 μm.

In some embodiments, methods of synthesizing microgel particles comprise providing one or more polymers as disclosed herein (e.g., in a solution). In some embodiments, the one or more polymers comprise PEG and HA. In some embodiments, the PEG and HA are provided in a molecular weight as disclosed herein.

In some embodiments, methods of synthesizing microgel particles comprise modifying the one or more polymers disclosed herein by attaching one or more functional groups. In some embodiments, the PEG and HA are modified by attaching thiol and vinyl sulfone functional groups. In some embodiments, the HA is modified with thiol and the PEG is modified with vinyl sulfone (VS).

In some embodiments, methods of synthesizing microgel particles comprise mixing the one or more modified polymers in a solution. In some embodiments, the thiolated HA is mixed with the PEG-VS. In some embodiments, the functional groups react to form a hydrogel (e.g., hydrogel mesh). In some embodiments, the functional groups react by a Michael addition reaction (e.g., thiol-ene Michael addition reaction). In some embodiments, methods may comprise filtering the solution. In some embodiments, the solution may comprise a peptide (e.g., cell adhesive peptide as disclosed herein). In some embodiments, the solution may comprise a buffer or buffering agent. In some embodiments, the solution may comprise a base catalyst.

In some embodiments, methods of synthesizing microgel particles comprise the methods disclosed in U.S. Pat. No. 10,668,185, which is incorporated herein by reference in its entirety.

### Fine-Tuning Mechanical Properties

Disclosed herein, in some embodiments, are methods of modulating the physical characteristics of the microgel particles, the covalently stabilized scaffolds, the dermal filler systems, or any combination thereof. In some embodiments, how the physical haracteristics are modulated will depend on the mode of delivery, tissue type and a location of the tissue site, desired therapeutic or aesthetic outcome, and the subject. In some embodiments, the physical characteristics may be altered depending on the tissue site, the mode of administration, the desired biocompatibility, or any combination thereof. In some embodiments, the physical characteristic is a mechanical property of the microgel particles, the covalently stabilized scaffolds, the dermal filler systems, or any combination thereof.

In some embodiments, methods comprise modulating the viscosity of the hydrogel, rate of degradation of the covalently stabilized scaffold, the volume fraction of the microgel particles, the pH of the microgel particles, the pH of the annealing agent solution, the pH of the covalently stabilized scaffold, the degree of substitution of the polymer, the elastic compressive modulus of the covalently stabilized scaffold, the storage modulus of the covalently stabilized scaffold, the weight percent of the polymers, the molar ratio of the functional groups, the molecular weight of the polymer, the molecular weight of the annealing agent, the additional agents (e.g., therapeutic agents), or a combination thereof. In some embodiments, methods comprise reducing the viscosity of the hydrogel, rate of degradation of the covalently stabilized scaffold, the volume fraction of the microgel particles, the pH of the microgel particles, the pH of the annealing agent, the pH of the covalently stabilized scaffold, the degree of substitution of the polymer, the elastic compressive modulus of the covalently stabilized scaffold, the storage modulus of the covalently stabilized scaffold, the weight percent of the polymer, the molar ratio of the functional groups, the molecular weight of the polymer, the molecular weight of the annealing agent, the additional agents (e.g., therapeutic agents), or a combination thereof. In some embodiments, methods comprise increasing the viscosity of the hydrogel, rate of degradation of the covalently stabilized scaffold, the volume fraction of the microgel particles, the pH of the microgel particles, the pH of the annealing agent, the pH of the covalently stabilized scaffold, the degree of substitution of the polymer, the elastic compressive modulus of the covalently stabilized scaffold, the storage modulus of the covalently stabilized scaffold, the weight percent of the polymer, the molar ratio of the functional groups, the molecular weight of the polymer, the molecular weight of the annealing agent, the additional agents (e.g., therapeutic agents), or a combination thereof.

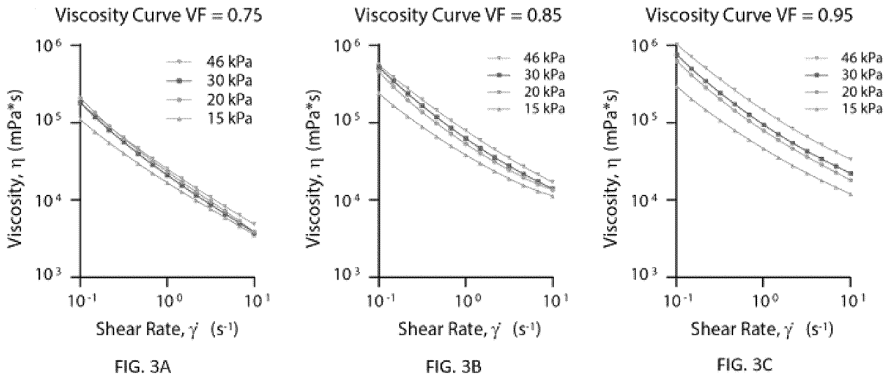

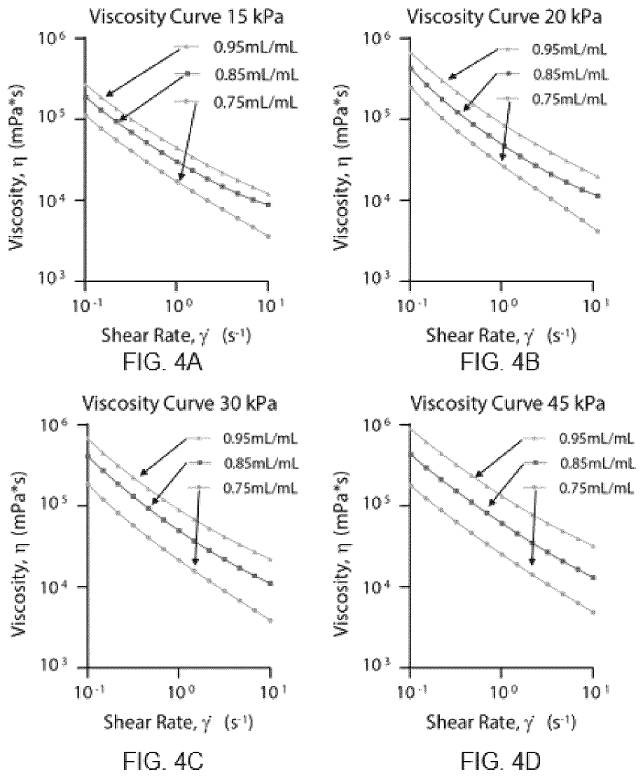

In some embodiments, methods comprise modulating (e.g., increasing or decreasing) the viscosity of the hydrogel or the elastic compressive modulus of the covalently stabilized scaffold. In some embodiments, methods comprise modulating the volume fraction of the microgel particles. In some embodiments, modulating the volume fraction of the microgel particles comprises modulating the percent concentration of microgel particles in the hydrogel. As shown in FIGS. 3A-3C and FIGS. 4A-4D, increasing the volume fraction of the microgel particles can lead to a higher viscosity of the suspension of microgel particles hydrogel to be annealed into a covalently stabilized scaffold. As shown in FIGS. 3A-3C and FIGS. 4A-4D, decreasing the volume fraction of the microgel particles can lead to a lower viscosity of the hydrogel to be annealed into a covalently stabilized scaffold. In some embodiments, methods comprise modulating the volume fraction of the microgel particles in a range of 0.75 mL/mL to 0.95 mL/mL to achieve a viscosity of the hydrogel of 1,000 to 1,000,000 mPa*s. As shown in FIG. 5, increasing the volume fraction of the microgel particles can lead to a higher elastic compressive modulus of the covalently stabilized scaffold. As shown in FIG. 5, decreasing the volume fraction of the microgel particles can lead to a lower elastic compressive modulus of the covalently stabilized scaffold. In some embodiments, methods comprise modulating the volume fraction of the microgel particles in a range of 0.75 mL/mL to 0.95 mL/mL to achieve an elastic compressive modulus of the covalently stabilized scaffold of about 1,000 Pa to about 17,000 Pa.

In some embodiments, methods comprise modulating the elastic modulus of the microgel particles. In some embodiments, modulating the elastic modulus is achieved by modulating the molar ratio of the crosslinkers, polymers (e.g., co-polymers), or combination thereof. As shown in FIGS. 3A-3C and FIGS. 4A-4D, increasing the elastic modulus of the microgel particles can lead to a higher viscosity of the hydrogel to be annealed into a covalently stabilized scaffold. As shown in FIGS. 3A-3C and FIGS. 4A-4D, decreasing the elastic modulus of the microgel particles can lead to a lower viscosity of the hydrogel to be annealed into a covalently stabilized scaffold. In some embodiments, methods comprise modulating the elastic modulus of the microgel particles in a range of 15 kPa to 46 kPa to achieve a viscosity of the hydrogel of 1,000 to 1,000,000 mPa*s. As shown in FIG. 5, increasing the elastic modulus of the microgel particles can lead to a higher elastic compressive modulus of the covalently stabilized scaffold. As shown in FIG. 5, decreasing the elastic modulus of the microgel particles can lead to a lower elastic compressive modulus of the covalently stabilized scaffold. In some embodiments, methods comprise modulating the elastic modulus of the microgel particles in a range of 15 kPa to 46 kPa to achieve an elastic compressive modulus of the covalently stabilized scaffold of about 1,000 Pa to about 17,000 Pa.

In some embodiments, methods comprise modulating (e.g., increasing or decreasing) the volume fraction of the microgel particles in a range of 0.75 mL/mL to 0.95 mL/mL, and modulating the elastic modulus of the microgel particles in a range of 15 kPa to 46 kPa, to achieve a viscosity of the hydrogel of 1,000 to 1,000,000 mPa*s. In some embodiments, methods comprise modulating the volume fraction of the microgel particles in a range of 0.75 mL/mL to 0.95 mL/mL, and modulating the elastic modulus of the microgel particles in a range of 15 kPa to 46 kPa, to achieve an elastic compressive modulus of the covalently stabilized scaffold of about 1,000 Pa to about 17,000 Pa.

In some embodiments, methods comprise modulating (e.g., increasing or decreasing) the rate of degradation of the covalently stabilized scaffold. In some embodiments, the rate of degradation is altered depending on how long it is desired for the covalently stabilized scaffold to remain at the tissue site. In some embodiments, methods comprise altering the degradation pathway, altering the polymers (e.g., co-polymers) used to make up the microgel particles, or a combination thereof to alter the rate of degradation. In some embodiments, methods comprise altering the degradation pathways to one or more of oxidative degradation, enzymatic degradation, or hydrolytic degradation. In some embodiments, methods comprise synthesizing the microgel particles with PEG to decrease the rate of degradation of the covalently stabilized scaffold. In some embodiments, methods comprise synthesizing the microgel particles without PEG to increase the rate of degradation.

In some embodiments, methods comprise modulating (e.g., increasing or decreasing) the degree of substitution of the polymer. In some embodiments, modulating the degree of substitution is achieved by increasing or decreasing the amount of functional groups to be coupled to the microgel particles. In some embodiments, as shown in Table 3 below, modulating the molecular weights of the polymers (e.g., co-polymers) of the microgel particles alters the degree of substitution. In some embodiments, methods comprise measuring the degree of substitution using Ellman's assay.

In some embodiments, methods comprise modulating (e.g., increasing or decreasing) the elastic compressive modulus of the covalently stabilized scaffold. In some embodiments, modulating the concentration of functional groups (e.g., thiol and vinyl sulfone) included in the microgel particles can alter the elastic compressive modulus of the covalently stabilized scaffold. In some embodiments, modulating the molecular weight of the polymer(s) (e.g., co-polymer(s)) of the microgel particles can alter the concentration of functional groups (e.g., thiol and vinyl sulfone) included in the microgel particles. As shown in FIGS. 19A-19B, increasing the concentration of the functional groups (e.g., thiolated hyaluronic acid (SH-HA)) can increase the elastic compressive modulus of the covalently stabilized scaffold. As shown in FIGS. 19A-19B, decreasing the concentration of the functional groups (e.g., thiolated hyaluronic acid (SH-HA)) can decrease the elastic compressive modulus of the covalently stabilized scaffold. Also, as shown in FIGS. 19A-19B, increasing the molecular weight of the polymer(s) (e.g., hyaluronic acid) can increase the elastic compressive modulus of the covalently stabilized scaffold, and decreasing the molecular weight of the polymer(s) (e.g., hyaluronic acid) can decrease the elastic compressive modulus of the covalently stabilized scaffold. In some embodiments, methods comprise modulating the concentration of functional groups (e.g., thiol and vinyl sulfone) included in the gelation solution to a range of about 10 mg/mL to about 45 mg/mL to achieve an elastic compressive modulus of the covalently stabilized scaffold of about 100 Pa to about 140,000 Pa.

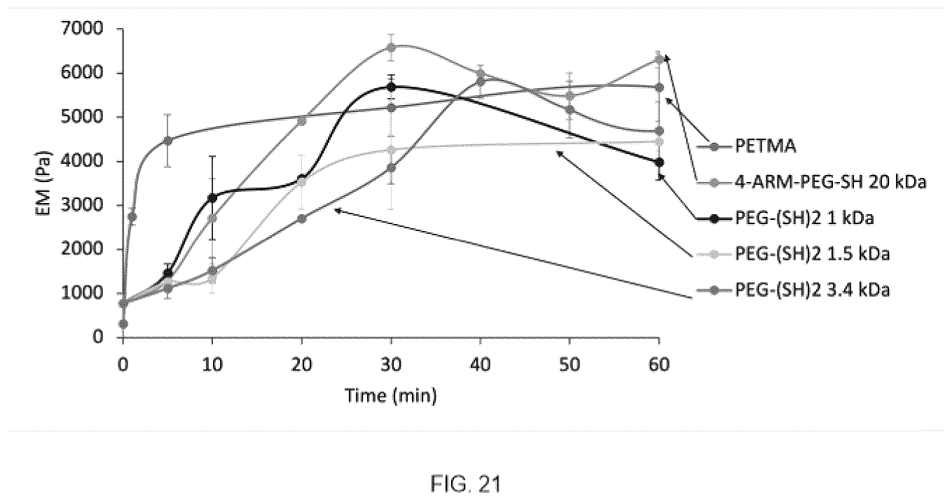

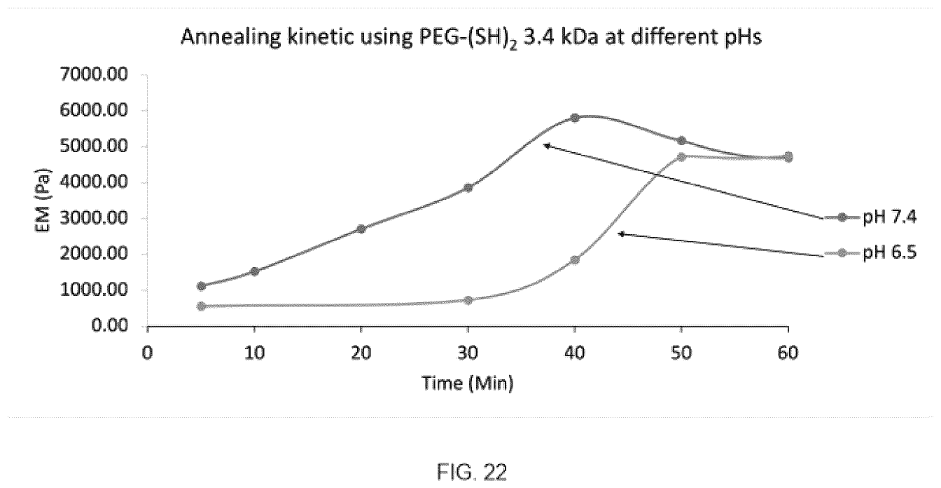

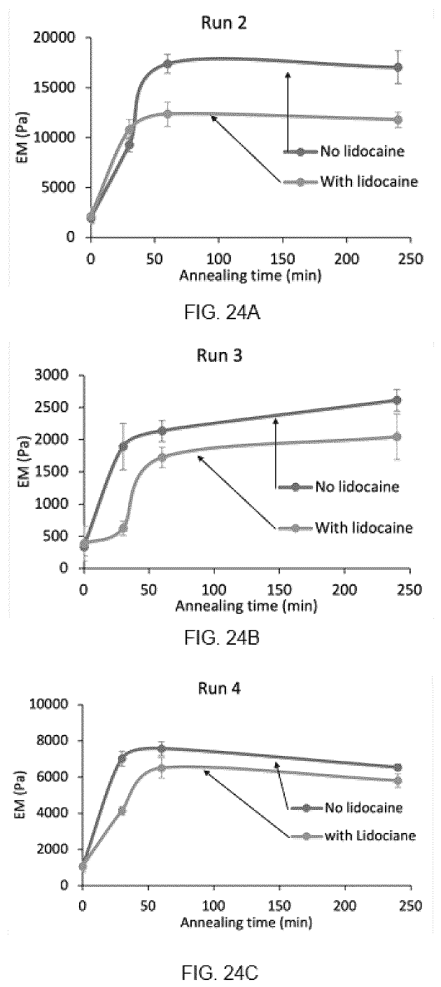

In some embodiments, methods comprise modulating (e.g., increasing or decreasing) how fast the covalently stabilized scaffold anneals. In some embodiments, altering the annealing agent can alter how fast the covalently stabilized scaffold anneals. In some embodiments, modulating the molecular weight of the annealing agent alters how fast the covalently stabilized scaffold anneals. As shown in FIG. 21, PEG-dithiol (PEG(SH)₂), 4-ARM-PEG-SH, and PETMA can be used to achieve an elastic compressive modulus of the covalently stabilized scaffold of about 4,000 Pa to about 7,000 Pa after about 60 minutes of annealing. In some embodiments, methods comprise modulating the pH of the annealing agent to alter how fast the covalently stabilized scaffold anneals. As shown in FIG. 22, increasing the pH of the annealing agent can increase how fast the covalently stabilized scaffold anneals, and decreasing the pH of the annealing agent can decrease how fast the covalently stabilized scaffold anneals. In some embodiments, methods comprise using a pH of the annealing at or below 6.5 to delay the start of the annealing reaction by 30 minutes or more. In some embodiments, methods comprise delivering a therapeutic agent (e.g., a local anesthetic (e.g., lidocaine)) to alter how fast the covalently stabilized scaffold anneals. As shown in FIGS. 24A-24C (each of FIGS. 24A, 24B, and 24C shows the results of different dermal filler systems, as described herein), local anesthetics can decrease how fast the covalently stabilized scaffold anneals, and can function in dermal filler systems with an elastic modulus range from about 1,500 Pa to about 18,000 Pa. In some embodiments, methods comprise delivering a therapeutic agent (e.g., a local anesthetic (e.g., lidocaine)) to achieve an elastic modulus of the covalently stabilized scaffold of about 2,000 Pa to about 12,000 Pa after about 250 minutes of annealing.

### Purifying Microgel Particles

In some embodiments, methods comprise purifying the microgel particles. In some embodiments, methods comprise synthesizing and purifying microgel particles simultaneously. In some embodiments, methods comprise purifying microgel particles after synthesizing the microgel particles. In some embodiments, purifying microgel particles comprises performing membrane separation of the microgel particles from unwanted components. In some embodiments, different types of filtration membranes may be used (e.g., hollow fiber membranes with different pore sizes, different lumen IDs, dialysis or flat sheet membrane). In some embodiments, membrane separation comprises tangential flow filtration (TFF). In some embodiments, membrane separation comprises ultrafiltration-diafiltration (UFDF). In some embodiments, membrane separation comprises microfiltration-diafiltration (MFDF). In some embodiments, membrane separation comprises hollow-fiber-diafiltration (HFDF). TFF generally comprises a membrane filtration and separation technique. TFF may be used herein to purify and concentrate microgel particles. TFF may comprise generating a feed stream of a solution of microgel particles that passes parallel to a membrane face. In some embodiments, one portion of the solution may pass through the membrane (permeate) while the remainder (retentate) is recirculated back to the feed reservoir. This system may be referred to as diafiltration. This system may allow molecules (in the permeate) smaller than the membrane pores to move toward and through the membrane while the larger molecules, such as the microgel particles, remain in the retentate. In some embodiments, the flow in the filtration system may be controlled by a peristaltic pump. In some embodiments, the flow in the filtration system may be controlled by a Quattroflow pump or any positive displacement pump. In some embodiments, the filtration system may be closed to surrounding environment. In some embodiments, the filtration system may be open to surrounding environment.

In some embodiments, methods of purifying may comprise removing excess oil from the microgel particles. In some embodiments, methods of purifying may comprise dispersing the particles in an alcohol solution. In some embodiments, the alcohol solution comprises alcohol and water, and the alcohol is present in the solution at a ratio greater than or equal to about 0.8:1. In some embodiments, the alcohol solution comprises alcohol and water, and the alcohol is present in the solution at a ratio greater than or equal to about 0.5:1, 0.6:1, 0.7:1, 0.8:1, 0.9:1, or 1:1. In some embodiments, the alcohol solution comprises alcohol and water, and the alcohol is present in the solution at a ratio less than or equal to about 0.5:1, 0.6:1, 0.7:1, 0.8:1, 0.9:1, or 1:1. In some embodiments, the alcohol solution comprises alcohol and water, and the alcohol is present in the solution at a ratio of about 0.5:1 to about 1:1. In some embodiments, the alcohol solution comprises alcohol and water, and the alcohol is present in the solution at a ratio of about 0.6:1 to about 0.9:1. In some embodiments, the alcohol solution comprises alcohol and water, and the alcohol is present in the solution at a ratio of about 0.7:1 to about 0.8:1. In some embodiments, methods of purifying may comprise removing excess oil and surfactant that are not miscible in water while keeping the particles (mainly composed of water) dispersed and sufficiently swollen and ensuring no particle aggregation. In some embodiments, methods of purifying may comprise slowly transferring the particles into an aqueous buffer while preventing the surfactant from precipitating. In some embodiments, transferring rate may be linked to the flux of filtrate passing through the membrane, and occur at a rate of about 1 to about 1000 LMH (liters/m²/h). In some embodiments, transferring may occur at a rate of about 100 to about 500 LMH. In some embodiments, transferring may occur at a rate of about 200 to about 300 LMH. This transition rate may be particularly important to ensure that a surfactant does not precipitate on to (and within) the microgel particles, rendering the particles unsuitable for a microporous scaffold. In some embodiments, the transition rate may achieve at least one of (i) particle hydrogel mesh swelling, which is a product of the affinity for certain solvents for a given hydrogel polymer backbone/crosslinker system, and (ii) solubility of the surfactant in the continuous phase outside of the particle.

In some embodiments, methods comprise concentrating the microgel particles in a solution or suspension. In some embodiments, methods comprise: pumping the microgel particles through a membrane filtration system while a continuous phase volume is removed; continually concentrating the microgel particles at a controlled membrane flux; and maintaining a wall shear stress inside the membrane filtration system. In some embodiments, the membrane filtration system is selected from tangential flow filtration (TFF), ultrafiltration-diafiltration (UFDF), microfiltration-diafiltration (MFDF), or hollow-fiber-diafiltration (HFDF). In some embodiments, the membrane flux is controlled between 100 and 1000 L/m²/h. In some embodiments, the wall shear stress is maintained between 100 s−1 and 10,000 s−1.

### Preserving Microgel Particles

Disclosed herein, in some embodiments, are methods of preserving the microgel particles, annealing agents, additional active agents, therapeutic agents, dermal filler systems or formulations, or a combination thereof. In some embodiments, the methods comprise preserving the microgel particles, annealing agents, additional active agents, therapeutic agents, or any combination thereof before formulating into a dermal filler system. In some embodiments, the preserving is performed prior to administration of the dermal filler system to a subject. In some embodiments, methods of preservation comprise lyophilization, cryodehydration, cryohibernation, or cryopreservation, or a combination thereof.

In some embodiments, the lyophilization of the microgel particles, annealing agents, therapeutic agents, or a combination thereof comprises the use of lyoprotectants for retaining the functionality of the microgel particles, annealing agents, therapeutic agents, or a combination thereof. Lyoprotectant comprises addition of reagents, salts, or additives that protects the microgel particles, annealing agents, therapeutic agents, or a combination thereof during the desiccation process. Common lyoprotectants include isopropanol, glycerol, trehalose, DMSO, methylcellulose, sucrose, antioxidants, human or animal serum proteins, and cellular stress proteins. Additionally, methods for increasing the transport of lyoprotectants inside the microgel particles, annealing agents, therapeutic agents, or a combination thereof in suspension can be utilized as a way of improving the viability and function of the microgel particles, annealing agents, therapeutic agents, or a combination thereof after lyophilization. These methods include electroporation, and the addition of reagents. In some embodiments, the lyophilized microgel particles, annealing agents, therapeutic agents, or a combination thereof, can be reconstituted for delivery to a tissue site of a subject. In some embodiments, reconstitution is accomplished by introducing a reconstitution medium to the lyophilized microgel particles, annealing agents, therapeutic agents, or a combination thereof.

In some embodiments, the microgel particles are flash frozen. In some embodiments, the microgel particles are flash frozen with liquid nitrogen. In some embodiments, the microgel particles are frozen at a temperature of at least about −100 C, −110 C, −120 C, −130 C, −140 C, −150 C, −160 C, −170 C, −180 C, −190 C or −200. In some embodiments, the microgel particles are frozen at a temperature of about −196 C. In some embodiments, the microgel particles are in a solution of at least about 80%, 85%, 90%, 95%, or 100% isopropanol.

In some embodiments, lyophilization occurs at a temperature of about −55 C. In some embodiments, lyophilization occurs at a temperature of less than about −50 C, −55 C, −60 C, −65 C, −70 C, −75 C, −80 C, −85 C, −90 C, −95 C, −100 C. In some embodiments, the volume fraction of the microgel particles duing lyophilization is less than about 10%, 20%, 30%, 40%, 50%, 60%, 70%, 80%, 90%, or 100%.

### Kits

Disclosed herein, in some embodiments, are kits useful for delivering the dermal filler systems disclosed herein. In some embodiments, the kits disclosed herein may be used to deliver the dermal filler system to a tissue site in a subject. In some embodiments, the kit comprises the dermal filler systems described herein, which can be used to perform the methods described herein. In some embodiments, the kit comprises the hydrogel particles and the annealing agent in separate containers. In some embodiments, the kit comprises the hydrogel and the annealing agent in a single container. In some embodiments, the kit also comprises a reconstitution medium as described herein to reconstitute a lyophilized dermal filler system (e.g., lyophilized hydrogel, annealing agent, or combination thereof).

Instructions for use may be included in the kit. Optionally, the kit also contains other useful components, such as, diluents, buffers, pharmaceutically acceptable carriers, syringes, catheters, applicators, pipetting or measuring tools, bandaging materials, or other useful paraphernalia. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit may be those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### EXAMPLES

The following examples are included for illustrative purposes only and are not intended to limit the scope of the invention.

### Example 1: Rheological Testing of Dermal Filler Systems

### Dermal Filler System Preparation

In this example, 14 different dermal filler systems were evaluated including: (1) 2 batches of aged manufacturing equivalent dermal filler systems, and (2) 12 batches formulated for having characteristic of device volume fraction (VF) concentration and elastic modulus of the microgel particles themselves (microgel particle stiffness).

In order to achieve 12 batches with varying parameters, four different bulk formulations of gel were made. These distinct formulations were created by titrating the relative concentrations of the MMP2-sensitive peptide crosslinker with the PEG-VS polymer of the microgel particles. Changes in this molar ratio (thiol/VS molar ratio) enabled deterministic selection of microgel particle stiffnesses ranging from 15 (kilopascal) kPa to 46 kPa. These microgel particle stiffnesses are measured by compressive testing by measuring the compressive elastic modulus (EM) of a macroscopic surrogate bulk gel made from the same mixture used to make the hydrogel microgel particle suspension. Table 1 presents all test article lot/batch information.

TABLE 1
Characterization Information of Material Used in Rheology Testing for this Example

| | | | | Xlinked EM | |
|---|---|---|---|---|---|
| | Microgel | | | after | |
| | particle Stiffness | | VF | Light Exposure | Product age at |
| Group | EM (Pa) | Lot No. | (mL/mL) | (Pa) | time of test |
| Bulk (1) | 15 kPa | 02-12-006 | 0.75 | 1739 | |
| | | 02-12-010 | 0.85 | 2254 | |
| | | 02-12-014 | 0.95 | 4250 | |
| Bulk (2) | 20 kPa | 02-12-004 | 0.75 | 2540 | |
| | | 02-12-008 | 0.85 | 3708 | |
| | | 02-12-012 | 0.95 | 5210 | |
| | | 02-12-005 | 0.75 | 1929 | |
| | | 02-12-009 | 0.85 | 5336 | |
| | | 02-12-013 | 0.95 | 8293 | |
| Bulk (4) | 46 kPa | 02-12-007 | 0.75 | 7907 | |
| | | 02-12-011 | 0.85 | 9007 | |
| | | 02-12-015 | 0.95 | 16287 | |
| Aged, | 21 kPa | DHR-020-010 | 0.86 | 5178 | 10 months |
| Released | 21 kPa | DHR-020-011 | 0.88 | 6236 | 10 months |
| Material | | | | | |

TABLE 2
Comparator or Standards Used in this Example

| Material | Vendor | Part # | Lot # | Expiration |
|---|---|---|---|---|
| Viscosity and Density Reference | Anton Paar | APN415 | 5191608 | 19 May 23 |
| Standard | | | | |

A modular compact rheometer (MCR102e, Anton Paar) was used to evaluate rheologic parameters of the materials tested in this example. The rheometer was filled with a parallel plate test system using 25 mm sandblasted plates. Sandblasted surfaces were selected to mitigate against any test article slip (previous data from testing indicated slip was occurring when using smooth plates). The sample chamber in the MCR102e was maintained at 20° C. for all testing in this example.

TABLE 3
Equipment Used

| Equipment | ID or Part # | Serial # |
|---|---|---|
| Modular Compact Rheometer | MCR102e (Anton Paar) | 83782848 |
| 25-mm diameter sandblasted | PP25/s (Anton Paar) | 82245 |
| measurement system | | |
| 1,000 μL positive displacement | 0015 | Q10092L |
| pipette | | |

### Methods

The MCR102e was powered on, and the sample chamber was allowed to equilibrate to 20° C. A series of baseline measurements was collected as part of the machine setup, which measured the resistance in the motor with no measurement system attached, followed by a measurement of the moment of inertia of the 25-mm diameter sandblasted upper plate measurement system. Prior to measuring any test samples, a final baseline reference test was conducted with the APN415 viscosity and density reference standard.

Microgel particle batches selected for testing were allowed to equilibrate to room temperature for >30 minutes before rheometric testing. Product was protected from light until it was prepared for testing.

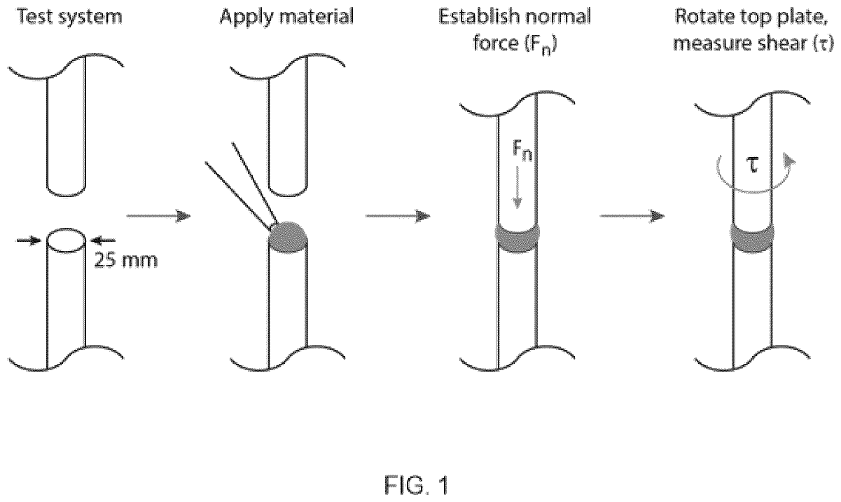

After cleaning the measurement system and bottom plate, ˜490 microliters (μL) of test article were loaded onto the center of the bottom plate using a 1,000 μL positive displacement pipet. The top plate was then lowered to a 1-mm gap from top to bottom plate to establish a normal force (Fr) necessary to ensure good readings during the flow curve measurement. Any excess test article was removed from the perimeter of the 1-mm plate-to-plate gap. A temperature equilibration period of 2 minutes was implemented for each test, whereby temperature was held at 20.00 (degrees Celsius) ° C.±0.05 as measured by a thermocouple residing in the lower plate. FIG. 1 illustrates the workflow for starting and conducting the flow curve measurements.

The apparent viscosity was measured at a series of 25 controlled shear rate (γ) observation points ranging from 0.1 s⁻¹ to 1,000 s⁻¹ with point measurement time ramping logarithmically from 10 s (@0.1 s⁻¹) down to 1 s (@1,000 s⁻¹). This time profile was chosen to decrease noise and enable more consistent measurements. Each product lot was measured in duplicate, and the coefficient of variation (CV) at each γ was calculated for each shear rate point. These point CVs were then averaged to give the total CV for each lot as a measure of reproducibility in the measurement. Measured torque T (N*m) was converted to shear stress τ(Pa) using equation 1 below and then to apparent viscosity η (Pa*s) using equation 2 below. The apparent viscosity was then plotted against the shear rates to produce a viscosity curve of η (Pa*s) vs γ (s⁻¹).

$$τ = \frac{T * r}{J} ( Shear Stress [ \frac{N}{{m}^{2}} ] = Torque [ N * m ] * \frac{radius [ m ]}{Polar} moment of intertia [ {m}^{4} ] ) \qquad ( 1 )$$

$$η = \frac{τ}{γ} ( Apparent Viscosity [ Pa * s ] = Shear stress [ N / {m}^{2} ] / Shear strain [ {s}^{- 1} ] ) \qquad ( 2 )$$

$$η = K ( T ) {γ}^{n - 1} ( Ostwald de - Waele Power Law ) \qquad ( 3 )$$

### Results

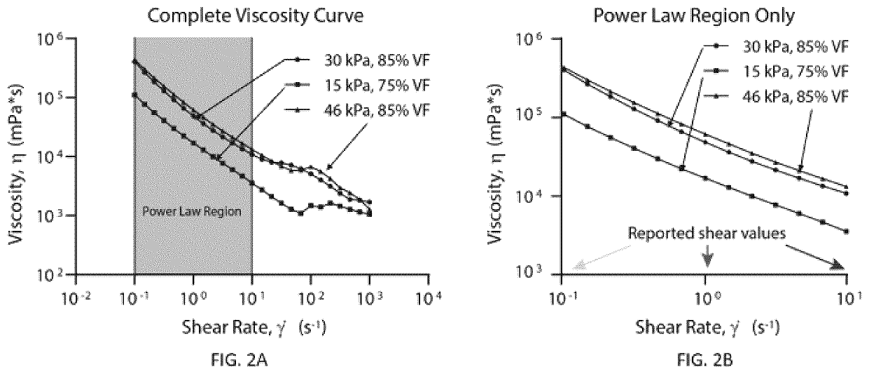

The viscosity curves for each lot appeared to show shear thinning behavior, meaning the apparent viscosity decreases as shear increases. On the log-log plot of viscosity η vs shear rate γ a linear regime was identified between 0.1 s⁻¹≤γ≤10 s⁻¹ (FIG. 2A). In this linear regime of the log-log plot (FIG. 2B), the Ostwald de-Waele power law equation, in its linear form, can be used to accurately calculate the viscosity η at a given shear rate, γ (equation 3). Within this region, three shear values were chosen to report viscosity (0.1, 1 and 10).

To determine the appropriateness of the regime for using the linearized Ostwald de-Waele equation, a power fit was performed for all lots tested within the region from 0.1 s⁻¹ to 10 s⁻¹. Within this power law region, N=13 points were used to calculate the values of η for each curve and perform the log/log regressions. All lots reported in Table 1 displayed an R²>0.98 for regression analysis (reported in Table 4), indicating the region is appropriate for this method of viscosity calculation. The slopes reported in Table 4 are equivalent to (n−1) in equation 3. All slope values were less than zero, requiring that n<1. This is indicative of a shear thinning behavior.

Table 4 shows the comprehensive results from all 14 batches testing in this example. Volume fraction (VF), microgel particle stiffness, elastic modulus (EM), and crosslinked EM after light exposure results are reported in the manufacturing records for each lot number. Reported viscosity at three shear values within the power law region are color-coded to correlate with the values highlighted in FIGS. 2A-2B, and FIG. 5.

TABLE 4

| | | | | | | | | | slope of |
|---|---|---|---|---|---|---|---|---|---|
| | | | Xlinked | Average Viscosity n | | | | | log-log |
| Group | | | EM | (Pa · s) @ three | | | | R² of | linear |
| (micro- | | | after light | shear rates γ in the | | | | log-log | regression |
| sphere | | VF | exposure | power law region | | | Average | linear | (log(n)/ |
| stiffness) | Lot No. | (mL/mL) | (Pa) | 0.1 s⁻¹ | 1.0 s⁻¹ | 10.0 s⁻¹ | CV % | regression | log(y)) |
| Bulk 1 | 02-12-006 | 0.75 | 1739 | 110.170 | 16.892 | 3.572 | 8.45% | 0.9971 | −0.7332 |
| 15 kPa | 02-12-010 | 0.85 | 2254 | 185.990 | 29.748 | 8.801 | 1.83% | 0.9881 | −0.6648 |
| | 02-12-014 | 0.95 | 4250 | 266.955 | 44.204 | 11.993 | 9.10% | 0.9922 | −0.6671 |
| Bulk 2 | 02-12-004 | 0.75 | 2540 | 209.900 | 23.340 | 3.925 | 2.29% | 0.9959 | −0.8415 |
| 20 kPa | 02-12-008 | 0.85 | 3708 | 354.280 | 41.436 | 10.497 | 4.16% | 0.9854 | −0.7562 |
| | 02-12-012 | 0.95 | 5210 | 551.865 | 74.137 | 17.869 | 0.75% | 0.9909 | −0.7338 |
| Bulk 3 | 02-12-005 | 0.75 | 1929 | 181.110 | 21.014 | 3.816 | 5.78% | 0.9956 | −0.8242 |
| 30 kPa | 02-12-009 | 0.85 | 5336 | 401.590 | 48.470 | 10.848 | 4.17% | 0.9912 | −0.7804 |
| | 02-12-013 | 0.95 | 8293 | 661.050 | 87.954 | 21.684 | 2.47% | 0.9901 | −0.7353 |
| Bulk 4 | 02-12-007 | 0.75 | 7907 | 178.685 | 25.453 | 4.919 | 5.24% | 0.9978 | −0.7752 |
| 46 kPa | 02-12-011 | 0.85 | 9007 | 433.290 | 61.405 | 13.224 | 2.38% | 0.9957 | −0.756 |
| | 02-12-015 | 0.95 | 16287 | 894.120 | 133.87 | 32.601 | 1.52% | 0.9935 | −0.7187 |
| Aged | DHR-020- | 0.86 | 6009 | 789.060 | 67.538 | 5.673 | 5.51% | 0.9998 | −1.0538 |
| Material | [symbol] | | | | | | | | |
| 21 kPa | DHR-020- | 0.88 | 5850 | 803.875 | 63.459 | 6.739 | 5.44% | 0.9985 | −1.0207 |
| | 011 | | | | | | | | |

[symbol]

indicates data missing or illegible when filed

### Measuring the Effects of Various Device Characteristics on Rheologic Parameters:

The following characteristics were evaluated to understand their effect on the rheological properties of the device: (1) Volume fraction (VF) of microgel particles, and (2) microgel particle stiffness.

Evaluation of the 12 batches with unique volume fractions and microgel particle stiffnesses enabled a basic understanding of the effects of each parameter on product rheology. FIGS. 3A-3C present the viscosity curves for each microgel particle stiffness, where FIG. 3A shows data from volume fractions of 75%, FIG. 31B shows data from volume fractions of 85%, and FIG. 3C shows data from volume fractions of 95%.

Analysis of the panels in FIG. 3A-3C illustrates that both volume fraction and microgel particle stiffness have an effect on product rheology within the power law region. A comparison of the panels indicates that microgel particle volume fraction may have a larger impact on product rheology compared to the stiffness. Further analysis of the viscosity curves was performed to investigate the functional dependence of each parameter on product rheology and is presented below.

FIGS. 4A-4D present the same data as in FIGS. 3A-3C, however grouped by microgel particle stiffness, with each curve representing a different volume fraction at the same microgel particle stiffness. Visualization of the viscosity curves when separated by product volume fraction (plotted for a given microgel particle stiffness) provides a clearer separation of viscosity curves.

As shown from FIGS. 3A-3C and FIGS. 4A-4D, both microgel particle stiffness and volume fraction have an effect on the product viscosity curves. The effect of microgel particle volume fraction and microgel particle stiffness on the crosslinked elastic modulus of the scaffolds after annealing are presented in FIG. 5 for reference. FIG. 5 illustrates the effects of volume fraction (VF) and microgel particle stiffness on the crosslinked elastic modulus (EM) after annealing. The shaded box in FIG. 5 highlights the design specification ranges already selected for VF and EM, in some embodiments described herein where the dermal filler comprises 4-ARM PEG-VS (20 kDa) and MMP-2 peptides in the microgel particles and Eosin Y and light for annealing. The output from this previous testing informed the best practices for formulating microgel particles (targeting a microgel particle stiffness of 15 kPa to 30 kPa) in order to ensure the crosslinked EM falls within the specified range (1,500≤EM≤10,000 Pa) when the product is formulated within the specified VF range (0.75≤VF≤0.95). Specifically, formulating to a microgel particle stiffness of 46 kPa may produce out of specification EM results.

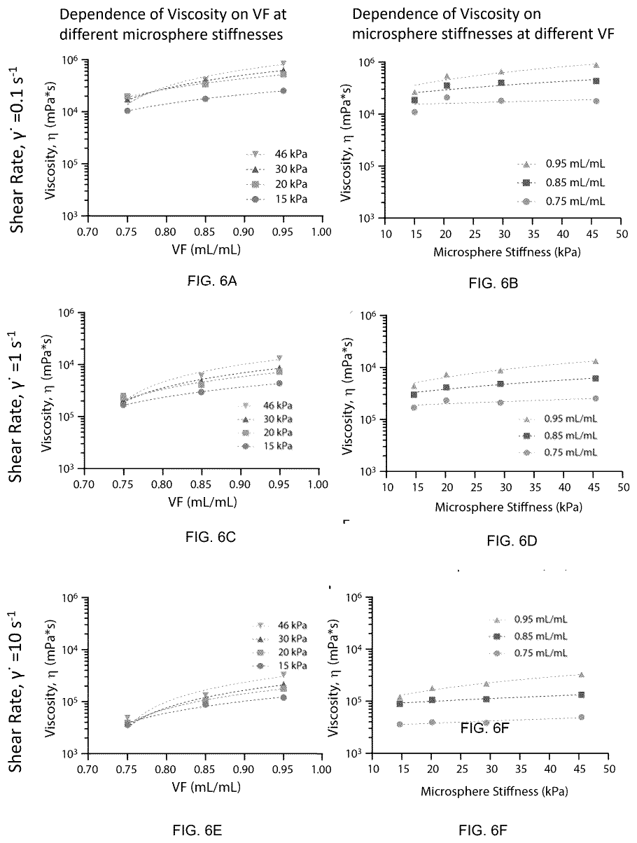

The summary plots in FIGS. 6A-6F illustrate the effects of microgel particle volume fraction and microgel particle stiffness on apparent viscosity across the shear range assessed (from 0.1 s⁻¹ to 10 s⁻¹).

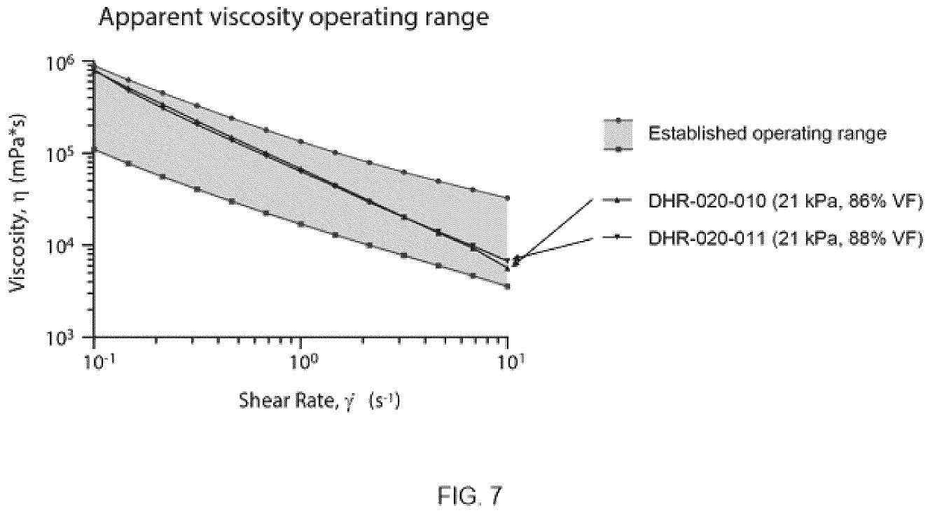

For ease of visualization, a rheologic range from the lowest and highest viscosity curves obtained from the different batches tested in this example are illustrated in FIG. 7. In addition, the viscosity curves of the two scaffolds that were previously manufactured and then aged at 5° C. for 10 months prior to rheologic testing are also plotted in FIG. 7 (black lines). The characteristics of these two lots are also presented in Table 1 and Table 4 above. This demonstrates that these aged batches fall within the operating range demonstrated in this example, and that scaffolds can be aged and still demonstrate predictable rheological parameters based on their volume fraction and microgel particle stiffness.

### Example 2: Dermal Filler Systems Comprising Hyaluronic Acid: Intradermal Injections in a Rat Model

In this example, dermal filler systems incorporating Hyaluronic acid (HA) as a component of the microgel particles were tested in intradermal injection in rat models, assessing tissue response of 30 days and volume change immediately after injection.

### Formulation

Here, the formulation strategy utilizes a 4-arm 20 kDa poly(ethylene glycol) (PEG) backbone that has been activated with 4 vinyl sulfone (VS) groups, at the end of each arm (PEG-VS). The PEG-VS is then mixed with a crosslinker—in the case of formulations 1-3 that crosslinker is a synthetic peptide of 16 amino acids that contains the cleavage sequence of matrix metalloprotease-2 (MMP-2). The peptide has been synthesized to have two cysteines with available thiols. When mixed with the PEG-VS activated polymer, the thiols and VS undergo a thiol-ene Michael addition reaction at pH >7 (kinetics of the reaction are governed by pH and reactant concentration). The PEG-VS is pre-reacted (before addition of the MMP-2 sensitive peptide) with RGD containing synthetic peptides for cell adhesion and K and Q peptides that can react with tissue transglutaminase (tTG) in the treated tissue that create additional linkages between hydrogel microgel particles over time after delivery.

Within the first 3 formulations, multiple methods of annealing have been developed including (i) photo-activated annealing, (ii) enzymatic annealing, and (iii) synthetic chemical annealing.

Formulation 4-5 have one key differences from formulations 1-3: Replacement of the MMP-2 sensitive peptide with a hyaluronic acid that has been modified to contain free thiols. For this example, the thiolated HA was purchased from ESI Bio (see Methods section for more details).

### Cytocompatibility Testing

Prior to animal experiments, formulations 1-3 and formulations 4-5 were tested (all in the presence and absence of annealing chemistry) for cytocompatibility using immortalized and primary cells. The WST-8 proliferation assay was used to assess cell viability and compare to positive (vehicle) and negative (cytotoxic agent) controls. The formulations used are listed in Table 5 below, along with their characteristics.

TABLE 5
Formulations used in the in vitro cytocompatibility testing

| Formulation | Polymer backbone | Crosslinker | Annealing agent |
|---|---|---|---|
| 1 | PEG-VS 20 kDa | MMP-2 sensitive peptide | Light, 520 nm, Eosin Y |
| | | | (LI) |
| 2 | PEG-VS 20 kDa | MMP-2 sensitive peptide | None (no annealing, NA) |
| 3 | PEG-VS 20 kDa | MMP-2 sensitive peptide | PETMA (P) |
| 4 | PEG-VS 20 kDa | Thiolated HA | None (no annealing, NA) |
| 5 | PEG-VS 20 kDa | Thiolated HA | Light, 520 nm, Eosin Y |

In vitro experiments were performed in well plate format. Cells were grown in culture media in adherent culture, and then exposed to either (i) hydrogel microgel particle suspensions, (ii) hydrogel microgel particle suspensions actively undergoing the annealing process, or (iii) the reaction conditions of the annealing process including soluble components but in the absence of hydrogel microgel particles. After exposure, cells were grown in adherent culture for up to 5 days, measuring proliferation at 1, 3, and 6 days after exposure.

### In Vivo Rat Intradermal Injections

After bench and in vitro testing, the formulations were assessed for basic tissue response in a rat model of intradermal (ID) injection.

TABLE 6
Formulation conditions and comparator used

| | Polymer | | Annealing | Particle size | |
|---|---|---|---|---|---|
| Formulation | Backbone | Crosslinker | Agent | ranges | |
| 2 | PEG-VS 20 kDa | MMP-2 | n/a | 80-140 | μm |
| | | sensitive peptide | | | |
| 3 | PEG-VS 20 kDa | MMP-2 | PETMA | 80-140 | μm |
| | | sensitive peptide | | | |
| 4 | PEG-VS 20 kDa | Thiolated HA | n/a | 160-200 | μm |

| | | | | |
|---|---|---|---|---|
| Juvederm ® XC | As packaged | n/a | n/a | n/a |

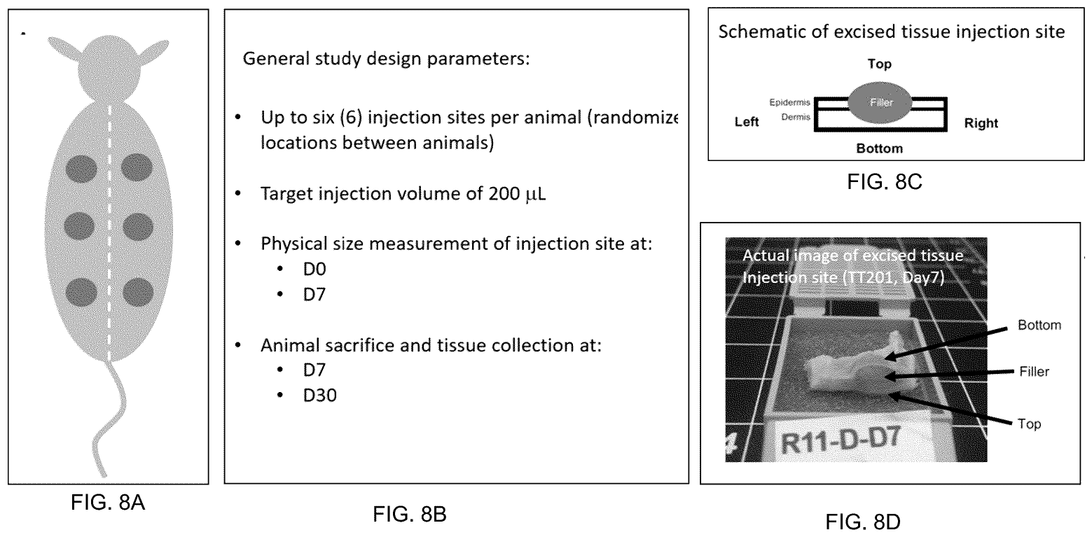

The rat ID model utilized dorsal ID injections on male rats, with three contralateral injection sites on each animal (6 total injection sites). Each injection site received ˜200 μL of material. FIGS. 8A-8D illustrate the anatomical injection schematic used in the in vivo study and timing of measurements. Table 6 outlines the test formulations, controls, and comparators used.

### Biological Endpoints

The in vivo measurements assessed were: (1) injection site swelling after 7 days, (2) histologic evaluation of tissue response in excised injection sites after 7 days and 30 days, and (3) histologic evaluation of material degradation after 7 and 30 days.

### Methods

### Particle Synthesis

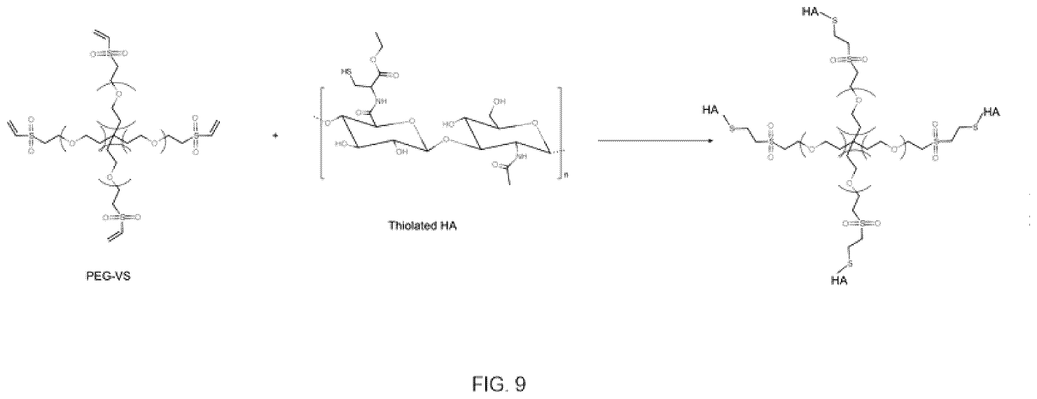

During the hydrogel microgel particle synthesis, the vinyl sulfone (VS) groups of the 4-ARM-PEG-VS react with the thiol groups of hyaluronic acid (HA) or the thiols of the cysteine in the MMP-2 sensitive peptide to covalently link the polymers and cross linker together to form the hydrogel mesh (FIG. 9). This reaction occurs by a thiol-ene Michael addition in presence of a base catalyst (triethanolamine, TEOA). This chemical reaction is very selective with no by-products and quantitative conversion forming a hydrolytically stable thioether bond.

Briefly, a solution of thiolated HA (from ESI-Bio, Catalog #GS220) at 24 mg/mL ([SH]=4.5 mM) in water was mixed 1:1 (v/v) with a 5% (wt) solution of 4-ARM-PEG-VS with 1 mM RGD (in 300 mM TEOA at pH 7.75). The gelation mixture (12 mL) was injected in 490 mL of mineral oil (with 1% wt span80) and stir for 2 h at 1250 rpm at room temperature. The particles were left to settle down overnight before decanting oil. The particles were then purified by TFF using 95% IPA and lyophilized. After lyophilization, the particles were reconstituted in final formulation buffer solution (PBS with 5 μM Eosin Y @pH 7.4) at a volume fraction of 80%. HA-MAP was loaded in 1 mL syringes for dermal injection in rats.

The elastic modulus after light exposure for 10 min at 40 cm was measured using Instron. The size of the particles was determined by laser diffraction particle size analyzer. Endotoxin and bioburden levels were measured by a contract test facility (Ultimate Labs, Inc) per USP<85> and <USP61>, respectively.

### In Vitro Cytocompatibility Testing

3T3 fibroblasts were grown to 90% confluent and then suspended in DMEM at 2.5×10⁶ cells/mL. Then, 2 μL of cell suspension was mixed with MAP (see Table 1 for description of the different formulations tested for cytocompatibility) and added to a 96-well plate. For the photo-annealing condition, formulations 1 and 5 were exposed to light using the Philips Burton Super Exam LED (SELED) light for 10 min at 40 cm for photo-annealing. For the chemical annealing, formulation 3 was mixed with PETMA to a final concentration of 0.1 mM. 100 μL of DMEM was added to each well. Then, the plate was incubated at 37° C. with 5% CO₂ for 1, 3 and 6 days. Cell viability was then measured at 450 nm using WST-8 kit.

### In Vivo Rat Intradermal Injections

Sprague Dawley male rats were purchased from Charles River Laboratories (Crl:SD, outbred strain), weighing between 200 g and 300 g at time of receiving. All in vivo studies were performed as prescribed in the Animal Care and Use Protocol (ACUP) 20C340L1 at Absorption systems California, Inc (ASC).

Briefly, animals were received and acclimated per ASC SOP. On injection day, animals were anesthetized via isoflurane/oxygen gas mixture. Injection sites were clipped and disinfected with isopropanol prior to injection. Injections were performed, targeting “200 μL injection into the intradermal space of each site. Injections were made using a 26 Ga luer lock needle fitted to a 1 cc plastic syringe.

Animals were monitored clinically every day for 5 days post injection. After 7 days, animals again anesthetized using isoflurane/oxygen gas mixture. Images were taken of each injection site and measurements of the width and length of the injection site using calipers were made. Animals were monitored weekly until 30 days after injection, upon which the animals were euthanized using isoflurane overdose followed by cervical dislocation.

After euthanasia, tissue siters were collected, ensuring to keep the subdermal space under the injection site intact. Tissue was immediately placed in 4% buffered formaldehyde (pH 7.4) and fixed at room temperature (−20° C.) for 12 hours, after which the tissue was transferred to Ethanol and shipped for paraffin embedding.

Paraffin embedded tissue was sectioned at 5 μm thickness, mounted, and stained using Hematoxylin and Eosin (H&E). Whole slide scans were taken using a 40× objective. Tissue embedding, cutting, staining, and whole slide scanning were outsourced to a contracting lab (Histowiz, Inc).

### Results

The characteristics of formulation 4 are presented in Table 7. The size of the particles was determined by laser diffraction particle size analyzer. This batch passed the acceptance criteria for bioburden (<1 CFU/mL) and endotoxin (<0.5 EU/mL).

TABLE 7
Characteristics after particle synthesis

| | | |
|---|---|---|
| Elastic Modulus (EM), swollen | 29,503 | Pa |
| Standard Deviation (SD) of EM | 51 | Pa |
| Microgel particle Diameter Mean | 203.2 | μm |
| Microgel particle Diameter Mode | 203.5 | μm |
| Microgel particle Diameter SD | 58.7 | μm |

| | |
|---|---|
| Microgel particle Diameter | 28.9% |
| Coefficient of Variation (CV) | |

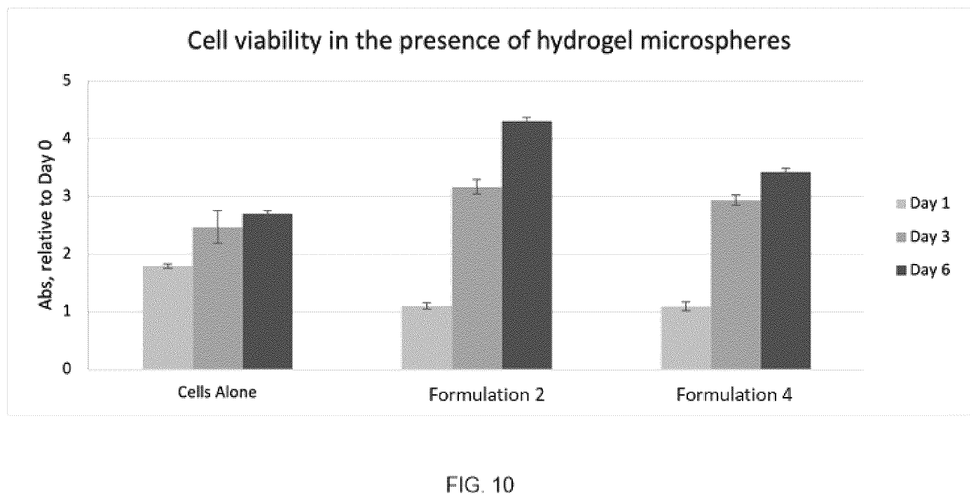

Cytocompatibility testing assessed the effects of various hydrogel microgel particle compositions and annealing reactions on cell viability. Overall, there does not appear to be a measurable effect on cell viability from formulation 4 to formulation 2 (FIG. 10). Formulation 2 has already been assessed for biocompatibility using full ISO-10993 based testing, which included cytocompatibility testing using L929 cells. The equivocal results between formulation 4 and formulation 2 indicate no cytotoxic effects from formulation 4.

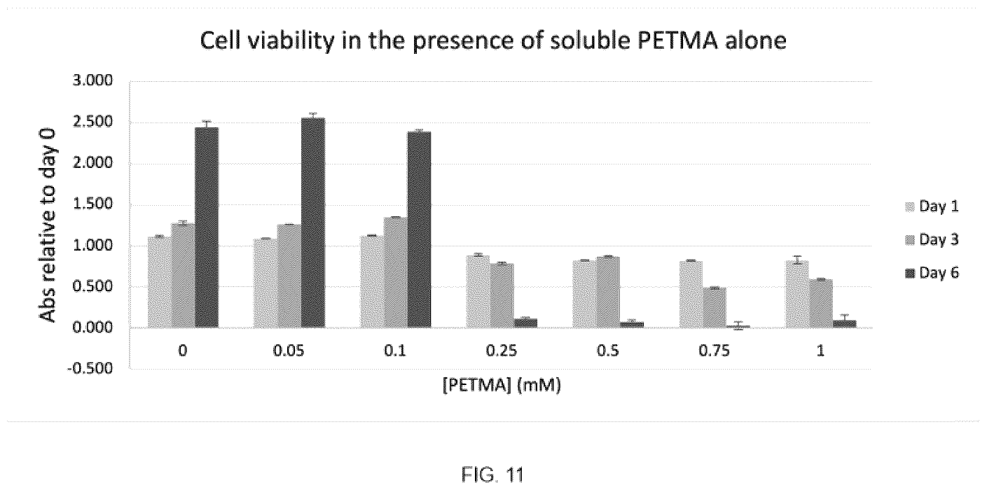

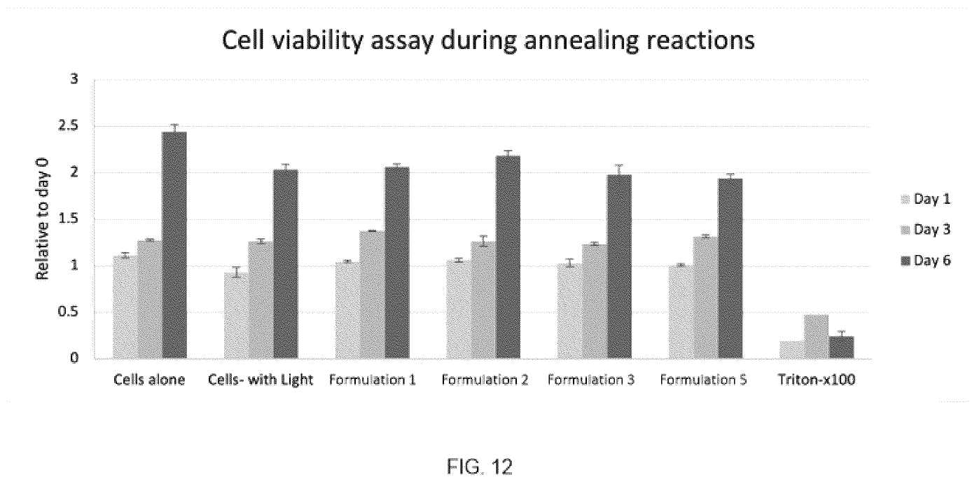

There is a dose dependent cytotoxic effect from PETMA (FIG. 11). Cytotoxic effects are observed at concentrations above 0.1 mM, upon which the effects are significant. However, when concentrations of 0.1 mM PETMA are used in the full annealing reaction (FIG. 12, formulation 3) there is not an observed effect. Formulation 5 also appears to have no observed cytotoxic effect. FIG. 11 illustrates a comprehensive assessment of cell viability in the presence of various annealing reactions. There is no measurable cytotoxicity in this assay when comparing all conditions against cells alone. Triton-x100 served as the positive control for cytotoxicity.

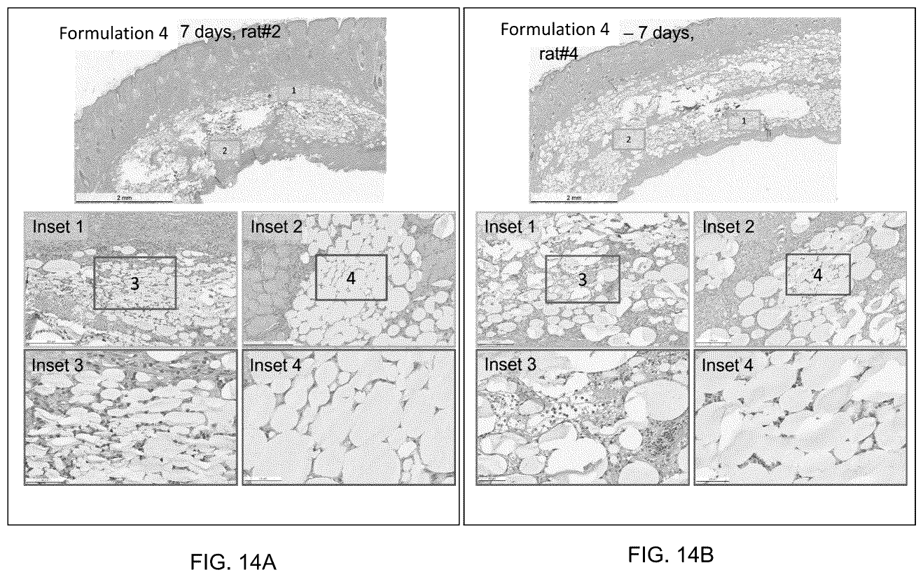

FIGS. 14A-14B illustrate histology of injection sites for formulation 4 after 7 days from two animals. Low magnification images (top two images) illustrate the presence of significant scaffolding material, however due to processing artifacts the centers appear to be removed. This processing artifact occurs when there is weak material or little tissue integration (indicating as such in the center of the injection sites after 7 days). Insets 1 and 2 of both low mag images indicate some cellular infiltration into the pores. Insets 3 and 4 illustrate that there are immune cells present within the scaffold. There does not appear to be a foreign body or multinucleate giant cell (MNGC) response forming at this time point. Scale bars for top two images are 2 mm, Scale bars for insets 1 and 1 are 200 μm. Scale bars for insets 3 and 4 are 100 μm.

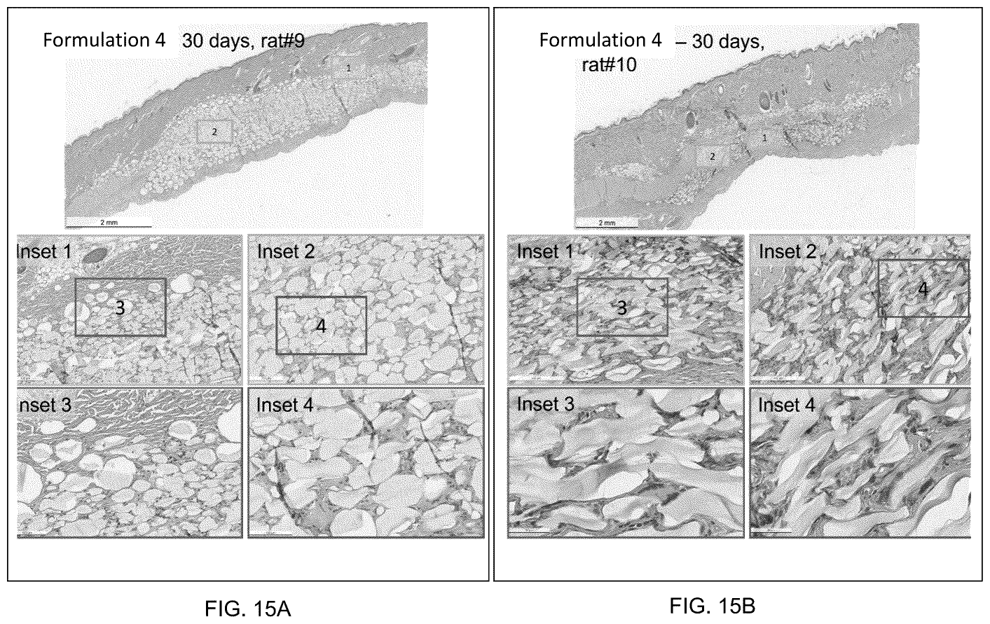

FIGS. 15A-15B illustrate histology of injection sites for formulation 4 after 30 days from two animals. Low magnification images (top two images) illustrate variability in scaffold volume at this time point. This may be due to tissue collection artifacts (e.g., sectioning in the center of a site or the edge). In both cases, where scaffolding is present, there is greater cellular infiltrate and protein deposition compared to day 7 (FIG. 9). Insets 1 and 2 illustrate cellular infiltration, and insets 3 and 4 enable identification of a fibroblast like cellular morphology within the pores. There does not appear to be appreciable MNCG response after 30 days. Scale bars for top two images are 2 mm, Scale bars for insets 1 and 1 are 200 μm. Scale bars for insets 3 and 4 are 100 μm.

FIGS. 16A-16D illustrate comparison of injection sites for all four formulations after 30 days. Formulation 2 displays as expected, with significant degradation. Formulation 3 has significant immune infiltrate, likely due to PETMA reaction. Formulation 4 displays low immune infiltrate and significant new protein deposition within the formed pores, although pores are larger due to lack of any annealing reaction. Juvéderm® does not show any cellular infiltrate to the injection site.

Notable histologic findings for formulation 4 after 30 days. As shown in FIG. 17, although the pore structure is not well defined due to lack of annealing, there is some cellular infiltrate and new vessel formation within the scaffold. There is new protein deposition within the pores, and there appears to be little to no MNGC response at the periphery.

### Example 3: Microgel Particle Synthesis and Effects of Stiffness on Dermal Filler Systems

The objectives of this study were to: (i) develop a strategy for pre-modification of hyaluronic acid to produce thiolated HA to be used as a raw material in the microgel particle synthesis, (ii) investigate how to tune microgel particle stiffness using the modified HA formulation strategy, (iii) select three formulation candidates with varying stiffness profiles for future testing in vivo, and (iv) develop an alternative to PETMA synthetic chemical annealing that has a stronger cytocompatibility profile (e.g., cytocompatible at concentrations at least 5× higher than the working concentrations during annealing.

### Formulations

Here modification of the HA with a cysteine ethyl ester method was used. The following aspects of the pre-modification reaction were investigated: (i) Hyaluronic acid molecular weight (based on vendor certificate of testing), and (ii) degree of substitution (measured using Ellman's assay after modified HA purification).

The ability to make microgel particles with varying stiffnesses was investigated, using the different modified HA produced. These tests used 80 μI hydrogel particles with the same formulation as the microgel particles as surrogates to measure the mechanical properties. These hydrogel particles enable direct measurement using a compressive test on an Instron mechanical analyzer fitted with a 3 mm diameter anvil.

Here, the use of a soluble 3.4 kDa linear PEG that is bifunctional with free thiols on each end was investigated. These free thiols react with the remaining VS on the surface of each microgel particle, linking the microgel particles together where their surfaces are touching (and linking microgel particles to neighboring tissue that contains thiol reactive moieties).

Cytocompatibility was also used to screen the appropriateness of the different annealing agents. In this testing, both the soluble annealing agents themselves alone were tested on cells, as well as directly exposing cells to the active annealing reaction in the presence of the microgel particles. The following formulations were tested in the cytocompatibility testing:

TABLE 8
Formulations tested in the cytocompatibility testing

| | | | Annealed |
|---|---|---|---|
| Formu- | Microgel | Annealing | Compressive |
| lation | particle | Agent | Modulus (Pa) |
| 1 | 4-ARM PEG-VS, | Eosin Y, Light | 1,500-10,000 |
| | 20 kDa | (520 nm) | |
| | MMP-2 sensitive | | |
| | peptide | | |
| 2 | 4-ARM PEG-VS, | Linear PEG-thiol | 1,500-10,000 |
| | 20 kDa | (PEG-SH), | |
| | MMP-2 sensitive | 3.4 kDa | |
| | peptide | | |
| 3 | 4-ARM PEG-VS, | 4-ARM PEG-SH, | 1,500-10,000 |
| | 20 kDa | 20 kDa | |
| | MMP-2 sensitive | | |
| | peptide | | |
| 4 | 4-ARM PEG-VS, | Linear PEG-thiol | 1,500-10,000 |
| | 20 kDa | (PEG-SH), | |
| | Thiolated HA, | 3.4 kDa | |
| | 10 kDa | | |

The biological measurements assessed were: (i) cellular proliferation in vitro at 1, 3, and 6 days post exposure, and (ii) percent survival (%) of cells after 6 days in vitro.

### Methods

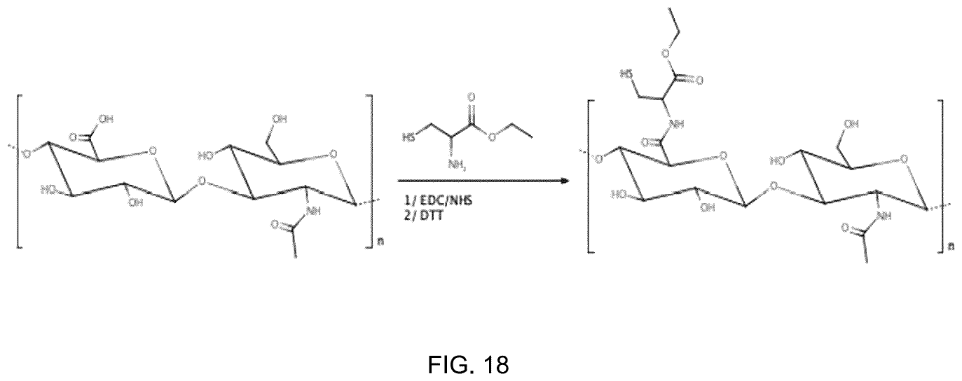

HA of different molecular weights (10-250 kDa, from Stanford Chem and HAWorks) was thiolated using cysteine ethyl ester in the presence of NHS/EDC (FIG. 18). Briefly, 10 mg/mL solution of hyaluronic acid (HA) was adjusted to pH 5 with 1 M HCl. EDC (5 eq) and NHS (5 eq) were added to the solution for activation of the carboxylic group. After 30 minutes of stirring, the pH was adjusted to 6.0 and cysteine ethyl ester (5 eq) was added. The mixture was stirred for 72 h at room temperature under argon. Then, the mixture was purified by dialysis (regenerated cellulose, MWCO: 3,500 Da, Spectra/Por®3) against acidified water (pH 3.5) for 24 h. Then, the pH of the solution was adjusted to 7.5 before adding DTT (2 eq) to reduce any disulfide bonds potentially formed during the reaction. The reduction was performed for 2 h under argon at room temperature. Then, the mixture was purified by dialysis (regenerated cellulose, MWCO: 3,500 Da, Spectra/Por®3) against acidified water (pH 3.5) for 48 h before lyophilization. The thiol content was determined by Ellman's assay.

During the microgel particle synthesis, the vinyl sulfone (VS) groups of the 4-ARM-PEG-VS react with the thiol groups of hyaluronic acid (HA) to covalently crosslink the polymers and HA together to form the hydrogel mesh (FIG. 9). This reaction occurs by a thiol-ene Michael addition in presence of abase catalyst (triethanolamine, TEOA at pH >7.0). This chemical reaction is known to be very selective with no by-products and quantitative conversion forming a hydrolytically stable thioether bond.

Here, the goal was to develop three formulations of different stiffnesses while maintaining the same particle size (80-140 μm) (see Table 9 for targeted stiffness). The stiffness of the microgel particles is tuned by adjusting the ratio of thiol to VS (r=[SH]/[VS]). Increasing the thiol concentration (by increasing the concentration of SH-HA) while maintaining the VS concentration constant leads to increased stiffness (true up to a certain ratio). To identify what concentration of thiolated HA (SH-HA) was required to reach the targeted stiffness from Table 2, a gelation curve (mechanical titration) was performed where different concentrations of SH-HA were mixed with the same polymer solution. After 24 h of reaction, the stiffness (elastic compressive modulus, EM) of the gels before and after swelling (swelling in water for 24 h) was measured using an Instron. A gelation curve must be done for each batch of SH-HA as the gelation depends on the MW and degree of substitution of HA.

TABLE 9
Targeted stiffness of gels before and after swelling

| Formu- | Unswollen | Swollen |
|---|---|---|
| lation | stiffness (Pa) | stiffness (Pa) |
| 4a | 15000-25000 | 1500-10000 |
| 4b | 35000-45000 | 10000-20000 |
| 4c | 75000-85000 | 20000-50000 |

Briefly, a solution of thiolated HA in water at the concentration identified from the gelation curve for the targeted stiffness was mixed 1:1 v/v with a 15% wt solution of 4-ARM-PEG-VS in 300 mM TEOA at pH 8.5. The gelation mixture was injected in 500 mL of mineral oil (with 1% wt span80) and stir for 24 h at 1000-1400 rpm at room temperature. Because the swelling of the particles is highly dependent upon the crosslinking density of the 3D mesh network, the particle size would change for each targeted stiffness if agitation speed were kept constant. To maintain identical particle size between batches, the agitation speed must be adjusted for each run. Then, 500 μL of acetic acid was added to the mixture to stop the reaction, the mixture was stirred for an additional 2 h before stopping agitation. The particles were left to settle down for 24 h before decanting. The particles were then purified by TFF using 95% IPA and lyophilized. After lyophilization, the particles were reconstituted in PBS solution (pH 6-8.5) for 24 h before testing.

A fluorescent dye CF647 was conjugated to the particles either by adding CF647-maleimide directly to the SH-HA solution before synthesis (thiol groups reacts with the maleimide groups) or by adding CF647-hydrazide after synthesis before TFF purification (hydrazide reacts with the free carboxylic groups of HA).

The elastic moduli of the surrogate gels were measured before and after swelling using Instron. The particle size was measured using a Beckman LS Laser Diffraction Particle Size Analyzer. Fluorescent microscopy images were taken for each batch using an ECHO Revolve microscope. The thiol concentration for each SH-HA solution prepared was determined by Ellman's assay.

In the rat intradermal study (see Example 2), MAP was annealed using 0.1 mM PETMA (Pentaerythrivol tetras(2-mercaptoacetate)), a 4-thiol containing crosslinker. The thiol groups react with the free vinyl sulfones on the microgel particles in a thiol-ene Michael addition reaction. However, cytocompatibility assays showed potential cytotoxicity for concentrations ≥0.25 mM. Furthermore, the particles fully anneal within 5 min at pH 7.4, which does not give much time for injection. Even lowering the pH to 6.5 does not give much more time (<10 min) before it is annealed. Therefore, other annealing reagents were investigated including PEG-dithiol (PEG-(SH)₂) 1, 1.5 and 3.4 kDa and 4-ARM-PEG-SH 20 kDa. The optimization of the chemical annealing was performed with formulation 1-3. However, the annealing method identified for formulations 1-3 was also selected for formulations 4-5. Briefly, gels initially formulated at a volume fraction of 100% was thoroughly mixed with the crosslinkers (0.1-0.2 mM) at different pHs, which led to diluting gels to 80%. The elastic modulus was measured at different time points using Instron.

Then, the different HA-MAP formulations were annealed using the selected annealing agent at 0.2 mM at pH 7.4. To investigate the effect of lidocaine on annealing, formulation 4 was annealed using 0.2 mM PEG-dithiol 3.4 kDa at pH 7.4 with the addition of 0.3% wt lidocaine pre-mixed with HA-MAP. Elastic modulus over time was measured using Instron.

The final volume fraction for annealing was 80% for all annealing conditions.

### Cytocompatibility Testing In Vitro

3T3 fibroblasts were grown to 90% confluent and then suspended in DMEM at 2.5×10⁶ cells/mL. Then, 2 μL of cell suspension was mixed with HA-MAP and added to a 96-well plate. For the chemical annealing, HA-MAP was mixed with different crosslinkers (4-ARM-PEG-SH and linear PEG-(SH)₂) to a final concentration of 0.1-0.2 mM at pH 7.4. To test the cytocompatibility of the crosslinkers alone, 4-ARM-PEG-SH (20 kDa) and linear PEG-(SH)₂ (3.4 kDa) were tested at different concentrations. 100 μL of DMEM was added to each well. Then the plate was incubated at 37° C. with 5% CO₂ for 1, 3 and 6 days. Cell viability was then measured at 450 nm using WST-8 kit.

### Bench Testing

### Degradation of Formulation 4 by Hyaluronidase

The degradability of formulation 4 was investigated using hyaluronidase. Briefly, 100 μL of HA-MAP was added to a 48-well plate and annealed with PEG-(SH)₂ (3.4 kDa) for 1 h. Then, 100 μL of hyaluronidase (0.33 mg/mL, 100 Unit/mL) was added to annealed HA-MAP, and microscopy images were taken after 1, 4, 6 and 24 h of incubation at 37° C.

### Break Force and Extrusion Force

The extrusion force of formulation 4 loaded in 1-cc syringe with a 30G needle was measured on an Instron at 10 mm/min. The extrusion force was compared to water (negative control) and Juvéderm® (positive control). The experiment was performed on different batches of formulation 4.

### Results

The thiol content (degree of substitution, DS) in HA was determined by Ellman's assay for each batch. The average DS per molecular weight is presented in Table 10. Similar degrees of substitution were obtained for MW comprised between 10 and 150 kDa. In contrast, the DS significantly decreased for the 250 kDa HA due to diminished solubility with increased MW at the pH the reaction was run (5-7.5).

TABLE 10
Average degree of substitution after thiolation
of HA for each molecular weight (MW)

| Molecular | Degree of |
|---|---|
| Weight, MW (kDa) | Substitution, DS (%) |
| 10 | 12 ± 4 |
| 50 | 14 ± 6 |
| 150 | 11 ± NA* |
| 250 | 5 ± 2 |

*Only one batch was made

An example of gelation curve is provided for 10 kDa and 50 kDa HA in FIGS. 19A-19B. The stiffness of the gels (unswollen and swollen) increases with an increase of HA concentration up to about 60 mg/mL for both MW (10 and 50 kDa). Above this concentration, the stiffness starts decreasing as the number of available thiols becomes greater than the number of available and accessible VS groups. It is interesting to note that while the gelation curve of unswollen gels for the 50 kDa HA is very similar to the one of the 10 kDa HA, the gelation curve of swollen gels is very distinct between both MW. The swollen gels are significantly stiffer for the 50 kDa compared to the 10 kDa indicating less swelling due to an increase in the crosslinking density. Stiffer swollen particles can be obtained with the 50 kDa HA compared to the 10 kDa for the same HA concentration.

The thiol concentration was measured by Ellman's assay at each SH-HA concentration for both MW (10 kDa and 50 kDa) and FIG. 20 shows their linear dependance.

Initially, the three formulations (4a, 4b, and 4c) were all made using 10 kDa SH-HA to avoid discrepancy in swelling ratio from one molecular weight to another. However, different batches of 10 kDa SH-HA were used for the three formulations, therefore a gelation curve was made for each batch (not shown) and a specific concentration of SH-HA was identified for each formulation (Table 11) based on the targeted stiffness for unswollen and swollen gels (Table 9).

TABLE 11
Identified concentration of 10 kDa
SH-HA required for each formulation

| Formu- | HA MW | SH-HA | SH-HA |
|---|---|---|---|
| lation | (kDa) | run# | concentration |
| 4a | 10 | R18 | 35 |
| 4b | 10 | R21 | 36 |
| 4c | 10 | R20 | 40 |

The thiol concentration, mechanical properties and particle size for each HA-MAP formulation are shown in Table 12. The unswollen and swollen stiffnesses were within specification (based on Table 9) indicating that the stiffness can be tuned appropriately by only adjusting the thiolated HA concentration. The mode for particle size was within specification for all formulations (80-140 μm), however the mean size was slightly less than 80 μm for 4b and 4c. This is because, for these two formulations, the fluorescent dye was added post-synthesis unlike 4a for which the dye was added prior to synthesis. The addition of the dye post-synthesis induced the particles to slightly shrink making difficult the particle size prediction and optimization. Therefore, for future synthesis, it was decided to always add the dye prior to synthesis. Eventually, the agitation speed could be increased to compensate for particle shrinkage. Lastly, it was noted that the standard deviation (and by result the CV) was larger for 4c than for the other two formulations. After further investigation, it appears that the size distribution is broader and more difficult to control for stiffer batches. To circumvent this issue, different concentrations of surfactant (Span80) are currently being explored.

TABLE 12
Synthesis information and results of mechanical properties
and particle size for HA-MAP formulations

| | | [PEG- | | | | Unswollen | Swollen |
|---|---|---|---|---|---|---|---|
| | Synth | VS] | [SH] | r | Agitation | EM (PA) ± | EM (Pa) ± |
| Name | Run# | (% wt) | (mM) | [SH]/[VS] | Speed (rpm) | SD | SD |
| 4a | 7 | 15 | 12.5 | 0.41 | 1250 | 16067 ± 200 | 2450 ± 8 |
| 4b | 14 | 15 | 12.9 | 0.43 | 1250 | 47774 ± 690 | 2450 ± 454 |
| 4c | 10 | 15 | 14.1 | 0.47 | 1300 | 83441 ± 6113 | 22400 ± 779 |

| Name | Mean (μm) | Mode (μm) | SD (μm) | CV (μm) |
|---|---|---|---|---|
| 4a | 95 | 106 | 23 | 24 |
| 4b | 73 | 80 | 20 | 27 |
| 4c | 75 | 116 | 44 | 59 |

First, the effect of different thiol-containing crosslinkers (annealing agents) on the annealing kinetic of formulations 1-3 was investigated at pH 7.4 (FIG. 21). Whereas gels crosslinked with PETMA annealed within 5 min, the PEG-based crosslinkers gave significantly more time before annealing starts (>10 ma) and before completion (>30 m). No significant differences between the different PEG-based crosslinkers were observed. Because 4-ARM-PEG-SH 20 kDa showed significant cytotoxicity (see below) and because lower MW of PEG (1 kDa and 1.5 kDa) are more difficult to procure than 3.4 kDa, PEG-dithiol 3.4 kDa was selected as the annealing agent. For each crosslinker, different pHs and different concentrations were tested. FIG. 22 shows an example of the effect of pH on the annealing kinetic. Decreasing the pH from 7.4 to 6.5 significantly slows down the kinetic (the annealing reaction does not start before 30 min).

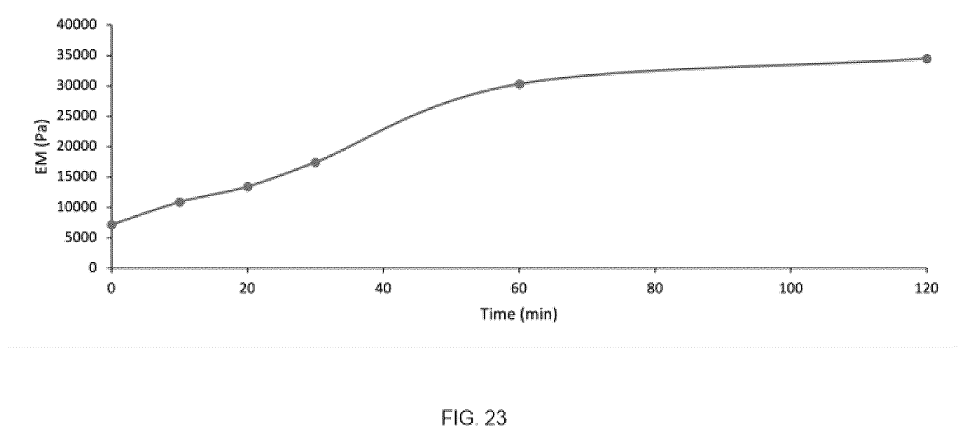

Table 13 summarizes the data of chemical annealing using 0.2 mM of PEG-dithiol 3.4 kDa after 2 h. The elastic modulus after annealing increases with stiffness as expected. The kinetic for 4c was also evaluated to confirm it follows the same trend as formulations 1-3 (FIG. 23). 4c is fully annealed within 60 min.

TABLE 13
Elastic modulus before and after 2 h of annealing
using 0.2 mM of PEG-dithiol 3.4 kDa at pH 7.4.

| | EM before | EM after 2 h |
|---|---|---|
| Formu- | annealing | annealing |
| lation | (Pa) ± SD | (Pa) ± [symbol] |
| 4a | 1685 ± 262 | 10312 ± 1914 |
| 4b | 1890 ± 392 | 29822 ± 10738 |
| 4c | 7137 ± 498 | 34461 ± 8184 |

[symbol]

indicates data missing or illegible when filed

FIGS. 24A-24C show the effect of lidocaine on the kinetic of annealing for three different batches of 4a. Overall, lidocaine (0.3% wt) reduces the final stiffness after annealing and slightly slows down the reaction.

### Cytocompatibility Testing In Vitro

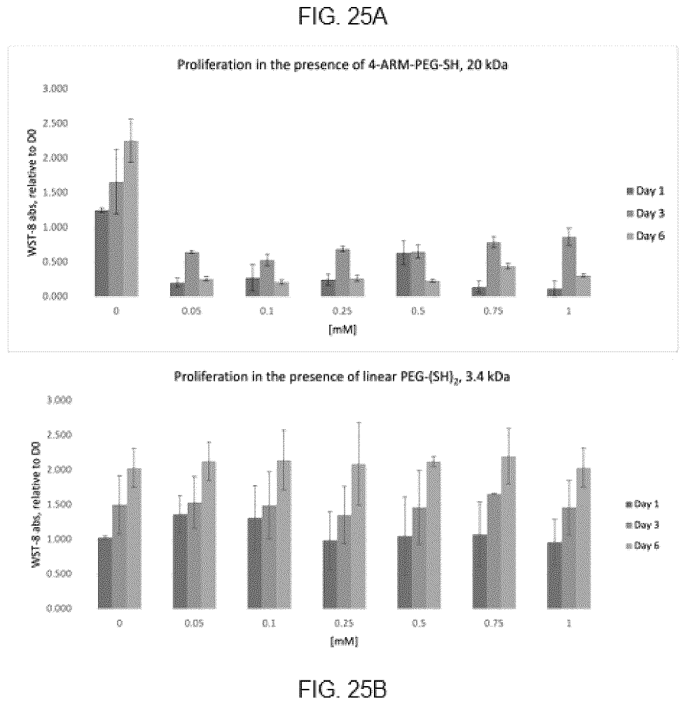

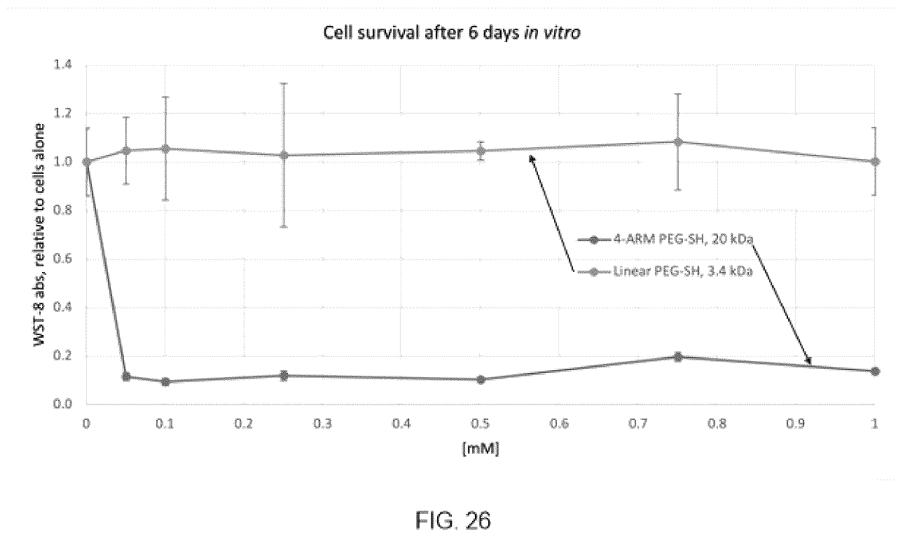

Initial studies evaluated the cytocompatibility of the thiolated 4-arm PEG (4-ARM-PEG-SH, 20 kDa) and a thiolated linear PEG (linear PEG-(SH)₂, 3.4 kDa). Surprisingly, there was a stark difference in the cytocompatibility profiles between the two (FIGS. 25A-25B). There is no detectable effect of the linear PEG over all the concentrations tested, while the 4-ARM-PEG-SH displays marked cytotoxicity at all concentrations tested. FIG. 26 illustrates the cell survival fraction after 6 days in vitro for both annealing agents. There is no effect on survival for the linear PEG-(SH)₂ over all concentrations tested.

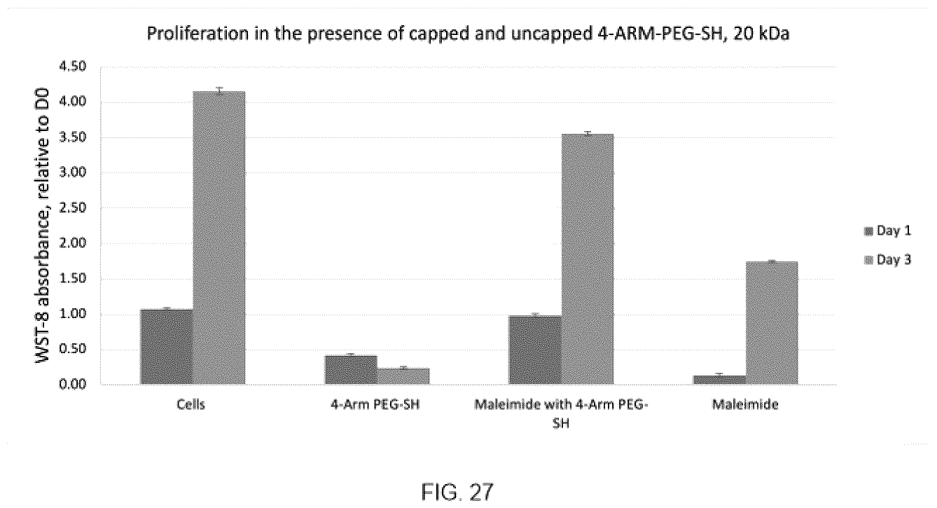

Further, capping of the free thiols on the 4-ARM-PEG-SH restores the cytocompatibility (removing the cytotoxic effect) (FIG. 27). Note that capping was performed by pre-reacting the 4-ARM-PEG-SH with free maleimide. This solution was then applied directly to the cells without purification. This indicates that the observed cytotoxic affects seen in FIGS. 11 and 12 were likely due to the molecule itself, and not due to a contaminant in the solution or the raw material.

Based on these data, it appeared that the linear PEG-(SH)₂ was a better candidate for an annealing agent compared to the 4-ARM-PEG-SH. None-the-less, further testing of both annealing agents was performed to determine the cytocompatibility of each reaction in the presence of the entire system (annealing agent and microgel particles).

To assess the cytocompatibility of the full annealing reactions, cells were directly exposed to the full synthetic chemical annealing process in the presence of microgel particles for both the 4-ARM-PEG-SH and linear PEG-(SH)₂ annealing agents at 0.1 mM and 0.2 mM, respectively. Please refer to Table 8 for detailed description of each formulation.

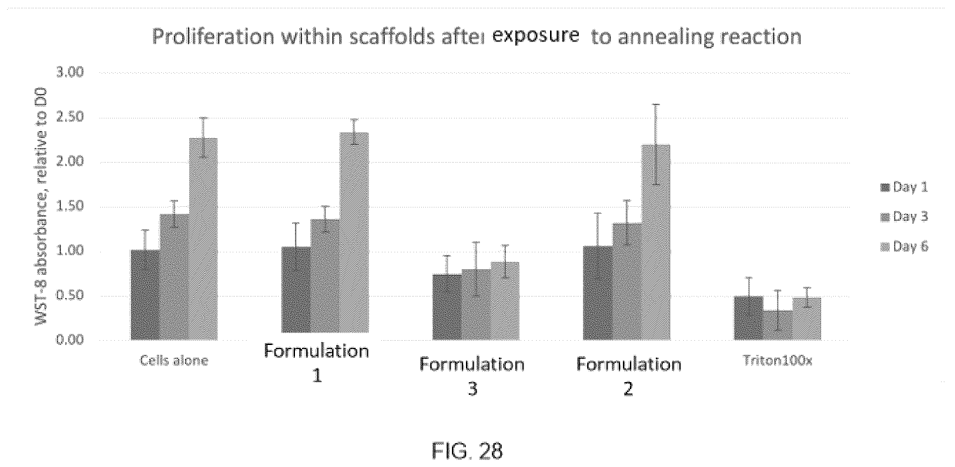

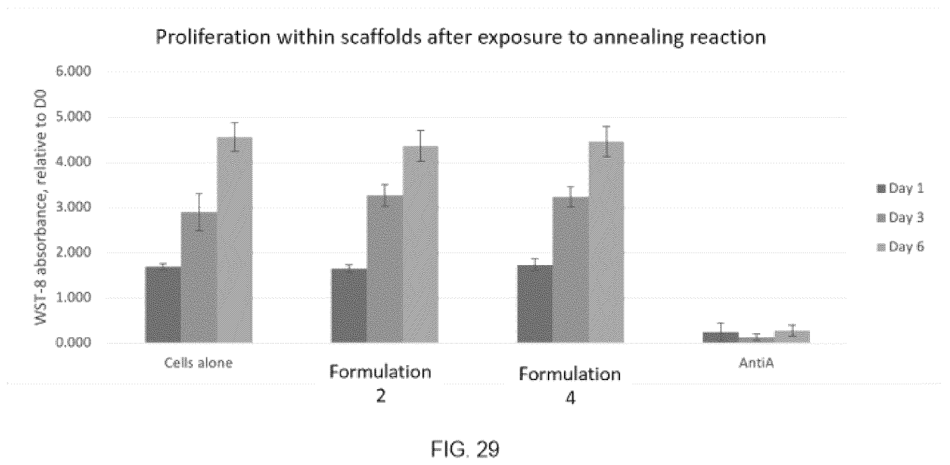

The full system annealing data were generated in two separate experiments performed on different days, with some conditions repeated between the two experiments. FIG. 28 illustrates the cell proliferation data from the first test, FIG. 29 shows the data from the second test. Note that the difference in magnitude of the WST-8 signals between experiments is expected, as the experiments were performed on separate days. Each test was internally controlled with positive controls and negative controls (Triton-x100 for experiment 1 and Anti-A for experiment 2).

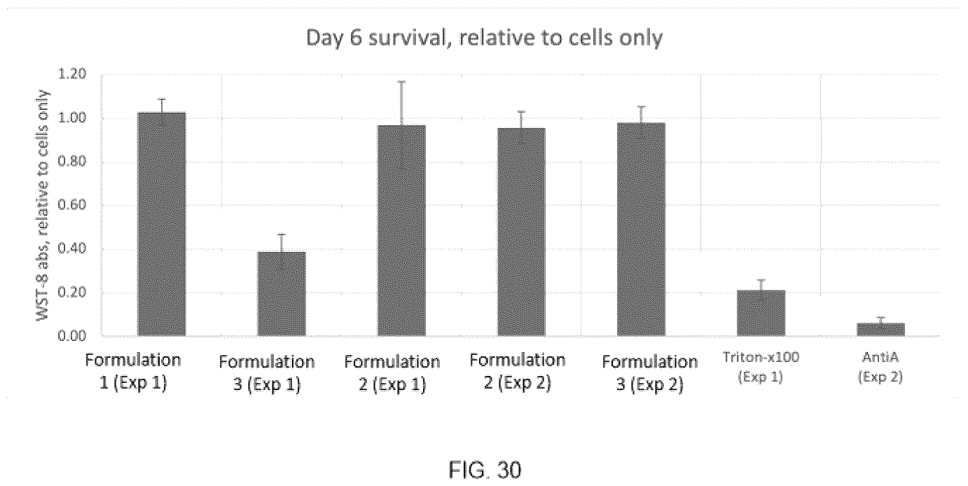

In order to directly compare all the conditions, the day 6 WST-8 absorbance values for each condition were normalized to the day 6 cells alone (the negative control). This enabled presentation of the data in a normalized format, relative to the cells alone condition (FIG. 30).

Degradation of HA hydrogel particles using 100 Unit/mL hyaluronidase was monitored by microscopy overtime (FIG. 31). After 4 h, the degradation of the particles was significant, and after 6 h no residual particles were observed. It is important to note that the concentration of hyaluronidase used in this experiment was about 20 times lower than the one commonly used in clinic.

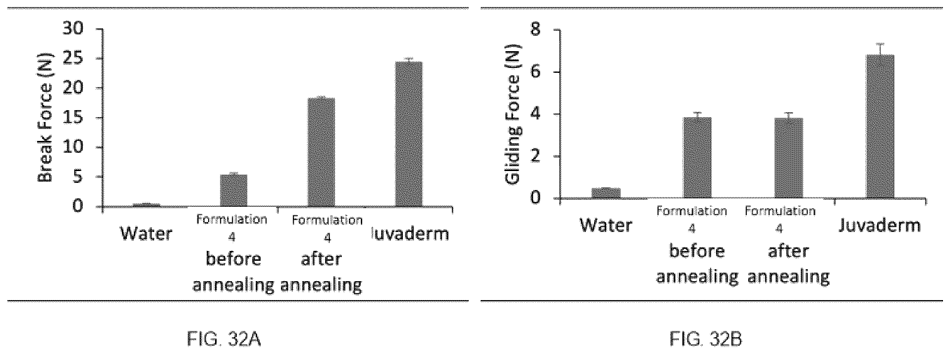

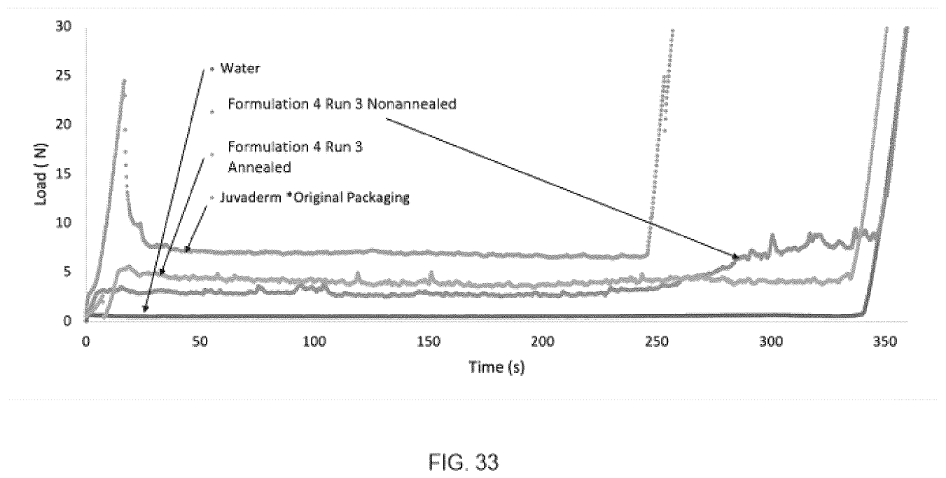

Formulation 4 was significantly easier to extrude from the syringe than Juvéderm XC®. Annealed formulation 4 requires a higher break force than non-annealed, and gliding force was similar between the two (FIGS. 32A-32B and FIG. 33).

### Example 4: Terminal Sterilization

Various product formats will be investigated in this example. The formats used will include but are not limited to:
(i.) Hydrated, Ready to use (RTU) as a two-component system (annealing agent may be lyophilized or RTU as well)
(ii.) Lyophilized format, either as single component or two-component system (e.g., annealing agent is lyophilized with the microgel particles or lyophilized in a second container)
(iii.) Hybrid lyophilized and RTU format as a two-component system, in which microgel particles are lyophilized and annealing agent is RTU
(iv.) Processed via terminal sterilization or aseptically manufactured (tbc)

These product formats will be exposed to increasing doses of terminal sterilization methods including, but not limited to, those outlined in Table 14.

TABLE 14
methods and doses (or conditions) of
terminal sterilization techniques

| | Irradiative Method | | | |
|---|---|---|---|---|
| | Min dose (kGy) | | Max Dose (kGy) | |
| | Gamma or ebeam | | | |
| | 1 | | 50 | |
| Temperature | Min Temp. | Min Time | Max Temp. | Max Time |
| Method | (° C.) | (min) | (° C.) | (min) |
| Moist heat | 100 | 5 | 121 | 45 |
| Dry heat | 60 | 5 | 150 | 45 |

To measure the effects of the terminal sterilization conditions, the product candidates will be tested according to the testing outlined in Table 15. Each candidate will be tested against these criteria prior to entering the terminal sterilization condition and after exiting the terminal sterilization condition. For those product formats involving lyophilization, the products will be reconstituted as necessary for testing (both before and after the sterilization condition is applied).

TABLE 15
Test name

pH of final product prior to injection
Osmolality (mOsmol/kg)
Microgel particle size (μm) measured by laser diffraction
Volume fraction before mixing with crosslinker
Volume fraction after mixing with crosslinker
Elastic modulus of microgel particles
Elastic modulus of annealed scaffold
Free Vinyl Sulfone (VS) content before annealing
Free Vinyl Sulfone (VS) content after annealing
Apparent Viscosity (Pa*s) shear rate of 0.1, 1.0, & 10 s⁻¹
Storage Modulus, G′ (Pa)
Loss Modulus, G″ (Pa)
Extrusion Force through a specified sized needle (10 mm/min)
Impurities by RP-HPLC
Annealing rate
(Time to reach 90% completion of reaction)
Lidocaine content (mg/mL)
Extractable fraction of HA (GelC)

The results of this testing will identify an acceptable sterilization dose/condition that the product candidates can withstand. Based on the identified maximum dose/condition, an assessment will be made to calculate the maximum bioburden value in the product PRIOR to terminal sterilization at that condition in order to achieve a predicted SAL of 10⁻⁶. This calculated maximum bioburden will then inform the level of control needed over upstream processing during the manufacturing process.

If there are no terminal sterilization conditions evaluated that enable the potential to achieve an SAL of 10⁻⁶, then an assessment of the manufacturing cost of goods (COGs) when using aseptic manufacturing will be performed.

### Example 5: Rat Model Using Dermal Filler Systems Described Herein

A rat model of subcutaneous (SC) injection will be used to evaluate the dermal filler systems described herein. The purpose of the model is to evaluate the tissue response and inflammation associated with each formulation from relative to selected comparators for the target indications of jawline, midface, and cheek. Generally, injections will be performed on healthy animals, with clinical observations over time ending in animal sacrifice and collection of tissue sites for histological analysis. Multiple injections will be performed on each animal, and each animal will be exposed to test and comparator articles.

Table 16 describes the overview of the study. The biological endpoints and method of assessments are listed in Table 17. Rat skin will be tattooed 1 week prior to injections using a circle to mark the injection site (and enable more accurate tissue collection at longer timepoints). Any inflammation from tattooing will be allowed to subside during the 1-week period from tattooing and prior to performing injections

TABLE 16
Study design overview of Rat model

| | | | | Statistical | Terminal |
|---|---|---|---|---|---|
| | Starting | Type of | Number of | sample | Time |
| | weight | injection and | injections | sizing | points |
| Species | range (g) | location | per animal | (N)[1] | (days) |
| Rodent | 200-250 | Subcutaneous | 6 | 6 | 2, 7, 30, |
| (rat) | | (SC), dorsum | | | 90, 180 |
| Rodent | 200-250 | Subcutaneous | 6 | 3 | 2, 7, 30, |
| (rat) | | (SC), dorsum | | | 90, 180 |

[1]Statistical sample sizing based on number of injections

TABLE 17
Biological endpoints and methods of assessment:

| Biological endpoint | Method of assessment |
|---|---|
| In vivo swelling ratio | Clinical observation - |
| at injection site | Use High frequency ultrasound (HF-US)[2] |
| | for volumetric analysis if available. |
| Cellular infiltration | Histology (IHC Ki67 corroborated with H&E) |
| into and proliferation | nuclei per high powered field at specified |
| within the injection | distances from injection site/tissue |
| site scaffold over time | interface |
| | percent of cells that are Ki67-postive per |
| | high powered field at specified distances |
| | from injection site/tissue interface |
| Collagen deposition | Histology (Picrosirius Red and Herovici |
| within the injection | corroborated by H&E) |
| site scaffold over | Density of Picrosirius Red staining per |
| time | high powered field at specified distances |
| | from injection site/tissue interface |
| | Polarized light imaging of picrosirius red |
| | stains to evaluate relative amounts of |
| | collagen I and collagen III per high |
| | powered field at specified distances from |
| | injection site/tissue interface |
| | Collagen neosynthesis assessed by |
| | Herovici staining |
| Elastin content within | Histology (Verhoeff's von Gieson, VVG, |
| the injection site | Movat or IHC (tbc), corroborated by H&E) |
| scaffold over time | Density of VVG, Movat or IHC (tbc) stain |
| | per high powered field at specified |
| | distances from injection site/tissue |
| | interface |
| Inflammation assessed | Total immune infiltrate assessed via H&E |
| by histology | by a board certified veterinary or dermato- |
| | pathologist to semi quantitate: |
| | Polymorphonuclear cells |
| | Lymphocytes |
| | Macrophages |
| | Multinucleate giant cells (MNGCs) |
| | Presence of histiocytosis/granuloma |
| | within and around injection site scaffold. |
| Degradation of the | Histology (H&E) |
| Test and control | Quantitative analysis of the degradation (as |
| articles in the | detected by histology) over time. Analysis |
| injection sites | performed by a veterinary pathologist |
| over time | % Degradation will be calculated relative |
| | to the earliest histologic time point taken |
| | (this earliest timepoint will be defined as |
| | “100% remaining”). |
| | Image analysis of H&E-stained slides will |
| | be performed, calculating the cross- |
| | sectional area of the injection site. |
| | Sections for degradation analysis will be |
| | taken as close to the center of the injection |
| | site as possible. |
| | Option to use High frequency ultrasound (HF- |
| | US)[3] for volumetric analysis if available. |
| Bulk RNA | The 6 implant sites of the 3 additional rats per |
| sequencing | time point will be stored in ‘RNAlater’ for |
| | further RNAseq if needed after Go/No Go |
| | decision based on the POC data |

[2],[3]Visual Sonics Vevo 2100 micro-ultrasound small animal imaging system or equivalent

The formulations will be compared to Restylane® Lyft (a hyaluronic acid filler) and Sculptra® (a poly-L-lactic acid facial infection), with phosphate buffered saline (PBS) as a control. Based on the information in Table 16 and the test, comparator, and control conditions listed, it is estimated that at least six (6) animals per timepoint will be required and totaling a minimum of 30 animals for all testing except RNA testing. An additional 15 animals will be included, and samples collected and frozen in ‘RNAlater’ for RNA testing once POC is confirmed.

The biological endpoints will be assessed throughout the course of the study in accordance with the schedule of events outlined in Table 18.

TABLE 18
Schedule of events during the Rat study

| | | | | | | |
|---|---|---|---|---|---|---|
| | After | | | | | |
| Biological | injection | 2 | 7 | 30 | 90 | 180 |
| Endpoint | (Day 0) | days | days | days | days | days |

| | | | | | |
|---|---|---|---|---|---|
| Cellular | X | X | X | X | X |
| infiltration/ | | | | | |
| vascularization | | | | | |
| Inflammation, | X | X | X | X | X |
| Foreign | | | | | |
| Body Response, | | | | | |
| Histiocytosis/ | | | | | |
| Granuloma | | | | | |
| Collagen | ◯ | ◯ | X | X | X |
| Elastin | ◯ | ◯ | X | X | X |
| Degradation | X | X | X | X | X |
| RNA | X | X | X | X | X |

‘X’ means this data will be used for endpoint analysis

‘O’ means the data may be taken as ‘for information only, (FIO)’, because the tissue will already be collected for another endpoint; however, the timepoint may be too early to see a response. Cellular infiltration may be observed within 7 days, beginning primarily with innate immune cells (macrophages, neutrophils) and followed by other dermal cells such as fibroblasts. The cellular infiltration may persist, and cellular proliferation may be seen as early as 30 days and persist.

Vascularization may be observed by 30 days, as evidenced by patient blood vessels (vessels with red blood cells within the lumen) present inside the pores of the scaffold. Vascularization and maturation may continue over the next 60 days (to 90 days total).

Initial acute inflammation may be observed at 7 days and reduced by 30 days. Even further reduction in inflammatory cell infiltrate may continue over the course of the study. After 30 days, the primary cell type present may be macrophages that are secondary to scaffold degradation.

Some MNGCs may be present at 7 and 30 days. There may be less MNGCs than in the biostimulator (Sculptra) conditions. Any MNGS observed in the dermal filler systems described here are expected to resolve after 30-60 days and be no longer visible histologically after 90 days.

Collagen may be present within the pores after 30 days. Collagen may continue to persist throughout the study timeframe.

Elastin may be present in the pores (or in the presence where the scaffold has degraded) after longer time points, potentially after 90 days.

The dermal fillers described herein may degrade slowly over the course of the study. The dermal fillers may be completely degraded in this model by 90 days.

RNA profiling of the injection sites for the dermal fillers described here may show less chronic inflammation compared to the biostimulator conditions, and show more extracellular matrix production compared to the dermal filler comparator (Restylane).

### Example 6: Minipig Model Using Dermal Filler Systems Described Herein

A minipig model of subcutaneous (SC) injection will be used to compare degradation kinetics and inflammation relative to the rat study performed in Example 5. Test and comparator articles identical to the rat study will be used in this minipig study. Multiple injections will be performed on each animal, and each animal will be exposed to test and comparator articles. Table 19 describes the overview of the study. The biological endpoints and method of assessments are listed in Table 20. Minipig skin will be tattooed 1 week prior to injections using a circle to mark the injection site (and enable more accurate tissue collection at longer timepoints). Any inflammation from tattooing will be allowed to subside during the 1-week period from tattooing and prior to performing injections.

TABLE 19
Study design overview of Minipig model

| | | | | Statis- | |
|---|---|---|---|---|---|
| | Starting | | | tical | Terminal |
| | weight | Type of | Number of | sample | Time |
| | range | injection and | injections | sizing | points |
| Species | (kg) | location | per animal | (N) [4] | (days) |
| Gottingen | 20-30 | Subcutaneous | up to 20 | 3 | 7, 30, 90, |
| Minipig | | (SC), dorsum | (if dorsum) | | 180 |
| (swine) | | or post- | up to 6 | | |
| | | auricular | (if post- | | |
| | | | auricular) | | |

[4] Statistical sample sizing based on number of injections

Based on the information in Table 19 and the test, comparator, and control conditions listed, it is estimated that at least three (3) animals per timepoint will be required and totaling a minimum of 12 animals.

TABLE 20
Biological endpoints and methods of assessment

| Biological endpoint | Method of assessment |
|---|---|
| In vivo swelling ratio | Clinical observation - |
| at injection site | Use High frequency ultrasound (HF-US) [5] |
| | for volumetric analysis if available. |
| Cellular infiltration | Histology (IHC Ki67 corroborated with H&E) |
| into and proliferation | nuclei per high powered field at |
| within the injection | specified distances from injection |
| site scaffold over time | site/tissue interface |
| | percent of cells that are Ki67-postive per |
| | high powered field at specified distances |
| | from injection site/tissue interface |
| Collagen deposition | Histology (Picrosirius Red and Herovici |
| within the injection | corroborated by H&E) |
| site scaffold over | Density of Picrosirius Red staining per |
| time | high powered field at specified distances |
| | from injection site/tissue interface |
| | Polarized light imaging of picrosirius |
| | red stains to evaluate relative amounts |
| | of collagen I and collagen III per high |
| | powered field at specified distances |
| | from injection site/tissue interface |
| | Collagen neosynthesis assessed by |
| | Herovici staining |
| Elastin content within | Histology (Verhoeff's von Gieson, VVG, |
| the injection site | Movat or IHC (tbc), corroborated by H&E) |
| scaffold over time | Density of VVG, Movat or IHC (tbc) |
| | stain per high powered field at specified |
| | distances from injection site/tissue |
| | interface |
| Inflammation assessed | Total immune infiltrate assessed via |
| by histology | H&E by a board certified veterinary or |
| | dermato-pathologist to semi quantitate: |
| | Polymorphonuclear cells |
| | Lymphocytes |
| | Macrophages |
| | Multinucleate giant cells (MNGCs) |
| | Presence of histiocytosis/granuloma |
| | within and around injection site |
| | scaffold. |
| Degradation of the | Histology (H&E) |
| Test and control | Quantitative analysis of the degradation |
| articles in the | (as detected by histology) over time. |
| injection sites | Analysis performed by a veterinary |
| over time | pathologist |
| | % Degradation will be calculated |
| | relative to the earliest histologic time |
| | point taken (this earliest timepoint will |
| | be defined as “100% remaining”). |
| | Image analysis of H&E-stained slides |
| | will be performed, calculating the cross- |
| | sectional area of the injection site. |
| | Sections for degradation analysis will be |
| | taken as close to the center of the |
| | injection site as possible. |
| | Option to use High frequency ultrasound |
| | (HF-US (Visual Sonics Vevo 2100 micro- |
| | ultrasound small animal imaging system or |
| | equivalent)) for volumetric analysis if |
| | available. |

[5] Visual Sonics Vevo 2100 micro-ultrasound small animal imaging system or equivalent

These biological endpoints will be assessed throughout the course of the study in accordance with the schedule of events outlined below in table 21.

TABLE 21
Schedule of events during the Minipig study

| | | | | | |
|---|---|---|---|---|---|
| | After | | | | |
| Biological | injection | | | | |
| Endpoint | (Day 0) | 7 days | 30 days | 90 days | 180 days |

| | | | | |
|---|---|---|---|---|
| Cellular infiltration | X | X | X | X |
| Vascularization | X | X | X | X |
| Collagen | ◯ | X | X | X |
| Elastin | ◯ | X | X | X |
| Inflammation | X | X | X | X |
| Degradation | X | X | X | X |

‘X’ means this data will be used for endpoint analysis
‘◯’ means the data may be taken as ‘for information only, (FIO)’, because the tissue will already be collected for another endpoint; however, the timepoint may be too early to see a response.

Cellular infiltration may be observed within 7 days, beginning primarily with innate immune cells (macrophages, neutrophils) and followed by other dermal cells such as fibroblasts. The cellular infiltration may persist, and cellular proliferation may be seen as early as 30 days and persist.

Vascularization may be observed by 30 days, as evidenced by patient blood vessels (vessels with red blood cells within the lumen) present inside the pores of the scaffold. Vascularization and maturation may continue over the next 60 days (to 90 days total).

Initial acute inflammation may be observed at 7 days and reduced by 30 days. Even further reduction in inflammatory cell infiltrate may continue over the course of the study. After 30 days, the primary cell type present may be macrophages that are secondary to scaffold degradation.

Some MNGCs may be present at 7 and 30 days. There may be less MNGCs than in the biostimulator (Sculptra) conditions. Any MNGS observed in the dermal filler systems described here are expected to resolve after 30-60 days and be no longer visible histologically after 90 days.

Collagen may be present within the pores after 30 days. Collagen may continue to persist throughout the study timeframe.

Elastin may be present in the pores (or in the presence where the scaffold has degraded) after longer time points, potentially after 90 days.

The dermal fillers described herein may degrade slowly over the course of the study. The dermal fillers may be completely degraded in this model by 90 days. The dermal fillers may be completely degraded by 30 days. The dermal fillers may be partially degraded by 30 days.

RNA profiling of the injection sites for the dermal fillers described here may show less chronic inflammation compared to the biostimulator conditions, and show more extracellular matrix production compared to the dermal filler comparator (Restylane).

### Example 7: Pharmacokinetic (PK) Study Using Dermal Filler Systems Described Herein

A pharmacokinetic (PK) model in rats will be used to track the components of the formulations in systemic tissues. The route of administration and dosing will be similar to the rat study described above in Example 5. However, this PK study will include shorter timepoints. Table 22 below describes the overview of the proposed study. The potential biological endpoints and method of assessments are listed in Table 23. Rat skin will be tattooed 1 week prior to injections using a circle to mark the injection site and enable more accurate tissue collection at longer timepoints. Any inflammation from tattooing will be allowed to subside during the 1-week period from tattooing and prior to performing injections.

TABLE 22
Study design overview of PK model

| | | | | | Time |
|---|---|---|---|---|---|
| | | | | | points for |
| | | | | Statistical | PET |
| | Starting | Type of | Number of | sample | imaging or |
| | weight | injection and | injections | sizing | LCS or |
| Species | range (g) | location | per animal | (N) [6] | MS (days) |
| Rodent | 200-250 | Subcutaneous | 6 | 3 | 0.5 h, 2 h, |
| (rat) | | (SC), dorsum | | | 6 h, 12 h, |
| | | | | | and days: |
| | | | | | 1, 2, 3, 5, |
| | | | | | 7, 30, 90, |
| | | | | | 180 |

[6] Statistical sizing is based on number of animals, not number of injection sites.

PBS will be used as a control. Based on the information in Table 22 and the test and control conditions listed, it is estimated that at least three (3) animals per formulation, per timepoint will be required and totaling a minimum of 144 animals. Each formulation may be radiolabeled (each component: PEG, HA, labelled separately, but not the annealing agent). Distributions may be radiolabeled (each component: PEG, HA, labelled separately, but not the annealing agent). Target organs will be harvested at sacrifice.

TABLE 23
Biological endpoints and methods of assessment:

| Biological endpoint | Method of assessment |
|---|---|
| Product distribution in local | For radiolabeled compounds: |
| draining lymph nodes (LN), | PET or Liquid Scintillation Counting |
| spleen, liver, and kidneys - | (LCS) imaging or organ solubilization |
| non exhaustive list of organs | followed by radioactivity counting |
| to be collected | (will depend on the obtained specific |
| | activity of each radiolabeled compound) |
| | For the annealing agent: |
| | Mass Spectrometry |
| Product distribution in blood/ | Blood/Plasma levels of device components |
| plasma and effects on blood | Blood panel and CBC |
| panel | |

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1-235. (canceled)

236. A dermal filler system, comprising:
a) microgel particles comprising a hydrogel polymer and a thiol or a derivative thereof, wherein the hydrogel polymer comprises hyaluronic acid (HA), poly(ethylene glycol) (PEG), polylactic acid (PLA), or a combination thereof; and
b) vinyl sulfone (VS) or a derivative thereof, wherein the microgel particles undergo an annealing reaction to form a porous covalently stabilized scaffold, wherein either of (i) the thiol or the derivative thereof or (ii) the vinyl sulfone or the derivative thereof is present in the dermal filler system in excess of the other.

237. The dermal filler system of claim 236, wherein the hydrogel is a copolymer of the HA and the PEG having approximately identical molecular weights of each of the HA and the PEG.

238. The dermal filler system of claim 236, wherein the hydrogel polymer comprises the thiol or the derivative thereof or the VS or the derivative thereof, or the combination thereof.

239. The dermal filler system of claim 236, wherein the PEG comprises PEG-dithiol.

240. The dermal filler system of claim 236, wherein the HA is modified to comprise the thiol or the derivative thereof to form thiolated-HA.

241. The dermal filler system of claim 236, wherein the thiol or the derivative thereof and the VS or derivative thereof are present in the dermal filler system at a molar ratio of about 1:1.

242. The dermal filler system of claim 236, wherein the thiol or the derivative thereof and the VS or the derivative thereof are in excess of each other in the dermal filler system such that the excess of the thiol or the derivative thereof or the VS or the derivative thereof participates in the annealing reaction to form the porous covalently stabilized scaffold.

243. The dermal filler system of claim 236, wherein the PEG-dithiol is configured to interact with the excess VS or derivative thereof in the annealing reaction to form the porous covalently stabilized scaffold.

244. The dermal filler system of claim 236, wherein the dermal filler system further comprises glutaraldehyde or a derivative thereof, divinyl sulfone or a derivative thereof, 1,4-butanediol diglycidyl ether (BDDE) or a derivative thereof, or any combination thereof configured to interact in a crosslinking reaction to synthesize the microgel particles.

245. The dermal filler system of claim 236, wherein the dermal filler system further comprises two or more acrylates, methacrylates, acrylamides, maleimides, norbornenes, or any combination thereof.

246. The dermal filler system of claim 236, wherein the porous covalently stabilized scaffold is present in the tissue site for at least 18 months before complete degradation.

247. A dermal filler system, comprising:
a) a dermal filler formulation comprising microgel particles, wherein the microgel particles comprise a hydrogel polymer and a thiol or a derivative thereof, wherein the hydrogel polymer comprises hyaluronic acid (HA), poly(ethylene glycol) (PEG), polylactic acid (PLA), or a combination thereof; and
b) vinyl sulfone (VS) or a derivative thereof, wherein the microgel particles undergo an annealing reaction to form a porous covalently stabilized scaffold comprising an elastic compressive modulus of about 1,000 Pascals (Pa) to about 100,000 Pa.

248. The dermal filler system of claim 247, wherein the thiol or the derivative thereof and the vinyl sulfone or the derivative thereof are present in the dermal filler system at a molar ratio of about 0.3 to about 0.8 to achieve the elastic compressive modulus.

249. The dermal filler system of claim 247, wherein the microgel particles are present in a suspension comprising the microgel particles and water and wherein a 50% to 100% volume fraction of the suspension comprises the microgel particles to achieve the elastic compressive modulus.

250. The dermal filler system of claim 247, wherein the hydrogel polymer comprises a degree of substitution per monomer of about 5% to about 20%.

251. The dermal filler system of claim 247, wherein the porous covalently stabilized scaffold comprises an osmolality of about 100 milliosmole per kilogram (mOsmol/kg) to about 400 mOsmol/kg.

252. The dermal filler system of claim 247, wherein the covalently stabilized scaffold comprises an apparent viscosity of 1000 to about 1000000 mPa*s.

253. A method of delivering a dermal filler formulation to a tissue site of a subject, the method comprising: delivering to the tissue site the dermal filler formulation comprising a hydrogel that anneals in vivo to form a porous covalently stabilized scaffold under conditions sufficient to form a cell matrix within the porous covalently stabilized scaffold, wherein the cell matrix forms new tissue at the tissue site while minimizing a foreign body response in the subject.

254. The method of claim 253, wherein the delivering comprises exerting an extrusion force of up to 40 Newtons (N) on the dermal filler formulation.

255. The method of claim 253, wherein the porous covalently stabilized scaffold remains at the tissue site in an amount sufficient to fill at least part of the tissue site for an amount of time that is greater than or equal to 9 months following the delivery
